(19) United States
(12) Patent Application Publication
(10) Pub. No.: US 2025/0305013 A1
(43) Pub. Date: Oct. 2, 2025

(54) Methods and Compositions for Making Amide Compounds

(71) Applicant: Genomatica, Inc., San Diego, CA (US)

(72) Inventors: Harish Nagarajan, San Diego, CA (US); Tae Hoon Yang, San Diego, CA (US); Ali Khodayari, San Diego, CA (US); Shawn Bachan, San Diego, CA (US); Sankha Ghatak, San Diego, CA (US); Nicholas Eakley, San Diego, CA (US); Amit Shah, San Diego, CA (US); Jinel Shah, San Diego, CA (US); Bo Zhang, San Diego, CA (US)

(73) Assignee: Genomatica, Inc., San Diego, CA (US)

(21) Appl. No.: 18/260,225

(22) PCT Filed: Jan. 15, 2022
(86) PCT No.: PCT/US2022/012644

§ 371 Date: Jun. 30, 2023

Related U.S. Application Data
(60) Provisional application No. 63/138,495, filed on Jan. 17, 2021.

(51) Int. Cl. C12P 13/02; C12N 9/06; C12N 15/52

(52) U.S. Cl. CPC C12P 13/02; C12N 9/0016; C12N 15/52; C12Y 104/01004

(57) ABSTRACT

Disclosed are biosynthetic methods and engineered microorganisms that enhance or improve the biosynthesis of 6-aminocaproate, hexamethylenediamine, caproic acid, caprolactone, or caprolactam. The engineered microorganisms are modified to include, for example, upredulated and/or exogenous transporters for 6-aminocaproate, deletions and/or downregulated importers for 6-aminocaproate, upregulated and/or exogenous glutamate dehydrogenase, and/or deletions and/or downregulation of rcsA and/or cpsBG. Other engineered microorganisms may have disruptions of endogenous transporters for 6-aminocaproate.

This application is a 371 of PCT/EP2022/012644, having an international filing date of Jan. 15, 2022, which claims the benefit of U.S. Provisional Application Ser. No. 63/138,495, filed Jan. 17, 2021, the content of which is incorporated by reference in its entirety.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “GMTA048_ST25.txt”, a creation date of Dec. 22, 2023 and a size of 212 kilobytes. The Sequence Listing filed via EFS-Web is part of the specification and is incorporated in its entirety by reference herein.

### BACKGROUND

Nylons are polyamides that can be synthesized by the condensation polymerization of a diamine with a dicarboxylic acid or the condensation polymerization of lactams. Nylon 6,6 is produced by reaction of hexamethylenediamine (HMD) and adipic acid, while nylon 6 is produced by a ring opening polymerization of caprolactam. Therefore, adipic acid, hexamethylenediamine, and caprolactam are important intermediates in nylon production.

Microorganisms have been engineered to produce some of the nylon intermediates. However, engineered microorganisms can produce undesirable byproducts as a result of undesired enzymatic activity on pathway intermediates and final products. Such byproducts and impurities therefore increase, cost, and complexity of biosynthesizing compounds and can decrease efficiency or yield of the desired products.

### SUMMARY

Provided herein are non-naturally occurring microbial organisms having a 6-aminocaproic acid pathway, caprolactam pathway, hexamethylenediamine pathway, caprolactone pathway, 1,6-hexanediol pathway, or a combination of one or more of these pathways. The non-naturally occurring microbial organisms can comprise at least one exogenous nucleic acid encoding an exogenous transporter that exports 6-aminocaproic acid, caprolactam, hexamethylenediamine, caprolactone, and/or hexanediol. The non-naturally occurring microbial organism can comprise a disruption of an endogenous transporter that imports into the cell 6-aminocaproic acid, caprolactam, hexamethylenediamine, caprolactone, and/or hexanediol. The non-naturally occurring microbial organism can comprise at least one exogenous nucleic acid encoding an exogenous glutamate dehydrogenase (e.g., gdhA or homologs thereof, EC Number 1.4.1.4). The non-naturally occurring microbial organism can comprise a disruption of an endogenous gene whose product is involved in the mucoid phenotype. The non-naturally occurring microorganism can include a disruption in a that reduces the production of intermediates and/or products that compete for carbon with the production of 6-aminocaproic acid, caprolactam, hexamethylenediamine, caprolactone, and/or 1,6-hexanediol. The introduction of one or more of these changes to a non-naturally occurring microbial cell with a pathway for making 6-aminocaproic acid, caprolactam, hexamethylenediamine, caprolactone, and/or 1,6-hexanediol, increases the production of 6-aminocaproic acid, caprolactam, hexamethylenediamine, caprolactone, and/or 1,6-hexanediol. The non-naturally occurring microorganism can comprise one or more of the engineered changes described above or below.

The exogenous transporter that exports 6-aminocaproic acid can be, for example, the transporters in Table 16. The non-naturally occurring microbial organisms can comprise an exogenous nucleic acid(s) encoding one or more of SEQ ID NO: 1, 3, 17, 19, 21, 23, 25, 27, 29, 31, 57, 59, 61, 63, 65, 67, 69, 71, 73, 75, 77, 79, 81, 83, 85, 87, 89, 91, and/or 93. The endogenous transporter that imports into the cell 6-aminocaproic acid can be, for example, gabP or homologs thereof, and/or csiR or homologs thereof. The non-naturally occurring microbial organisms can comprise disruptions of the endogenous gabP or homologs thereof, csiR or homologs thereof, or both. The exogenous glutamate dehydrogenase can be, for example, any glutamate dehydrogenase (e.g., Table 17 such as gdhA or homologs thereof, EC Number 1.4.1.4) making glutamate from α-ketoglutarate and whose glutamate product can be used in a transmination reaction in the pathway for making 6-aminocaproic acid, caprolactam, and/or hexamethylenediamine. The non-naturally occurring microbial organisms can comprise an exogenous nucleic acid(s) encoding one or more glutamate dehydrogenases (e.g., Table 17 such as gdhA or homologs thereof). The endogenous gene whose product is involved in the mucoid phenotype can include, for example, rcsA or homologs thereof, or cpsB or homologs thereof (EC Number 2.7.7.13 or 2.7.7.22), or cpsG or homologs thereof (EC Number 5.4.2.8), or cpsBG rcsA or homologs thereof, or cpsB or homologs thereof, or cpsG or homologs thereof, or cpsBG. The non-naturally occurring microbial organisms can comprise a disruption in rcsA or homologs thereof, or cpsB or homologs thereof, or cpsG or homologs thereof, or cpsBG rcsA or homologs thereof, or cpsB or homologs thereof, or cpsG or homologs thereof, or cpsBG, or any combination of the foregoing.

The disruptions which reduce production of carbon competing intermediate and/or products can include, for example, disruptions in the pathways for making adipic acid (e.g., disruptions in sad or homologs thereof, gabD or homologs thereof, and/or ybfF or homologs thereof), 6-hydroxycaproic acid (e.g., disruptions in yghD or homologs thereof, yjgB or homologs thereof, and/or yahK or homologs thereof), and/or gamma-aminobutyric acid (e.g., disruption in gabT or homologs thereof).

A non-naturally occurring microbial organisms can comprise a pathway for making 6-aminocaproic acid, an exogenous nucleic acid(s) encoding ybjE or homologs thereof, and/or yhiM or homologs thereof, and/or a glutamate dehydrogenase (e.g., gdhA or homologs thereof), disruptions of gabP or homologs thereof, and/or csiR or homologs thereof, and/or rcsA or homologs thereof, and/or cpsB or homologs thereof, and/or cpsG or homologs thereof, and/or cpsBG. A non-naturally occurring microbial organism can comprise a pathway for making 6-aminocaproic acid, disruptions of gabP or homologs thereof and rcsA or homologs thereof, and exogenous nucleic acids encoding ybjE or homologs thereof and glutamate dehydrogenase (e.g., gdhA or homologs thereof). A non-naturally occurring microbial organism can comprise a pathway for making 6-aminocaproic acid and can also include disruptions in the pathways for making adipic acid, 6-hydroxycaproic acid, and/or gama-aminobutyric acid.

The non-naturally occurring microbial organisms can comprise exogenous nucleic acids encoding enzymes necessary for producing 6-aminocaproic acid, 1,6-hexanediol, caprolactone, caprolactam, hexamethylenediamine in a sufficient amount to produce the respective product. In some cases, one or more of these exogenous nucleic acids may be heterologous to the non-naturally occurring microbial organisms.

The non-naturally occurring microbial organisms can have a pathway for making a C6 product (e.g., 6-aminocaproic acid, caprolactam, hexamethylenediamine, caprolactone, 1,6-hexanediol, and/or adipic acid). The non-naturally occurring microbial organisms can comprise an exogenous nucleic acid encoding an exogenous transporter that exports the C6 product. The non-naturally occurring microbial organisms can comprise one or more disruptions of endogenous transporters that import the C6 product. The non-naturally occurring microbial organisms can comprise disruptions of endogenous genes for rcsA and/or cpsBG. The non-naturally occurring microbial organism can comprise disruptions in pathways that make intermediates and products that compete for carbon with the desired C6 product.

The non-naturally occurring microbial organisms can have a pathway for making a C5-C14 product. The non-naturally occurring microbial organisms can comprise an exogenous nucleic acid encoding an exogenous transporter that exports the C5-C14 product. The non-naturally occurring microbial organisms can comprise one or more disruptions of endogenous transporters that import the C5-C14 product. The non-naturally occurring microbial organisms can comprise disruptions of endogenous rcsA and/or cpsBG. The non-naturally occurring microbial organism can comprise disruptions in pathways that make intermediates and products that compete for carbon with the desired C5-C14 product.

Also disclosed are methods for producing 6-aminocaproic acid, caprolactam, and/or hexamethylenediamine. The methods can include culturing a 6-aminocaproic acid, caprolactam, and/or hexamethylenediamine producing non-naturally occurring microbial organisms, where the non-naturally occurring microbial organisms express exogenous nucleic acid(s) encoding ybjE or homologs thereof, and/or yhiM or homologs thereof, and/or a glutamate dehydrogenase (e.g., gdhA or homologs thereof), and/or the non-naturally occurring microbial organism has disruptions of gabP or homologs thereof, and/or csiR or homologs thereof, and/or rcsA or homologs thereof, and/or cpsB or homologs thereof, and/or cpsG or homologs thereof, and/or cpsBG or homologs thereof, and/or the non-naturally occurring microbial organism has disruptions for carbon competing intermediates and/or products including, for example, disruptions in the pathways for making adipic acid (e.g., disruptions in sad or homologs thereof, gabD or homologs thereof, and/or ybfF or homologs thereof), 6-hydroxycaproic acid (e.g., disruptions in yghD or homologs thereof, yjgB or homologs thereof, and/or yahK or homologs thereof), and/or gamma-aminobutyric acid (e.g., disruption in gabT or homologs thereof). The methods include culturing the non-naturally occurring microbial organisms under conditions and for a sufficient period of time to produce 6-aminocaproic acid, caprolactam, hexamethylenediamine.

Methods of producing 6-aminocaproic acid (6ACA) can comprise culturing an appropriate non-naturally occurring microbial organism described above for a sufficient time period and under suitable conditions for producing 6ACA. The methods can further include recovering 6ACA from the microbial organism, fermentation broth, or both.

Methods of producing hexamethylene diamine comprise culturing an appropriate non-naturally occurring microbial organism described above for a sufficient time period and under suitable conditions for producing hexamethylene diamine. The methods can further include recovering hexamethylene diamine from the microbial organism, fermentation broth, or both. The non-naturally occurring microbial organism can comprise two, three, four, five, six, seven or more exogenous nucleic acid sequences each encoding a hexamethylene diamine pathway enzyme.

Also disclosed are methods for producing a C6 product (e.g., 6-aminocaproic acid, caprolactam, hexamethylenediamine, caprolactone, 1,6-hexanediol, and/or adipic acid). The methods can include culturing a C6 producing non-naturally occurring microbial organisms, where the microbial organism express exogenous nucleic acid(s) encoding a transporter that exports the C6 product, and the microbial organism has disruptions of rcsA or homologs thereof, and/or cpsB or homologs thereof, and/or cpsG or homologs thereof, and/or cpsBG or homologs thereof, and/or endogenous transporters that import the C6 product to the cell, and/or steps for making intermediates and/or products that compete for carbon with the desired C6 product. The methods include culturing the non-naturally occurring microbial organisms under conditions and for a sufficient period of time to produce the C6 product.

Methods of producing 6-aminocaproic acid, 1,6-hexanediol, caprolactone, caprolactam, hexamethylenediamine comprising culturing an appropriate non-naturally occurring microbial organism disclosed above for a sufficient time period and conditions for producing 6-aminocaproic acid, 1,6-hexanediol, caprolactone, caprolactam, hexamethylenediamine. The methods can further include recovering 6-aminocaproic acid, 1,6-hexanediol, caprolactone, caprolactam, and/or hexamethylenediamine from the microbial organism, fermentation broth, or both. The non-naturally occurring microbial organism can comprise two, three, four, five, six or seven exogenous nucleic acid sequences each encoding 6-aminocaproic acid, 1,6-hexanediol, caprolactone, caprolactam, hexamethylenediamine pathway enzymes.

Disclosed herein are methods for producing a desired C5-C14 product. The methods can include culturing a C5-C14 producing non-naturally occurring microbial organisms, where the microbial organisms express exogenous nucleic acid(s) encoding a transporter that exports the desired C5-C14 product, and the microbial organism has disruptions of rcsA, and/or cpsBG, and/or endogenous transporters that import the desired C5-C14 product to the cell, and/or steps for making intermediates and/or products that compete for carbon with the desired C5-C14 product. The methods include culturing the non-naturally occurring microbial organisms under conditions and for a sufficient period of time to produce the desired C5-C14 product.

The 6-aminocaproic acid pathway can comprise: (i) transaminase, (ii) 6-aminocaproate dehydrogenase, or both (iii) transaminase and 6-aminocaproate dehydrogenase enzymes. The non-naturally occurring microbial organism can further comprise one or more additional exogenous nucleic acids encoding one or more of the 6-aminocaproic acid pathway enzymes. The exogenous nucleic acids encoding one or more of the 6-aminocaproic acid pathway enzymes can be heterologous to the microbial organism.

The non-naturally occurring microbial organism can comprise a hexamethylenediamine pathway. The hexamethylenediamine pathway can comprise (i) 6-aminoacaproyl CoA transferase, (ii) 6-amino caproyl CoA synthase, (iii) 6-amino caproyl CoA reductase, (iv) hexamethylenediamine transaminase, (v) hexamethylenediamine dehydrogenase, (v) or a combination of one or more of the enzymes (i)-(v). The microbial organism can further comprise one or more additional exogenous nucleic acids encoding one or more of the hexamethylenediamine pathway enzymes. The exogenous nucleic acids encoding one or more of the hexamethylenediamine pathway enzymes can be heterologous to the microbial organism.

The non-naturally occurring microbial organism can comprise a caprolactam pathway. The caprolactam pathway can include an aminohydrolase enzyme. The microbial organism can further comprise one or more additional exogenous nucleic acids encoding an aminohydrolase enzyme. The exogenous nucleic acids encoding aminohydrolase enzyme can be heterologous to the microbial organism.

The non-naturally occurring microbial organism can comprise a 1, 6-hexanediol pathway. The 1, 6-hexanediol pathway can comprise one or more of the following enzymes: a 6-aminocaproyl-CoA transferase or synthetase catalyzing conversion of 6ACA to 6-aminocaproyl-CoA; a 6-aminocaproyl-CoA reductase catalyzing conversion of 6-aminocaproyl-CoA to 6-aminocaproate semialdehyde; a 6-aminocaproate semialdehyde reductase catalyzing conversion of 6-aminocaproate semialdehyde to 6-aminohexanol; a 6-aminocaproate reductase catalyzing conversion of 6ACA to 6-aminocaproate semialdehyde; an adipyl-CoA reductase adipyl-CoA to adipate semialdehyde; an adipate semialdehyde reductase catalyzing conversion of adipate semialdehyde to 6-hydroxyhexanoate; a 6-hydroxyhexanoyl-CoA transferase or synthetase catalyzing conversion of 6-hydroxyhexanoate to 6-hydroxyhexanoyl-CoA; a 6-hydroxyhexanoyl-CoA reductase catalyzing conversion of 6-hydroxyhexanoyl-CoA to 6-hydroxyhexanal; a 6-hydroxyhexanal reductase catalyzing conversion of 6-hydroxyhexanal to HDO; a 6-aminohexanol aminotransferase or oxidoreductases catalyzing conversion of 6-aminohexanol to 6-hydroxyhexanal; a 6-hydroxyhexanoate reductase catalyzing conversion of 6-hydroxyhexanoate to 6-hydroxyhexanal; an adipate reductase catalyzing conversion of ADA to adipate semialdehyde; and an adipyl-CoA transferase, hydrolase or synthase catalyzing conversion of adipyl-CoA to ADA.

The non-naturally occurring microbial organism can comprise pathways from adipate or adipyl-CoA to caprolactone. These pathways from adipate or adipyl-CoA to caprolactone can comprise one or more of the following enzymes: adipyl-CoA reductase, adipate semialdehyde reductase, 6-hydroxyhexanoyl-CoA transferase or synthetase, 6-hydroxyhexanoyl-CoA cyclase or spontaneous cyclization, adipate reductase, adipyl-CoA transferase, synthetase or hydrolase, 6-hydroxyhexanoate cyclase, 6-hydroxyhexanoate kinase, 6-hydroxyhexanoyl phosphate cyclase or spontaneous cyclization, phosphotrans-6-hydroxyhexanoylase.

The non-naturally occurring microbial organism can comprise a species of Acinetobacter, Actinobacillus, Anaerobiospirillum, Aspergillus, Bacillus, Clostridium, Corynebacterium, Escherichia, Gluconobacter, Klebsiella, Kluyveromyces, Lactococcus, Lactobacillus, Mannheimia, Pichia, Pseudomonas, Rhizobium, Rhizopus, Saccharomyces, Schizosaccharomyces, Streptomyces, and Zymomonas. The non-naturally occurring microbial organism can be a strain of Escherichia. coli.

In an aspect, the disclosure relates to a non-naturally occurring microbial organism comprising a pathway for making a 6-aminocaproic acid and an exogenous nucleic acid encoding a transporter for the 6-aminocaproic acid, wherein the exogenous transporter exports the 6-aminocaproic acid from the cell.

The non-naturally occurring microbial organism described above, wherein the non-naturally occurring organism produces a 6-aminocaproic acid and wherein the production of the 6-aminocaproic acid by the microbial organism is increased compared to a microbial organism without the exogenous nucleic acid.

The non-naturally occurring microbial organisms described above, wherein the exogenous transporter is a YbjE or a homolog thereof.

The non-naturally occurring microbial organisms described above, wherein the exogenous transporter is a YhiM or a homolog thereof.

The non-naturally occurring microbial organisms described above, further comprising a second nucleic acid encoding a second exogenous transporter, wherein the second exogenous transporter is a YhiM or a homolog thereof.

The non-naturally occurring microbial organisms described above, wherein the at least one exogenous nucleic acid is chromosomally integrated.

The non-naturally occurring microbial organisms described above, wherein the at least one exogenous nucleic acid is episomal.

The non-naturally occurring microbial organisms described above, wherein the at least one exogenous nucleic acid overexpresses the transporter for the 6-aminocaproic acid.

The non-naturally occurring microbial organism described above, further comprising a disruption of an endogenous nucleic acid encoding a transporter that imports 6-aminocaproic acid into the microbial organism.

The non-naturally occurring microbial organisms described above, wherein the transporter with the disruption is a gabP or a homolog thereof.

The non-naturally occurring microbial organisms described above, wherein the transporter with the disruption is a csiR or a homolog thereof.

The non-naturally occurring microbial organisms described above, wherein the transporter with the disruption is a csiR or a homolog thereof.

The non-naturally occurring microbial organism described above, further comprising a third exogenous nucleic acid encoding a glutamate dehydrogenase.

The non-naturally occurring microbial organisms described above, wherein the glutamate dehydrogenase is a GdhA or a homolog thereof.

The non-naturally occurring microbial organisms described above, wherein the third exogenous nucleic acid is chromosomally integrated.

The non-naturally occurring microbial organisms described above, wherein the third exogenous nucleic acid is episomal.

The non-naturally occurring microbial organisms described above, wherein the third exogenous nucleic acid overexpresses the glutamate dehydrogenase.

The non-naturally occurring microbial organisms described above, further comprising a disruption of a rcsA, a cpsB, a cpsG, or a cpsBG.

The non-naturally occurring microbial organisms described above, wherein the disruption is a disruption of a rcsA or a homolog thereof.

The non-naturally occurring microbial organisms described above, wherein the disruption is a disruption of a cpsB or a homolog thereof.

The non-naturally occurring microbial organisms described above, wherein the disruption is a disruption of a cpsG or a homolog thereof.

The non-naturally occurring microbial organisms described above, wherein the disruption is a disruption of a cpsBG.

In an aspect, the disclosure relates to a non-naturally occurring microbial organisms comprising a pathway for making a 6-aminocaproic acid and a gene with a disruption, wherein the disrupted gene encodes an endogenous transporter for the 6-aminocaproic acid, wherein the endogenous transporter imports the 6-aminocaproic acid into the cell.

The non-naturally occurring microbial organism described above, wherein the non-naturally occurring microbial organism produces 6-aminocaproic acid, and wherein the production of the 6-aminocaproic acid by the microbial organism is increased compared to a microbial organism without the disruption of the gene encoding the endogenous transporter.

The non-naturally occurring microbial organisms described above, wherein the gene with the disruption is a gabP or a homolog thereof.

The non-naturally occurring microbial organisms described above, wherein the gene with the disruption is a csiR or a homolog thereof.

The non-naturally occurring microbial organisms described above, further comprising a second gene with a second disruption, wherein the second gene is a csiR or a homolog thereof.

The non-naturally occurring microbial organisms described above, further comprising an exogenous nucleic acid encoding a glutamate dehydrogenase.

The non-naturally occurring microbial organisms described above, wherein the glutamate dehydrogenase is a GdhA or a homolog thereof.

The non-naturally occurring microbial organisms described above, wherein the exogenous nucleic acid is chromosomally integrated.

The non-naturally occurring microbial organisms described above, wherein the exogenous nucleic acid is episomal.

The non-naturally occurring microbial organisms described above, wherein the exogenous nucleic acid overexpresses the glutamate dehydrogenase.

The non-naturally occurring microbial organisms described above, further comprising a disruption of a rcsA, a cpsB, a cpsG, or a cpsBG.

The non-naturally occurring microbial organisms described above, wherein the disruption is a disruption of a rcsA or a homolog thereof.

The non-naturally occurring microbial organisms described above, wherein the disruption is a disruption of a cpsB or a homolog thereof.

The non-naturally occurring microbial organisms described above, wherein the disruption is a disruption of a cpsG or a homolog thereof.

The non-naturally occurring microbial organisms described above, wherein the disruption is a disruption of a cpsBG.

In an aspect, the disclosure relates to a non-naturally occurring microbial organism comprising a pathway for making a 6-aminocaproic acid and an exogenous nucleic acid encoding a glutamate dehydrogenase, wherein at least some of the glutamate made by the glutamate dehydrogenase is used by a transaminase that produces the 6-aminocaproic acid.

The non-naturally occurring microbial organism described above, wherein the glutamate dehydrogenase is a GdhA or a homolog thereof.

The non-naturally occurring microbial organisms described above, wherein the exogenous nucleic acid is chromosomally integrated.

The non-naturally occurring microbial organisms described above, wherein the exogenous nucleic acid is episomal.

The non-naturally occurring microbial organisms described above, wherein the exogenous nucleic acid overexpresses the glutamate dehydrogenase.

The non-naturally occurring microbial organisms described above, further comprising a second exogenous nucleic acid encoding a transporter for the 6-aminocaproic acid, wherein the transporter exports the 6-aminocaproic acid from the cell.

The non-naturally occurring microbial organisms described above, wherein the exogenous transporter is a YbjE or a homolog thereof.

The non-naturally occurring microbial organisms described above, wherein the exogenous transporter is a YhiM or a homolog thereof.

The non-naturally occurring microbial organisms described above, further comprising a third exogenous nucleic acid, wherein the third exogenous nucleic acid encodes a YhiM, or a homolog thereof.

The non-naturally occurring microbial organisms described above, wherein the second exogenous nucleic acid is chromosomally integrated.

The non-naturally occurring microbial organisms described above, wherein the second exogenous nucleic acid is episomal.

The non-naturally occurring microbial organisms described above, wherein the second exogenous nucleic acid overexpresses the transporter.

The non-naturally occurring microbial organisms described above, further comprising a gene with a disruption, wherein the gene encodes an endogenous transporter for the 6-aminocaproic acid, wherein the endogenous transporter imports the 6-aminocaproic acid into the cell.

The non-naturally occurring microbial organisms described above, wherein the endogenous transporter with the disruption is a gabP or a homolog thereof.

The non-naturally occurring microbial organisms described above, wherein the endogenous transporter with the disruption is a csiR or a homolog thereof.

The non-naturally occurring microbial organisms described above, wherein the endogenous transporter with the disruption is a csiR or a homolog thereof.

The non-naturally occurring microbial organisms described above, further comprising a disruption of a rcsA, a cpsB, a cpsG, or a cpsBG.

The non-naturally occurring microbial organisms described above, wherein the disruption is a disruption of a rcsA or a homolog thereof.

The non-naturally occurring microbial organisms described above, wherein the disruption is a disruption of a cpsB or a homolog thereof.

The non-naturally occurring microbial organisms described above, wherein the disruption is a disruption of a cpsG or a homolog thereof.

The non-naturally occurring microbial organisms described above, wherein the disruption is a disruption of a cpsBG.

In an aspect, the disclosure relates to a non-naturally occurring microbial organism comprising a pathway for making a 6-aminocaproic acid and a disruption of a rcsA, a cpsB, a cpsG, or a cpsBG, wherein the disruption of the rcsA, the cpsB, the cpsG, or the cpsBG reduces the mucoid phenotype of the microbial organism.

The non-naturally occurring microbial organism described above, wherein th3 disruption increases the production of 6-aminocaprioc acid compared to the non-naturally occurring microbial organism without the disruption.

The non-naturally occurring microbial organisms described above, wherein the disruption is a disruption of a rcsA or a homolog thereof.

The non-naturally occurring microbial organisms described above, wherein the disruption is a disruption of a cpsB or a homolog thereof.

The non-naturally occurring microbial organisms described above, wherein the disruption is a disruption of a cpsG or a homolog thereof.

The non-naturally occurring microbial organisms described above, wherein the disruption is a disruption of a cpsBG.

The non-naturally occurring microbial organism described above, further comprising an exogenous nucleic acid encoding a transporter for the 6-aminocaproic acid, wherein the exogenous transporter exports the 6-aminocaproic acid from the cell.

The non-naturally occurring microbial organism described above, wherein the transporter is a YbjE or a homolog thereof.

The non-naturally occurring microbial organisms described above, wherein the transporter is a YhiM.

The non-naturally occurring microbial organisms described above, further comprising a second nucleic acid that encodes a YhiM or a homolog thereof.

The non-naturally occurring microbial organisms described above, wherein the exogenous nucleic acid is chromosomally integrated.

The non-naturally occurring microbial organisms described above, wherein the exogenous nucleic acid is episomal.

The non-naturally occurring microbial organisms described above, wherein the exogenous nucleic acid overexpresses the transporter.

The non-naturally occurring microbial organisms described above, further comprising a disruption of a gene that encodes a transporter wherein the transporter imports 6-aminocaproic acid into the microbial organism.

The non-naturally occurring microbial organisms described above, wherein the disruption is in a gabP or a homolog thereof.

The non-naturally occurring microbial organisms described above, wherein the disruption is in a csiR or a homolog thereof.

The non-naturally occurring microbial organisms described above, further comprising a second disruption in a csiR or a homolog thereof.

The non-naturally occurring microbial organisms described above, further comprising an exogenous nucleic acid encoding a glutamate dehydrogenase.

The non-naturally occurring microbial organisms described above, wherein the glutamate dehydrogenase is a GdhA or a homolog thereof.

The non-naturally occurring microbial organisms described above, wherein the exogenous nucleic acid is chromosomally integrated.

The non-naturally occurring microbial organisms described above, wherein the exogenous nucleic acid is episomal.

The non-naturally occurring microbial organisms described above, wherein the exogenous nucleic acid overexpresses the glutamate dehydrogenase.

In an aspect, the disclosure relates to a non-naturally occurring microbial organism comprising a pathway for making a C6 product wherein the non-naturally occurring microbial organism comprises an exogenous nucleic acid encoding a transporter for the C6 product, wherein the transporter exports the C6 product from the cell.

The non-naturally occurring microbial organism described above, wherein production of the C6 product by the microbial organism is increased compared to the non-naturally occurring microbial organism without the exogenous nucleic acid.

The non-naturally occurring microbial organisms described above, wherein the exogenous nucleic acid is chromosomally integrated.

The non-naturally occurring microbial organisms described above, wherein the exogenous nucleic acid is episomal.

The non-naturally occurring microbial organisms described above, wherein the exogenous nucleic acid overexpresses the transporter.

The non-naturally occurring microbial organisms described above, further comprising a disruption of a gene encoding a transporter that imports C6 product into the microbial organism.

The non-naturally occurring microbial organism of claim 86, further comprising a disruption of a rcsA, a cpsB, a cpsG, or a cpsBG.

The non-naturally occurring microbial organisms described above, further comprising a disruption of a rcsA or a homolog thereof.

The non-naturally occurring microbial organisms described above, further comprising a disruption of a cpsBG.

The non-naturally occurring microbial organisms described above, further comprising a disruption of a cpsB or a homolog thereof.

The non-naturally occurring microbial organisms described above, further comprising a disruption of a cpsG or a homolog thereof.

In an aspect, the disclosure relates to a non-naturally occurring microbial organism comprising a pathway for making a C5-C14 product and an exogenous nucleic acid encoding a transporter for the C5-C14 product, wherein the transporter exports the C5-C14 product from the cell.

The non-naturally occurring microbial organism described above, wherein production of the C5-C14 product by the microbial organism is increased compared to the non-naturally occurring microbial organism without the exogenous nucleic acid.

The non-naturally occurring microbial organism described above, wherein the exogenous nucleic acid is chromosomally integrated.

The non-naturally occurring microbial organisms described above, wherein the exogenous nucleic acid is episomal.

The non-naturally occurring microbial organisms described above, wherein the exogenous nucleic acid overexpresses the transporter.

The non-naturally occurring microbial organisms described above, further comprising a disruption of a gene encoding a transporter that imports C5-C14 product into the microbial organism.

The non-naturally occurring microbial organisms described above, further comprising a disruption of a rcsA, a cpsB, a cpsG, or a cpsBG.

The non-naturally occurring microbial organisms described above, further comprising a disruption of a rcsA or a homolog thereof.

The non-naturally occurring microbial organisms described above, further comprising a disruption of a cpsBG.

The non-naturally occurring microbial organisms described above, further comprising a disruption of a cpsB or a homolog thereof.

The non-naturally occurring microbial organisms described above, further comprising a disruption of a cpsG or a homolog thereof.

The non-naturally occurring microbial organisms described above, wherein production of the 6-aminocaproic acid by the microbial organism is increased compared to the non-naturally occurring microorganism without the exogenous nucleic acid encoding the glutamate dehydrogenase.

In an aspect, the disclosure relates to a method for making a 6-aminocaproic acid, comprising the steps of: providing a non-naturally occurring microbial organism of claim 1; and culturing the non-naturally occurring microbial organism in a medium under conditions where the 6-aminocaproic acid is produced.

The method described above, further comprising the step of transporting the 6-aminocaproic acid from the microbial organism into the medium.

The methods described above, wherein the exogenous nucleic acid is chromosomally integrated.

The methods described above, wherein the exogenous nucleic acid is episomal.

The methods described above, wherein the exogenous nucleic acid overexpresses the transporter.

The methods described above, wherein the exogenous nucleic acid encodes a YbjE or a homolog thereof, or a YhiM or a homolog thereof.

The methods described above, wherein the non-naturally occurring microbial organism further comprising a disruption of a gene encoding a transporter that imports 6-aminocaproic acid into the microbial organism.

The methods described above, wherein the gene is a gabP, or csiR.

The methods described above, wherein the non-naturally occurring microbial organism further comprising an exogenous nucleic acid encoding a glutamate dehydrogenase.

The methods described above, wherein the exogenous nucleic acid encoding the glutamate dehydrogenase is chromosomally integrated.

The methods described above, wherein the exogenous nucleic acid encoding the glutamate dehydrogenase is episomal.

The methods described above, wherein the exogenous nucleic acid encoding the glutamate dehydrogenase overexpresses the glutamate dehydrogenase.

The methods described above, wherein the glutamate dehydrogenase is a GdhA or a homolog thereof.

The methods described above, wherein the non-naturally occurring microbial organism further comprising a disruption of a rcsA, a cpsB, a cpsG, or a cpsBG.

The methods described above, wherein the exogenous transporter is a ybjE, and wherein the non-naturally occurring microbial organism further comprises a disruption of gabP, a disruption of rcsA, and a gene encoding an exogenous glutamate dehydrogenase.

In an aspect, the disclosure relates to a method for making a 6-aminocaproic acid, comprising the steps of: obtaining a non-naturally occurring microbial organism of claim 23; culturing the non-naturally occurring microbial organism in a medium under conditions where the 6-aminocaproic acid is produced.

The method described above, wherein the disrupted gene is a gabP or a csiR.

The methods described above, wherein the non-naturally occurring microbial organism further comprising an exogenous nucleic acid encoding a glutamate dehydrogenase.

The methods described above, wherein the exogenous nucleic acid encoding the glutamate dehydrogenase is chromosomally integrated.

The methods described above, wherein the exogenous nucleic acid encoding the glutamate dehydrogenase is episomal.

The methods described above, wherein the exogenous nucleic acid encoding the glutamate dehydrogenase overexpresses the glutamate dehydrogenase.

The methods described above, wherein the glutamate dehydrogenase is a GdhA.

The methods described above, wherein the non-naturally occurring microbial organism further comprising a disruption of a rcsA, a cpsB, a cpsG, or a cpsBG.

The methods described above, wherein the transporter with the disruption is a gabP, and wherein the non-naturally occurring microbial organism further comprises a disruption of rcsA, and a gene encoding an exogenous glutamate dehydrogenase.

In an aspect, the disclosure relates to a method for making a 6-aminocaproic acid, comprising the steps of: providing a non-naturally occurring microbial organism of claim 38; culturing the non-naturally occurring microbial organism in a medium under conditions where the 6-aminocaproic acid is produced; and transporting the 6-aminocaproic acid from the microbial organism into the medium.

The method described above, wherein the exogenous nucleic acid encoding the glutamate dehydrogenase is chromosomally integrated.

The methods described above, wherein the exogenous nucleic acid encoding the glutamate dehydrogenase is episomal.

The methods described above, wherein the exogenous nucleic acid encoding the glutamate dehydrogenase overexpresses the glutamate dehydrogenase.

The methods described above, wherein the glutamate dehydrogenase is a GdhA or a homolog thereof.

The methods described above, wherein the non-naturally occurring microbial organism further comprising an exogenous nucleic acid encoding a transporter for the 6-aminocaproic acid, wherein the transporter exports the 6-aminocaproic acid from the cell.

The methods described above, wherein the exogenous nucleic acid is chromosomally integrated.

The methods described above, wherein the exogenous nucleic acid is episomal.

The methods described above, wherein the exogenous nucleic acid overexpresses the transporter.

The methods described above, wherein the exogenous nucleic acid encodes a YbjE or a homolog thereof, or a YhiM or a homolog thereof.

The methods described above, wherein the non-naturally occurring microbial organism further comprising a disruption of a transporter that imports 6-aminocaproic acid into the microbial organism.

The methods described above, wherein the disrupted gene is a gabP or a homolog thereof, or a csiR or a homolog thereof.

The methods described above, wherein the non-naturally occurring microbial organism further comprising a disruption of a rcsA, a cpsB, a cpsG, or a cpsBG.

The methods described above, wherein the non-naturally occurring microbial organism further comprises a disruption of gabP, a disruption of rcsA, and an exogenous nucleic acid encoding a YbjE.

In an aspect, the disclosure relates to a method for making a 6-aminocaproic acid, comprising the steps of: providing a non-naturally occurring microbial organism of claim 60; culturing the non-naturally occurring microbial organism in a medium under conditions where the 6-aminocaproic acid is produced; and transporting the 6-aminocaproic acid from the microbial organism into the medium.

The method described above, wherein the non-naturally occurring microbial organism further comprising at least one exogenous nucleic acid encoding a transporter for the 6-aminocaproic acid, wherein the exogenous transporter exports the 6-aminocaproic acid from the cell.

The methods described above, wherein the exogenous nucleic acid is chromosomally integrated.

The methods described above, wherein the exogenous nucleic acid is episomal.

The methods described above, wherein the exogenous nucleic acid overexpresses the transporter.

The methods described above, wherein the exogenous nucleic acid encodes a YbjE or a homolog thereof, or a YhiM or a homolog thereof.

The methods described above, wherein the non-naturally occurring microbial organism further comprising a disruption of a gene encoding a transporter that imports 6-aminocaproic acid into the microbial organism.

The methods described above, wherein the gene is a gabP or a homolog thereof, or a csiR or a homolog thereof.

The methods described above, wherein the non-naturally occurring microbial organism further comprising an exogenous glutamate dehydrogenase.

The methods described above, wherein the exogenous nucleic acid encoding the glutamate dehydrogenase is chromosomally integrated.

The methods described above, wherein the exogenous nucleic acid encoding the glutamate dehydrogenase is episomal.

The methods described above, wherein the exogenous nucleic acid encoding the glutamate dehydrogenase overexpresses the glutamate dehydrogenase.

The methods described above, wherein the glutamate dehydrogenase is a GdhA or a homolog thereof.

In an aspect, the disclosure relates to a method for making C6 product, comprising the steps of: providing a non-naturally occurring microbial organism of claim 82; culturing the non-naturally occurring microbial organism in a medium under conditions where the C6 product is produced; and transporting the C6 product from the microbial organism into the medium.

The method described above, wherein the non-naturally occurring microbial organism further comprising a disruption of a transporter that imports C6 product into the microbial organism.

The methods described above, wherein the non-naturally occurring microbial organism further comprising a disruption of a rcsA, a cpsB, a cpsG, or a cpsBG.

In an aspect, the disclosure relates to a method for making C5-C14 product, comprising the steps of: providing a non-naturally occurring microbial organism of claim 93; culturing the non-naturally occurring microbial organism in a medium under conditions where the C5-C14 product is produced; and transporting the C5-C14 product from the microbial organism into the medium.

The method described above, wherein the non-naturally occurring microbial organism further comprising a disruption of a transporter that imports C6 product into the microbial organism.

The methods described above, wherein the non-naturally occurring microbial organism further comprising a disruption of a rcsA, cpsB, cpsG, or a cpsBG.

### BRIEF DESCRIPTION OF THE DRAWINGS

FIG. 1 shows exemplary pathways from succinyl-CoA and acetyl-CoA to 6-aminocaproate, hexamethylenediamine (HMDA), and caprolactam. The enzymes are designated as follows: A) 3-oxoadipyl-CoA thiolase, B) 3-oxoadipyl-CoA reductase, C) 3-hydroxyadipyl-CoA dehydratase, D) 5-carboxy-2-pentenoyl-CoA reductase, E) 3-oxoadipyl-CoA/acyl-CoA transferase, F) 3-oxoadipyl-CoA synthase, G) 3-oxoadipyl-CoA hydrolase, H) 3-oxoadipate reductase, I) 3-hydroxyadipate dehydratase, J) 5-carboxy-2-pentenoate reductase, K) adipyl-CoA/acyl-CoA transferase, L) adipyl-CoA synthase, M) adipyl-CoA hydrolase, N) adipyl-CoA reductase (aldehyde forming), 0) 6-aminocaproate transaminase, P) 6-aminocaproate dehydrogenase, Q) 6-aminocaproyl-CoA/acyl-CoA transferase, R) 6-aminocaproyl-CoA synthase, S) amidohydrolase, T) spontaneous cyclization, U) 6-aminocaproyl-CoA reductase (aldehyde forming), V) HMDA transaminase, W) HMDA dehydrogenase, X) adipate reductase, Y) adipate kinase, Z) adipylphosphate reductase.

FIG. 2A shows an exemplary pathway from succinyl-CoA and acetyl-CoA to exported 6-aminocaproic acid. FIGS. 2B and 2C show a bar chart and a graph, respectively for the production of 6-aminocaproic acid with the expression of exogenous glutamate dehydrogenase. In FIG. 2B, the first bar from the left is control; second bar from left is lof GDH expression; third bar from the left is medium expression of GDH; and fourth bar from the left is high expression of GDH. In FIG. 2C, the upper line is medium GDH expression; the middle line is high GDH expression; and the lowest line is control.

FIG. 3 shows an exemplary pathway for synthesis of 6-amino caproic acid and adipate using lysine as a starting point.

FIG. 4 shows an exemplary caprolactam synthesis pathway using adipyl-CoA as a starting point.

FIG. 5 shows shows exemplary pathways to 6-aminocaproate from pyruvate and succinic semialdehyde. Enzymes are A) HODH aldolase, B) OHED hydratase, C) OHED reductase, D) 2-OHD decarboxylase, E) adipate semialdehyde aminotransferase and/or adipate semialdehyde oxidoreductase (aminating), F) OHED decarboxylase, G) 6-OHE reductase, H) 2-OHD aminotransferase and/or 2-OHD oxidoreductase (aminating),I) 2-AHD decarboxylase, J) OHED aminotransferase and/or OHED oxidoreductase (aminating), K) 2-AHE reductase, L) HODH formate-lyase and/or HODH dehydrogenase, M) 3-hydroxyadipyl-CoA dehydratase, N) 2,3-dehydroadipyl-CoA reductase, O) adipyl-CoA dehydrogenase, P) OHED formate-lyase and/or OHED dehydrogenase, Q) 2-OHD formate-lyase and/or 2-OHD dehydrogenase. Abbreviations are: HODH=4-hydroxy-2-oxoheptane-1,7-dioate, OHED=2-oxohept-4-ene-1,7-dioate, 2-OHD=2-oxoheptane-1,7-dioate, 2-AHE=2-aminohept-4-ene-1,7-dioate, 2-AHD=2-aminoheptane-1,7-dioate, and 6-OHE=6-oxohex-4-enoate.

FIG. 6 shows exemplary pathways to hexamethylenediamine from 6-aminocapropate. Enzymes are A) 6-aminocaproate kinase, B) 6-AHOP oxidoreductase, C) 6-aminocaproic semialdehyde aminotransferase and/or 6-aminocaproic semialdehyde oxidoreductase (aminating), D) 6-aminocaproate N-acetyltransferase, E) 6-acetamidohexanoate kinase, F) 6-AAHOP oxidoreductase, G) 6-acetamidohexanal aminotransferase and/or 6-acetamidohexanal oxidoreductase (aminating), H) 6-acetamidohexanamine N-acetyltransferase and/or 6-acetamidohexanamine hydrolase (amide), I) 6-acetamidohexanoate CoA transferase and/or 6-acetamidohexanoate CoA ligase, J) 6-acetamidohexanoyl-CoA oxidoreductase, K) 6-AAHOP acyltransferase, L) 6-AHOP acyltransferase, M) 6-aminocaproate CoA transferase and/or 6-aminocaproate CoA ligase, N) 6-aminocaproyl-CoA oxidoreductase. Abbreviations are: 6-AAHOP=[(6-acetamidohexanoyl)oxy]phosphonate and 6-AHOP=[(6-aminohexanoyl)oxy]phosphonate.

FIG. 7 shows exemplary biosynthetic pathways leading to 1,6-hexanediol. A) is a 6-aminocaproyl-CoA transferase or synthetase catalyzing conversion of 6ACA to 6-aminocaproyl-CoA; B) is a 6-aminocaproyl-CoA reductase catalyzing conversion of 6-aminocaproyl-CoA to 6-aminocaproate semialdehyde; C) is a 6-aminocaproate semialdehyde reductase catalyzing conversion of 6-aminocaproate semialdehyde to 6-aminohexanol; D) is a 6-aminocaproate reductase catalyzing conversion of 6ACA to 6-aminocaproate semialdehyde; E) is an adipyl-CoA reductase adipyl-CoA to adipate semialdehyde; F) is an adipate semialdehyde reductase catalyzing conversion of adipate semialdehyde to 6-hydroxyhexanoate; G) is a 6-hydroxyhexanoyl-CoA transferase or synthetase catalyzing conversion of 6-hydroxyhexanoate to 6-hydroxyhexanoyl-CoA; H) is a 6-hydroxyhexanoyl-CoA reductase catalyzing conversion of 6-hydroxyhexanoyl-CoA to 6-hydroxyhexanal; I) is a 6-hydroxyhexanal reductase catalyzing conversion of 6-hydroxyhexanal to HDO; J) is a 6-aminohexanol aminotransferase or oxidoreductases catalyzing conversion of 6-aminohexanol to 6-hydroxyhexanal; K) is a 6-hydroxyhexanoate reductase catalyzing conversion of 6-hydroxyhexanoate to 6-hydroxyhexanal; L) is an adipate reductase catalyzing conversion of ADA to adipate semialdehyde; and M) is an adipyl-CoA transferase, hydrolase or synthase catalyzing conversion of adipyl-CoA to ADA.

FIG. 8 shows exemplary pathways from adipate or adipyl-CoA to caprolactone. Enzymes are A. adipyl-CoA reductase, B. adipate semialdehyde reductase, C. 6-hydroxyhexanoyl-CoA transferase or synthetase, D. 6-hydroxyhexanoyl-CoA cyclase or spontaneous cyclization, E. adipate reductase, F. adipyl-CoA transferase, synthetase or hydrolase, G. 6-hydroxyhexanoate cyclase, H. 6-hydroxyhexanoate kinase, I. 6-hydroxyhexanoyl phosphate cyclase or spontaneous cyclization, J. phosphotrans-6-hydroxyhexanoylase.

FIG. 9A shows the ratio of byproducts in a strain with AspeAB compared to a strain with wild-type speAB. FIG. 9B shows the titer of byproducts in a strain with AspeAB compared to a strain with wild-type speAB.

### DETAILED DESCRIPTION

It is to be understood that the teachings of this disclosure are not limited to the particular embodiments described, and as such can, of course, vary. It is also to be understood that the terminology used herein is for the purpose of describing particular exemplifications only, and is not intended to be limiting, since the scope of the present teachings will be limited only by the appended claims.

Unless defined otherwise, all technical and scientific terms used herein have the same meaning as commonly understood by those of ordinary skill in the art. Any methods, devices and materials similar or equivalent to those described herein can be used in the practice of the invention. The following definitions are provided to facilitate understanding of certain terms used frequently herein and are not meant to limit the scope of the present disclosure. All documents (e. g. patent applications or patents) referred to herein are incorporated by reference in their entirety.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 g, it is intended that the concentration be understood to be at least approximately or about 10 g.

As will be apparent to those of skill in the art upon reading this disclosure, each of the individual exemplifications described and illustrated herein has discrete components and features which can be readily separated from or combined with the features of any of the other several exemplifications without departing from the scope or spirit of the present teachings. Any recited method can be carried out in the order of events recited or in any other order which is logically possible.

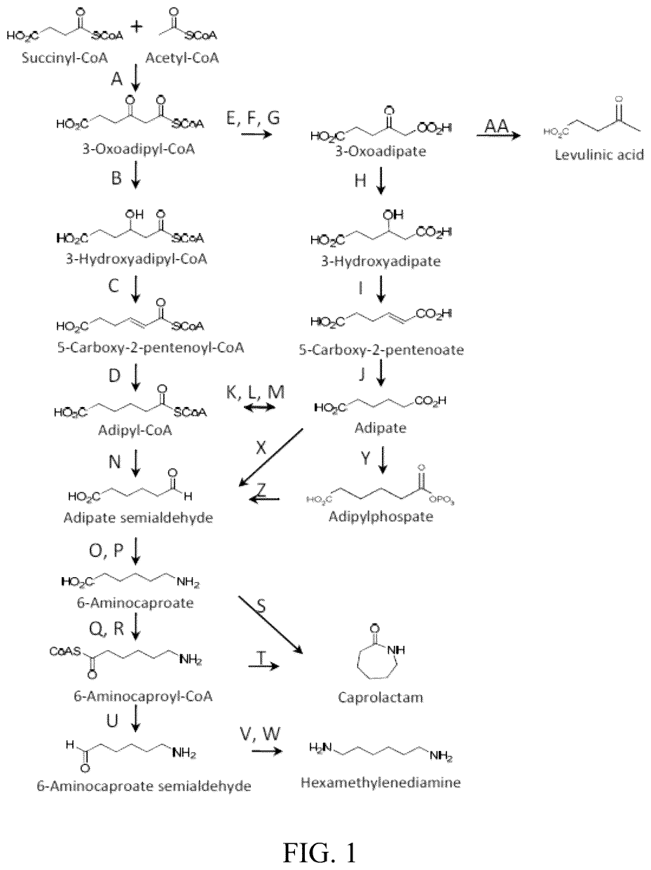

Exemplary amide compounds, e.g., nylon intermediates, can be biosynthesized using the pathway described in FIG. 1. The FIG. 1 pathway can be provided in a genetically modified cell such as those described herein (e. g., a non-naturally occurring microorganism). The engineered cell can include at least one exogenous nucleic acid encoding a pathway enzyme expressed in a sufficient amount to produce 6-aminocaproic acid, caprolactam, and/or hexamethylenediamine.

The engineered pathway can be an HMD pathway as set forth in FIG. 1. The HMD pathway can be provided in a genetically modified cell described herein (e. g., a non-naturally occurring microorganism) where the HMD pathway includes at least one exogenous nucleic acid encoding a HMD pathway enzyme expressed in a sufficient amount to produce HMD. The enzymes can include 1A is a 3-oxoadipyl-CoA thiolase; 1B is a 3-oxoadipyl-CoA reductransaminasee; 1C is a 3-hydroxyadipyl-CoA dehydratransaminasee; 1D is adipate semialdehydereductransaminasee; 1E is a 3-oxoadipyl-CoA/acyl-CoA transferase; 1F is a 3-oxoadipyl-CoA synthase; 1G is a 3-oxoadipyl-CoA hydrolase; 1H is a 3-oxoadipate reductransaminasee; 1I is a 3-hydroxyadipate dehydratransaminasee; 1J is a 5-carboxy-2-pentenoate reductransaminasee; 1K is an adipyl-CoA/acyl-CoA transferase; 1L is an adipyl-CoA synthase; 1M is an adipyl-CoA hydrolase; 1N is an adipyl-CoA reductransaminasee (aldehyde forming); 1O is a 6-aminocaproate transaminase; 1P is a 6-aminocaproate dehydrogenase; 1Q is a 6-aminocaproyl-CoA/acyl-CoA transferase; 1R is a 6-aminocaproyl-CoA synthase; 1S is an amidohydrolase; 1T is spontaneous cyclization; 1U is a 6-aminocaproyl-CoA reductransaminasee (aldehyde forming); 1V is a HMDA transaminase; and 1W is a HMDA dehydrogenase.

With reference to FIG. 1, the non-naturally occurring microorganism can have one or more of the following pathways: ABCDNOPQRUVW; ABCDNOPQRT; or: ABCDNOPS. Other exemplary pathways can produce adipate semialdehyde include those described in U.S. Pat. No. 8,377,680 incorporated herein by reference in its entirety.

FIG. 1 also shows a pathway from 6-aminocaproate to 6-aminocaproyl-CoA by a transferase or synthase enzyme (FIG. 1, Step Q or R) followed by the spontaneous cyclization of 6-aminocaproyl-CoA to form caprolactam (FIG. 1, Step T). 6-aminocaproate can also be activated to 6-aminocaproyl-CoA (FIG. 1, Step Q or R), followed by a reduction (FIG. 1, Step U) and amination (FIG. 1, Step V or W) to form HMDA. 6-Aminocaproic acid can be activated to 6-aminocaproyl-phosphate instead of 6-aminocaproyl-CoA. 6-Aminocaproyl-phosphate can spontaneously cyclize to form caprolactam. 6-aminocaproyl-phosphate can be reduced to 6-aminocaproate semialdehyde, which can be then converted to HMDA as depicted in FIG. 1.

Non-naturally occurring microbial organisms described here in can include engineering of transporters including, for example, engineering the microbial organism to increase export of a desired product. A microbial organism can also be engineered to decrease the importation of a desired product. Such engineering of the transporters in a microbial organism can increase the production of the desired product from the microbial organism. The exporting (or secretion) of the desired product from the microbial organism and/or inhibition of importation of the desired product unto the microbial organism can enhance product formation by lowering the concentration of product in the microbial organism allowing more reactants in the microbial cell to become products. Production of a desired product can also be increased by increasing the importation (and/or reducing the export) of reactants and/or intermediates for the desired product. Production of a desired product can also be increased by increasing the importation (and/or reducing the export) of products that are made from the desired product (products for which the desired product is a reactant or an intermediate). Production of a desired product can be increased by reducing the production of intermediates and products that compete for carbon with the pathway making the desired product.

As described below in Example 2, the production of 6-aminocaproic acid is limited by secretion/export of 6-aminocaproic acid from the microbial organism. When the microbial organism is engineered to increase export (secretion) of 6-aminocaproic acid, the amount of 6-aminocaproic acid obtained per cell unit from the microbial organism is increased. For example, wheren the microbial organism is engineered to express the 6-aminocaproic acid exporters lysO (aka ybjE) and/or yhiM, from E. coli, the amount of 6-aminocaproic acid is increased. Similarly, when the microbial organism is engineered to inhibit 6-aminocaproic acid importers the production of 6-aminocaproic acid is also increased. For example, when the 6-aminocaproic acid importers gabP or homologs thereof and/or csiR or homologs thereof, from E. coli, are disrupted the production of 6-aminocaproic acid is increased. The disruption can be made in a nucleic acid having the sequence of SEQ ID NO: 5, or a nucleic acid sequence having 99%, 95%, 90%, 80% or 70% homology to SEQ ID NO:5. The disruption can also be made in a nucleic acid encoding the amino acid sequence of SEQ ID NO: 6, or an amino acid sequence having 99%, 95%, 90%, 80% or 70% homology to SEQ ID NO: 6. The disruption can be made in a nucleic acid having the sequence of SEQ ID NO: 7, or a nucleic acid sequence having 99%, 95%, 90%, 80% or 70% homology to SEQ ID NO: 7. The disruption can also be made in a nucleic acid encoding the amino acid sequence of SEQ ID NO: 8, or an amino acid sequence having 99%, 95%, 90%, 80% or 70% homology to SEQ ID NO: 8.

In an aspect, the engineered microbial organism for making 6-aminocaproic acid can overexpress lysO (aka ybjE) or homologs thereof, and/or yhiM or homologs thereof, and/or one or more of the appropriate enzymes in Table 16 below, and/or have disruptions of gabP or homologs thereof, and/or csiR or homologs thereof. In another aspect, the engineered microbial organism for making 6-aminocaproic acid can overexpress one or more of the appropriate enzymes in Table 16 below including, for example, a nucleic acid of SEQ ID NO: 1, 17 (acc. #P75826), 19 (acc. #A0A3S6EWD1), 21 (acc. #A0A3R0JPG3), 23 (acc. #A0A2X5EV87), 25 (acc. #A0A0B6FGQ0), 27 (acc. #A0A0T9T4V6), 29 (acc. #A0A3X9TWR2), 31 (acc. #A0A0Q4NI65), 57 (acc. #A0A447V4H2), 59 (acc. #A0A085HLU7), 61 (acc. #A0A085AG20), 63 (acc. #A0A2X2DZ65), 65 (UniParc ID UPI00045BA014), 67 (acc. #A0A1B9PQG6), 69 (acc. #A0A0Q9CPY2), 71 (acc. #A0A2N5KTP3), 73 (acc. #A0A2U3BBA9), 75 (acc. #A0A3S0AXG7), 77 (acc. #A0A198FET8), 79 (acc. #A0A1C5WGH0), 81 (acc. #A0A3E4Z618), 83 (acc. #A0A0B6XA16), 85 (acc. #A0A0G3CKY0), 87 (acc. #A0A2N4W2H6), 89 (acc. #A0A101K111), 91 (acc. #A0A356QXL1), and/or 93 (acc. #W3V0I2), or one or more nucleic acid sequence having 99%, 95%, 90%, 80% or 70% identity to SEQ ID NO: 1, 17, 19, 21, 23, 25, 27, 29, 31, 57, 59, 61, 63, 65, 67, 69, 71, 73, 75, 77, 79, 81, 83, 85, 87, 89, 91, 93, and/or SEQ ID NO: 3 or a nucleic acid sequence having 99%, 95%, 90%, 80% or 70% homology to SEQ ID NO: 3, and/or have disruptions of SEQ ID NO: 5 or a nucleic acid sequence having 99%, 95%, 90%, 80% or 70% identity to SEQ ID NO: 5, and/or SEQ ID NO: 7 or a nucleic acid sequence having 99%, 95%, 90%, 80% or 70% identity to SEQ ID NO: 7. In another aspect, the engineered microbial organism for making 6-aminocaproic acid can overexpress a nucleic acid encoding one or more of SEQ ID NO: 2, 4, 18, 20, 22, 24, 26, 28, 30, 32, 58, 60, 62, 64, 66, 68, 70, 72, 74, 76, 78, 80, 82, 84, 86, 88, 90, 92, and/or 94, or one or more polypeptide having 99%, 95%, 90%, 80% or 70% identity to SEQ ID NO: 2, 4, 18, 20, 22, 24, 26, 28, 30, 32, 58, 60, 62, 64, 66, 68, 70, 72, 74, 76, 78, 80, 82, 84, 86, 88, 90, 92, and/or 94, and/or have disruptions of a nucleic acid encoding SEQ ID NO: 6 or a polypeptide having 99%, 95%, 90%, 80% or 70% identity to SEQ ID NO: 6, and/or SEQ ID NO: 8 or a polypeptide having 99%, 95%, 90%, 80% or 70% identity to SEQ ID NO: 8.

Other importers that can be engineered into a microbial organism for increasing the synthesis of other desired products include, for example, solute carrier (SLC) transporters. SLCs can be membrane proteins that transport solutes (ions, metabolites, peptides, drugs, ligands, other organic small molecules, etc.) across membranes. SLCs can be active transporters and utilize energy (e.g., ATP or an ion gradient) to transport a solute (e.g., ligand) into the microbial organism. SLCs can be passive transporters that do not utilize energy for transport of the solute (e.g., ligand). Exemplary SLCs are described in, for example, Fath et al, Microbiol. Rev. 57:995-1017 (1993); Moussatova et al, Biochim. Biopys. Acta Biomemb. 9:1757-1771 (2008); Lin et al., Nat. Rev. Drug Discov. 14:543-560 (2015); Hediger et al., Mol. Aspects Med. 34:95-107 (2013); Ye et al., PLoS ONE 9:e88883 (2014); Schlessinger et al., Curr. Top. Med. Chem. 13:843-856 (2013); Saier, Microbiol. 146:1775-1795 (2000); SLC Tables at slc.bioparadigms.org; HUGO Gene Nomenclature for SLCs at genenames.org/cgi-bin/genefamilies/set/752, all of which are incorporated by reference in their entirety for all purposes. Included within SLCs are, for example, channels, pores, electrochemical potential driven transporters, primary active transporters, group translocators, electron carriers, ATP powered pumps, ion channels, and transporters, including uniporters, symporters, and antiporters.

The production of amine compounds that involve a transaminase in the synthesis pathway can be increased by increasing the activity of glutamate dehydrogenase. Transaminases that utilize glutamic acid to obtain an amino group for the amine product and produce α-ketoglutarate from the glutamic acid can increase the production of product by increasing the expression (and/or activity) of glutamate dehydrogenase (“GDH”). Glutamate dehydrogenase has a large negative Gibbs free energy for making glutamate from α-ketoglutarate and NH4, and so, adding GDH to a microbial cell can produce an excess of glutamate (large amount of this reactant) to react with the transaminase and the unaminated intermediate. Transporters for ammonium can also be used to increase the production of product from the transaminase. By increasing the activity of importers of ammonium, and/or decreasing the activity of exporters of ammonium, the intracellular concentration of this reactant for GDH can be increased which will further increase the production of glutamate by the GDH which further increased the production of product by the transaminase.

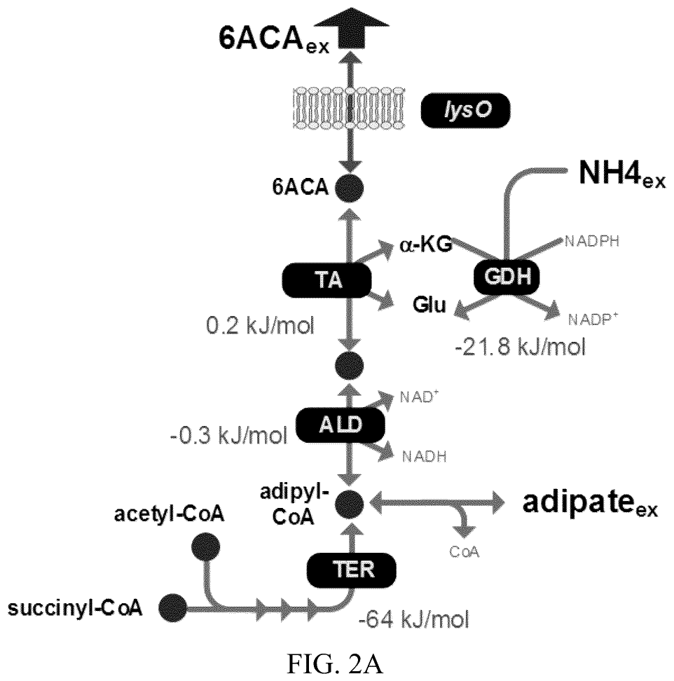

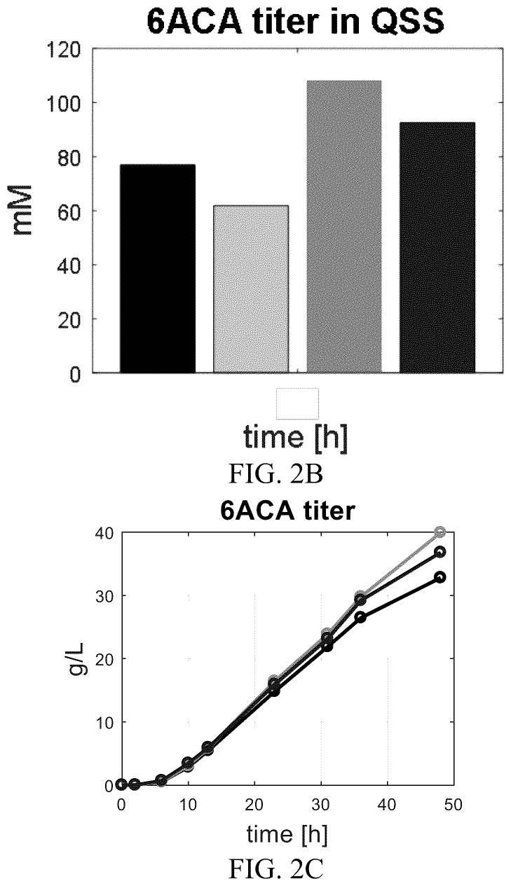

For example, as shown in Examples 5 and 9 below, the overexpression of GDH (e.g., gdhA or homologs thereof) increases production of 6-aminocaproic acid. The expressed GDH can also be encoded by one or more of the enzymes from Example 5 or Table 17 below including, for example, one or more of SEQ ID NO: 9, 33 (acc. #A0A1F9IMB6), 35 (acc. #C7RFH9), 37 (acc. #A0A3M1CG83), 39 (acc. #A0A095X4D3), 41 (acc. #A0A2S7L1V8), 43 (acc. #W5WWS1), 45 (acc. #P94316), 47 (acc. #AAA25611), 53 (acc. #A0A1V4WK45), and/or 55 (acc. #a0A367ZGM1), or one or more nucleic acid sequence having 99%, 95%, 90%, 80% or 70% identity to SEQ ID NO:9, 33, 35, 37, 39, 41, 43, 45, 47, 53, and/or 55. The expressed GDH can also be expressed from one or more nucleic acid encoding one or more amino acid sequence of SEQ ID NO: 10, 34, 36, 38, 40, 42, 44, 46, 48, 54, and/or 56, or one or more amino acid sequence having 99%, 95%, 90%, 80% or 70% identity to SEQ ID NO:10, 34, 36, 38, 40, 42, 44, 46, 48, 54, and/or 56. FIG. 2A shows that the GDH produces an excess of glutamate increasing the concentration of this reactant and driving the transaminase reaction towards 6-aminocaproic acid. As show in FIG. 2A, the Gibbs free energy for transaminase reaction forming 6-aminocaproic acid is close to zero, and so reactants and products (6-aminocaproic acid) are present in close to equal amounts. The high negative Gibbs free energy for GDH production of glutamate means that GDH produces a large excess of product (glutamate) compared to the reactants (α-ketoglutarate and ammonium). The excess of glutamate can drive the transaminase reaction towards 6-aminocaproic acid increasing the amount of this desired product. Glutamate production can also be increased by overexpressing, for example, the ammonium transporters amtB from E. coli, the W148L variant of amt B from E. coli, and/or amtA from C. glutamicum. These ammonium transporters can increase the ammonium concentration in the microbial organism driving the GDH reaction to produce more glutamate.

The production of desired products can also be increased by eliminating the mucoid phenotype from the microbial organism. Microbial organisms with the mucoid phenotype produce extracellular polysaccharides which for some microbial organisms can come to represent a large amount of the cellular carbon. The mucoid phenotype is associated with escape from immune-surveillance and the formation of biofilms that are advantageous for the microbial organism in a host of situations. The mucoid phenotype is also associated with a number of characteristics that are deleterious for the manufacture of desired products. For example, microbial organisms with the mucoid phenotype pellet poorly and do not behave well and reproducibly in manufacturing cultures. These deleterious characteristics can reduce the production of desired products from the microbial organism. Engineering a microbial organism to eliminated or reduce/inhibit the mucoid phenotype can relieve these productions issues and free the carbon used to make the extracellular polysaccharides for the production of desired products, and so, increase the production of these desired products.

For example, the disruption of rcsA or homologs thereof, rcsB or homologs thereof, wcaF or homologs thereof, and/or cpsB or homologs thereof, and/or cpsG or homologs thereof, and/or cpsBG or homologs thereof knocked out the mucoid phenotype and made microbial organisms that were non-mucoid. The disruption can be made in a nucleic acid having the sequence of SEQ ID NO: 11, or a nucleic acid sequence having 99%, 95%, 90%, 80% or 70% identity to SEQ ID NO: 11. The disruption can also be made in a nucleic acid encoding the amino acid sequence of SEQ ID NO: 12, or an amino acid sequence having 99%, 95%, 90%, 80% or 70% identity to SEQ ID NO:12. The disruption can be made in a nucleic acid having the sequence of SEQ ID NO: 13, or a nucleic acid sequence having 99%, 95%, 90%, 80% or 70% identity to SEQ ID NO:13. The disruption can also be made in a nucleic acid encoding the amino acid sequence of SEQ ID NO: 14, or an amino acid sequence having 99%, 95%, 90%, 80% or 70% identity to SEQ ID NO:14. The disruption can be made in a nucleic acid having the sequence of SEQ ID NO: 15, or a nucleic acid sequence having 99%, 95%, 90%, 80% or 70% identity to SEQ ID NO:15. The disruption can also be made in a nucleic acid encoding the amino acid sequence of SEQ ID NO: 16, or an amino acid sequence having 99%, 95%, 90%, 80% or 70% identity to SEQ ID NO:16. The disruption of rcsA and/or cpsBG also markedly increased the production of desired products (see Example 6), whereas the disruption of rcsB or wcaF did not increase production of a desired product. Microbial organisms with rcsA or cpsBG disruptions produced 3-4 times more 6-aminocaproic acid than the mucoid parent strain (or the strain with disrupted rcsB or wcaF).

The production of desired product can also be increased by reducing the amount of competiting intermediate and/or products that are made. If adapic acid (ADA) is byproduct in a production cell, the amount of desired product (e.g., 6-aminocaproic acid, caprolactam, and/or hexamethylenediol) can be increased by disrupting gabD (succinate-semialdehyde dehydrogenase NADP), sad (succinate-semialdehyde dehydrogenase NAD), and/or ybfF (acyl-CoA esterase). If 6-hydroxycaproic acid (6HCA) is a byproduct, the amount of desired product (e.g., 6-aminocaproic acid, caprolactam, and/or hexamethylenediol) can be increased by disrupting yghD (Type II secretion system protein), yjgB (alcohol dehydrogenase), and/or yahK (aldehyde reductase). If gamma amino butyric acid (GABA) is byproduct, the amount of desired product (e.g., 6-aminocaproic acid, caprolactam, and/or hexamethylenediol) can be increased by disrupting gabT (4-aminobutyrate aminotransferase).

Table 1 below lists genes, DNA sequences, protein sequences, accession numbers, and locus tags.

TABLE 1

| | | | | Locus | |
|---|---|---|---|---|---|
| Gene | DNA | Protein | Accession # | Tag | EC No. |
| ybjE | SEQ ID NO: 1 | SEQ ID NO: 2 | NP_415395.2 | b0874 | |
| yhiM | SEQ ID NO: 3 | SEQ ID NO: 4 | NP_417948.4 | b3491 | |
| gabP | SEQ ID NO: 5 | SEQ ID NO: 6 | NP_417149.1 | b2663 | |
| csiR | SEQ ID NO: 7 | SEQ ID NO: 8 | NP_417150.4 | b2664 | |
| gdhA | SEQ ID NO: 9 | SEQ ID NO: 10 | NP_416275.1 | b1761 | 1.4.1.4 |
| rcsA | SEQ ID NO: 11 | SEQ ID NO: 12 | NP_416461.1 | b1951 | |
| cpsB | SEQ ID NO: 13 | SEQ ID NO: 14 | NP_416553.1 | b2049 | 2.7.7.13 or |
| | | | | | 2.7.7.22 |
| cpsG | SEQ ID NO: 15 | SEQ ID NO: 16 | NP_416552.1 | b2048 | 5.4.2.8 |

Representative homologs for ybjE include, for example, those in Table 2 below:

TABLE 2
ybjE Homologs

| identity | accession | identity | accession | identity | accession |
|---|---|---|---|---|---|
| 99.7 | NP_308987.1 | 62.5 | WP_004235793.1 | 52.5 | WP_009387562.1 |
| 99.7 | NP_706755.1 | 62.5 | WP_045969927.1 | 51.5 | WP_011261557.1 |
| 99.3 | WP_000491144.1 | 61.9 | WP_038238030.1 | 52.5 | WP_068899709.1 |
| 91 | WP_012133055.1 | 61.5 | WP_027274488.1 | 54.5 | WP_065577074.1 |
| 90.6 | WP_012905211.1 | 62.5 | WP_021325224.1 | 51.3 | WP_021818509.1 |
| 89.3 | WP_000491115.1 | 63.2 | WP_011145886.1 | 50.2 | WP_005505937.1 |
| 90.3 | WP_060569860.1 | 62.9 | WP_010846887.1 | 50.5 | WP_043511721.1 |
| 89 | NP_459915.1 | 65.1 | WP_025422823.1 | 50.7 | WP_075370192.1 |
| 88.3 | WP_000490988.1 | 59.5 | WP_053908957.1 | 50.5 | WP_009096647.1 |
| 87.3 | WP_013366893.1 | 60.2 | WP_025902875.1 | | |
| 85.3 | WP_038154735.1 | 60.2 | WP_068442074.1 | | |
| 83.9 | WP_023481506.1 | 58.9 | WP_008915178.1 | | |
| 82.9 | WP_004147773.1 | 60.7 | WP_066752185.1 | | |
| 82.9 | WP_004147773.1 | 59.5 | WP_012987464.1 | | |
| 82.3 | WP_002463118.1 | 58.1 | WP_010863822.1 | | |
| 82.3 | WP_034494287.1 | 55.5 | WP_026823309.1 | | |
| 81.6 | WP_048278507.1 | 54.5 | WP_061000073.1 | | |
| 79.6 | WP_061282833.1 | 52.9 | WP_046222493.1 | | |
| 79.9 | WP_016535588.1 | 53.5 | NP_797618.1 | | |
| 76.5 | WP_036107695.1 | 53.5 | WP_047883408.1 | | |
| 73.9 | WP_002439504.1 | 54.2 | WP_044623796.1 | | |
| 74.6 | WP_052896562.1 | 51.8 | WP_011705573.1 | | |
| 84.8 | WP_002896432.1 | 53.5 | WP_011150093.1 | | |
| 70.8 | WP_004965015.1 | 54.5 | WP_007467167.1 | | |
| 71.8 | WP_002211358.1 | 53.5 | WP_004395038.1 | | |
| 69.6 | WP_013201506.1 | 51.9 | WP_017054533.1 | | |
| 70.8 | WP_024911886.1 | 51.9 | WP_026025698.1 | | |
| 69.9 | WP_034789100.1 | 54.2 | WP_005368690.1 | | |
| 69.5 | WP_061798102.1 | 52.5 | NP_230796.1 | | |
| 70.1 | WP_076942281.1 | 53.2 | WP_021703678.1 | | |
| 68.6 | WP_025800939.1 | 53.2 | WP_014293203.1 | | |
| 67.6 | WP_029093558.1 | 53.5 | WP_053395635.1 | | |
| 68.9 | WP_048639232.1 | 51.2 | WP_012604234.1 | | |
| 70.8 | WP_023491948.1 | 52.2 | WP_047872347.1 | | |
| 70 | WP_021015570.1 | 53.2 | WP_062709055.1 | | |
| 67.2 | WP_005287641.1 | 53.2 | WP_046075447.1 | | |
| 66.2 | WP_047781379.1 | 52.5 | WP_068328819.1 | | |
| 65.9 | WP_034941358.1 | 53.4 | WP_071471192.1 | | |
| 65.6 | WP_011094204.1 | 51.5 | WP_005298535.1 | | |
| 67 | WP_026743441.1 | 50.8 | WP_068698802.1 | | |

Representative homologs for yhiM include, for example, those in Table 3 below:

TABLE 3
yhiM Homologs

| identity | accession | identity | accession | identity | accession |
|---|---|---|---|---|---|
| 99.714 | CSR38991.1 | 98.209 | EGI90439.1 | 55.241 | WP_088860944.1 |
| 99.694 | WP_146758915.1 | 97.765 | CSQ64563.1 | 54.7 | WP_012697359.1 |
| 99.691 | WP_000894161.1 | 96.736 | WP_134799151.1 | 54.674 | WP_026215110.1 |
| 99.676 | OYI09881.1 | 91.589 | WP_117031587.1 | 54.572 | WP_091089750.1 |
| 99.611 | CSQ73132.1 | 91.257 | EBV5602406.1 | 54 | WP_150920967.1 |
| 99.593 | OCY87936.1 | 90.769 | EAB7041734.1 | 53.736 | WP_148035678.1 |
| 99.429 | WP_154813013.1 | 90.571 | WP_001318089.1 | 53.429 | WP_150924208.1 |
| 99.383 | SRV86132.1 | 90.351 | EAA2490775.1 | 53.276 | WP_135064867.1 |
| 99.143 | MLU14817.1 | 90 | WP_016158897.1 | 53.143 | CAA2141987.1 |
| 98.857 | OYI41353.1 | 89.429 | WP_001517283.1 | 52.571 | WP_092867765.1 |
| 98.806 | ODG74425.1 | 88.136 | EBO3310906.1 | 52.436 | WP_135116030.1 |
| 98.592 | EAA0484737.1 | 58 | WP_012417708.1 | 52 | WP_068625586.1 |
| 98.585 | MJQ67314.1 | 58 | WP_114851959.1 | 51.714 | WP_056647755.1 |
| 98.571 | WP_134796546.1 | 56.571 | WP_150973537.1 | 51.6 | WP_056647755.1 |
| 98.544 | EFW54220.1 | 56.286 | WP_125946421.1 | 50.852 | WP_066586210.1 |
| 98.492 | EIQ04384.1 | 56 | RSV14362.1 | 50.6 | WP_055240933.1 |
| 98.286 | WP_001028889.1 | 55.714 | WP_111069755.1 | 50.571 | WP_147722741.1 |
| 98.22 | EGJ00821.1 | 55.714 | WP_123833051.1 | | |

Representative homologs for gabP include, for example, those in Table 4 below:

TABLE 4
gabP Homologs

| identity | accession | identity | accession | identity | accession |
|---|---|---|---|---|---|
| 99.8 | NP_311551.1 | 66.1 | WP_059316629.1 | 57.8 | WP_048278073.1 |
| 99.8 | WP_001301367.1 | 64.1 | WP_052355144.1 | 54 | WP_014206843.1 |
| 93.3 | WP_002889795.1 | 64.5 | WP_035524296.1 | 52.9 | WP_001058165.1 |
| 92.9 | WP_042288477.1 | 65.9 | WP_068367177.1 | 53.6 | WP_031091988.1 |
| 92.1 | NP_461719.1 | 64.9 | WP_006400482.1 | 56.7 | WP_073353772.1 |
| 91.6 | WP_000531293.1 | 65 | WP_010796658.1 | 55 | WP_046740863.1 |
| 91.2 | WP_048253967.1 | 64.3 | WP_010798818.1 | 57.7 | WP_043861690.1 |
| 89.2 | WP_013364901.1 | 65.9 | WP_038714857.1 | 52.8 | WP_016655647.1 |
| 89.1 | WP_002442794.1 | 65.4 | WP_047965892.1 | 56.2 | WP_019957986.1 |
| 83.3 | WP_025801404.1 | 60.4 | NP_746226.1 | 53.2 | WP_056785610.1 |
| 71.1 | NP_745055.1 | 61.9 | XP_007453024.1 | 53.3 | WP_073723199.1 |
| 71.6 | WP_043257541.1 | 61.3 | WP_060710433.1 | 53.2 | WP_005026631.1 |
| 67.5 | WP_045484559.1 | 62.7 | WP_068103575.1 | 52.8 | WP_028415199.1 |
| 68.7 | WP_017132610.1 | 64.1 | WP_045194817.1 | 52.5 | WP_073921263.1 |
| 68.4 | WP_009396069.1 | 61.9 | WP_042574174.1 | 54.8 | WP_009454829.1 |
| 67.1 | NP_795086.1 | 59.8 | WP_038414123.1 | 52.9 | WP_014156273.1 |
| 66.9 | WP_008367864.1 | 58.7 | WP_045489171.1 | 53.4 | WP_046263708.1 |
| 67.2 | WP_048383922.1 | 61.7 | WP_054290263.1 | 54.6 | NP_744688.1 |
| 69.7 | WP_072932993.1 | 58 | WP_043862968.1 | 52.5 | WP_057232912.1 |
| 66.9 | WP_038411221.1 | 60.8 | WP_048380564.1 | 56.2 | WP_045304617.1 |
| 67.5 | WP_011058720.1 | 57.7 | WP_064121969.1 | 54.1 | WP_029543520.1 |
| 68.3 | WP_037016311.1 | 58.6 | WP_025423867.1 | 53 | WP_067252507.1 |
| 68.5 | NP_742451.1 | 59.6 | WP_038410982.1 | 52.5 | WP_073739016.1 |
| 66.4 | WP_043255922.1 | 58.4 | WP_025246513.1 | 52.5 | WP_050373630.1 |
| 66.7 | WP_014335790.1 | 60.3 | WP_005175849.1 | 54.7 | WP_045694310.1 |
| 67 | WP_013696499.1 | 59.5 | WP_014361853.1 | 53 | WP_007264072.1 |
| 66.4 | WP_044412957.1 | 58.7 | NP_746862.1 | 54.9 | XP_007472004.1 |
| 66.3 | WP_058069294.1 | 60.2 | WP_076475776.1 | 52.6 | WP_053743099.1 |
| 65.1 | WP_004901085.1 | 58.8 | XP_007460921.1 | 51.1 | WP_013003566.1 |
| 67.9 | WP_025901143.1 | 57.6 | WP_045483576.1 | 54.3 | WP_046259890.1 |
| 65.8 | WP_023272348.1 | 57.1 | WP_009405810.1 | 51.4 | WP_009340495.1 |
| 63.3 | WP_004902770.1 | 59.6 | WP_070353966.1 | 53.4 | WP_009401519.1 |
| 66.7 | WP_043191662.1 | 55.1 | WP_067559336.1 | 51.7 | WP_051878118.1 |
| 66.9 | NP_248819.1 | 59.3 | WP_056546982.1 | 55.8 | WP_047312265.1 |
| 66.2 | WP_067430523.1 | 57.8 | WP_023955591.1 | 50.9 | WP_058944236.1 |
| 66.7 | WP_038023306.1 | 60.3 | WP_056917494.1 | 51.1 | WP_004956297.1 |
| 66.1 | WP_014085530.1 | 57.7 | WP_067018690.1 | 51.9 | WP_053727394.1 |
| 66.5 | WP_004549937.1 | 58.9 | WP_052590976.1 | 51.5 | WP_053659247.1 |
| 66.5 | WP_059571691.1 | 54 | WP_067555360.1 | 52.2 | WP_057583217.1 |
| 65.4 | WP_045847475.1 | 52.6 | WP_016655379.1 | 53.9 | WP_018778527.1 |
| 54.4 | WP_046586234.1 | 51.9 | WP_070027124.1 | 51.1 | WP_056558885.1 |
| 53.1 | WP_061920001.1 | 50.8 | WP_010986828.1 | 50.6 | WP_039649579.1 |
| 51 | WP_031129244.1 | 52.3 | WP_058850996.1 | 51.9 | WP_044381552.1 |
| 51.6 | WP_014045938.1 | 53.4 | WP_073491576.1 | 51.3 | WP_009949812.1 |
| 55.1 | WP_038413955.1 | 50.8 | WP_030229618.1 | 50.1 | WP_014672725.1 |
| 54.2 | WP_013792849.1 | 50.6 | WP_071367409.1 | 53.5 | WP_004902766.1 |
| 52 | WP_031040533.1 | 53 | WP_011693954.1 | 52.2 | WP_066501645.1 |
| 52.2 | WP_067556889.1 | 52.3 | WP_016435168.1 | 52.1 | WP_015863634.1 |
| 51.7 | WP_067134973.1 | 51.3 | WP_067159025.1 | 50.3 | WP_006137328.1 |
| 50.4 | WP_048479364.1 | 50.8 | WP_054214930.1 | 50.1 | WP_067213580.1 |
| 50.8 | WP_069920287.1 | 53.6 | WP_073938502.1 | 53.3 | WP_053732282.1 |
| 53.2 | WP_045066331.1 | 51.7 | WP_057574650.1 | 51.2 | WP_051523636.1 |
| 52.5 | WP_053747507.1 | 50.7 | WP_053741825.1 | 50.7 | WP_000360123.1 |
| 52.1 | WP_077006021.1 | 51.8 | WP_067370191.1 | 50.7 | NP_833449.1 |
| 52.8 | WP_013602901.1 | 51.1 | WP_069928754.1 | 52.5 | WP_004964863.1 |
| 51.6 | WP_015033615.1 | 50.8 | WP_073893967.1 | 52 | WP_000190767.1 |
| 50.7 | WP_053674902.1 | 51 | WP_053761118.1 | | |
| 50.9 | WP_073751185.1 | 51.9 | WP_067039494.1 | | |
| 51.7 | WP_052855765.1 | 51.2 | WP_067314493.1 | | |
| 51.2 | WP_073915189.1 | 50.7 | WP_069567714.1 | | |
| 51.9 | WP_009717917.1 | 51.1 | WP_045316288.1 | | |
| 51.8 | WP_023419456.1 | 51.1 | WP_065959379.1 | | |
| 51.4 | WP 030352392.1 | 51 | WP_023550727.1 | | |
| 52.1 | WP_014179663.1 | 51.1 | WP_053645448.1 | | |
| 52.5 | WP_003800151.1 | 52.7 | WP_067432696.1 | | |
| 54.7 | WP_030279738.1 | 51.5 | WP_005267979.1 | | |
| 51.3 | WP_005317561.1 | 50.8 | WP_054244935.1 | | |
| 51.2 | WP_053710239.1 | 50.4 | WP_061923084.1 | | |
| 51.7 | WP_031182291.1 | 52.6 | WP_062669880.1 | | |
| 50.8 | WP_030036242.1 | 51.9 | WP_069569483.1 | | |
| 52.1 | WP_067273400.1 | 52.8 | WP_053751366.1 | | |
| 53.4 | WP_053748975.1 | 52.8 | WP_030818466.1 | | |
| 52.3 | WP_069950078.1 | 53.6 | WP_073931875.1 | | |
| 51.4 | WP_062144595.1 | 51.7 | WP_049658990.1 | | |
| 52.8 | WP_016641286.1 | 52.9 | WP_031130834.1 | | |
| 53.6 | WP_070380447.1 | 53.4 | WP_053130852.1 | | |
| 50.2 | WP_058925293.1 | 52.9 | WP_043500873.1 | | |
| 54.1 | WP 012689404.1 | 50.3 | WP_037935270.1 | | |
| 50.9 | WP_020274291.1 | 50.3 | WP_046428867.1 | | |
| 51.1 | WP_062773918.1 | 50.3 | WP_067003524.1 | | |
| 50.9 | WP_003960477.1 | 53.1 | WP_015660379.1 | | |
| 52.3 | WP_056429296.1 | 50.4 | WP_014142508.1 | | |

Representative homologs for csiR include, for example, those in Table 5 below:

TABLE 5
csiR Homologs

| identity | accession | identity | accession | identity | accession |
|---|---|---|---|---|---|
| 99.1 | NP_708477.2 | 79.6 | WP_025801405.1 | 57.1 | WP_051496561.1 |
| 89.8 | WP_012134775.1 | 72.9 | WP_076942562.1 | 56.3 | WP_021817631.1 |
| 89.4 | WP_047461758.1 | 73.1 | WP_024910326.1 | 59 | WP_072820777.1 |
| 88.4 | WP_042288480.1 | 61.8 | WP_016415230.1 | 54.5 | WP_009096767.1 |
| 88 | NP_461720.1 | 59 | WP_071946013.1 | 54.9 | WP_007111526.1 |
| 88 | WP_000126152.1 | 58.9 | WP_045993733.1 | 56.1 | WP_071230235.1 |
| 88 | WP_000126159.1 | 60.8 | WP_035593369.1 | 54 | WP_070057168.1 |
| 88.3 | WP_043081738.1 | 60.3 | WP_029866289.1 | 56.5 | WP_035471761.1 |
| 86.1 | WP_017879787.1 | 61.7 | WP_043253788.1 | 55.5 | WP_071473537.1 |
| 86.1 | WP_002889794.1 | 58.7 | WP_075368656.1 | 55.7 | WP_014291195.1 |
| 83.4 | WP_013364902.1 | 59.4 | WP_013332139.1 | 51.7 | WP_076460165.1 |
| 84 | WP_002442796.1 | 59.3 | NP_745052.1 | | |
| 84.2 | WP_038163099.1 | 57.7 | WP_076748073.1 | | |

Representative homologs for gdhA include, for example, those in Table 6 below:

TABLE 6
gdhA Homologs

| identity | accession | identity | accession | identity | accession |
|---|---|---|---|---|---|
| 99.3 | NP_310494.1 | 83 | WP_004238567.1 | 71.3 | WP_023263455.1 |
| 99.3 | WP_000373050.1 | 82.3 | WP_038237533.1 | 71.5 | WP_014206092.1 |
| 93.5 | WP_061284831.1 | 81.7 | WP_012986712.1 | 71.3 | WP_065995578.1 |
| 93.1 | WP_012132655.1 | 81.4 | WP_024910597.1 | 71.7 | WP_005027261.1 |
| 93.1 | WP_012132655.1 | 81.4 | WP_076943162.1 | 72.4 | NP_879240.1 |
| 92.8 | NP_460265.1 | 82.6 | WP_005290714.1 | 71 | WP_010863788.1 |
| 92.8 | NP_456213.1 | 79.2 | WP_027273578.1 | 71.8 | WP_043379368.1 |
| 92.2 | WP_000372870.1 | 79 | WP_047779575.1 | 71.8 | WP_054065520.1 |
| 91.9 | WP_060569643.1 | 78.5 | WP_029093940.1 | 71.1 | WP_036246576.1 |
| 91.7 | WP_012905568.1 | 77.6 | WP_012697356.1 | 71.6 | WP_004263677.1 |
| 91.1 | WP_042392967.1 | 78.7 | WP_026823237.1 | 71.5 | WP_004912492.1 |
| 90.2 | WP_004176526.1 | 76.1 | WP_066610767.1 | 70.7 | WP_066150724.1 |
| 89.9 | WP_002901393.1 | 75.8 | WP_005763100.1 | 70.4 | WP_069040256.1 |
| 90.6 | WP_013366584.1 | 76.2 | WP_011979027.1 | 70.7 | WP_044401405.1 |
| 89.7 | WP_038154390.1 | 74.8 | WP_005724447.1 | 70.6 | WP_067559157.1 |
| 89.3 | WP_047369939.1 | 74.1 | WP_012341150.1 | 70.7 | WP_075147569.1 |
| 88.4 | WP_048278221.1 | 74.2 | WP_005761260.1 | 70.9 | WP_004342699.1 |
| 88.8 | WP_023481759.1 | 74.7 | WP_006717034.1 | 70.2 | WP_075585213.1 |
| 88.4 | WP_034493806.1 | 72.7 | WP_011869951.1 | 70.7 | WP_021248196.1 |
| 87.9 | WP_016535221.1 | 74.1 | NP_438358.1 | 70.7 | WP_069106631.1 |
| 88.1 | WP_002433734.1 | 72.5 | WP_034617651.1 | 70 | WP_011238883.1 |
| 88.1 | WP_002440315.1 | 74.4 | WP_012418820.1 | 69.8 | WP_066127634.1 |
| 87 | WP_025799484.1 | 72.2 | WP_061497951.1 | 69.5 | WP_010812813.1 |
| 85.5 | WP_024558048.1 | 73.5 | WP_029577560.1 | 70 | WP_047872672.1 |
| 85.8 | WP_068438947.1 | 72 | WP_039394299.1 | 69.8 | WP_020226927.1 |
| 85 | WP_011091696.1 | 72.2 | WP_013742507.1 | 71.8 | WP_011705519.1 |
| 98.5 | NP_707352.1 | 73.3 | WP_063491616.1 | 70.2 | WP_012153793.1 |
| 83.4 | WP_061799573.1 | 72.5 | WP_050417570.1 | 70.4 | WP_045846936.1 |
| 83.9 | WP_066749560.1 | 71.6 | WP_035524175.1 | 70.2 | WP_057291122.1 |
| 84.8 | WP_011144528.1 | 71.6 | WP_056665786.1 | 70.9 | WP_012206939.1 |
| 83.9 | WP_002209529.1 | 72 | WP_005881531.1 | 69.3 | WP_009523677.1 |
| 84.8 | WP_021323407.1 | 71.8 | WP_055422989.1 | 69.1 | WP_011804418.1 |
| 83.4 | WP_053907316.1 | 71.1 | WP_066885513.1 | 69.8 | WP_022771702.1 |
| 83 | WP_004722623.1 | 72.2 | WP_046682209.1 | 71 | XP_001470338.1 |
| 83.9 | WP_056235126.1 | 70.3 | WP_073101398.1 | 70.2 | WP_055892111.1 |
| 82.6 | WP_010848400.1 | 71.8 | WP_076095136.1 | 69.5 | WP_054254790.1 |
| 83.2 | WP_034790893.1 | 72.5 | WP_004265379.1 | 70.2 | WP_057666947.1 |
| 84.2 | WP_008914299.1 | 72.3 | WP_066536423.1 | 68.8 | WP_071022768.1 |
| 82.6 | WP_045967805.1 | 72.2 | WP_002924997.1 | 69.5 | WP_013899919.1 |
| 81.7 | WP_037385419.1 | 71.7 | WP_002017215.1 | 69.5 | WP_066084376.1 |
| 69.7 | WP_070068869.1 | 70.9 | XP_809575.1 | 63.7 | WP_072556406.1 |
| 70.5 | WP_009516161.1 | 66.7 | WP_066077554.1 | 63.5 | WP_006989144.1 |
| 68.4 | WP_011381737.1 | 70.4 | XP_818855.1 | 63.3 | WP_014782294.1 |
| 67.6 | WP_012018270.1 | 67 | NP_274713.1 | 64.6 | WP_045970664.1 |
| 68.1 | WP_014238223.1 | 65.5 | WP_027589584.1 | 63.1 | WP_015024364.1 |
| 68.8 | WP_005673352.1 | 66.8 | WP_003691714.1 | 64.3 | WP_047760385.1 |
| 67.7 | WP_046849612.1 | 65 | WP_038411996.1 | 63.3 | WP_041518578.1 |
| 69.5 | WP_056899245.1 | 64.8 | XP_007470804.1 | 62.3 | WP_009778489.1 |
| 69.7 | WP_023273806.1 | 64.1 | WP_037025837.1 | 65.5 | WP_023572750.1 |
| 69.9 | WP_057632954.1 | 66.7 | WP_041961412.1 | 63.2 | WP_013869486.1 |
| 68.2 | WP_013967258.1 | 64.1 | WP_068711253.1 | 63.9 | WP_025217351.1 |
| 68.4 | WP_011112180.1 | 65.1 | WP_013750796.1 | 64 | WP_047788213.1 |
| 70.3 | XP_001684577.1 | 66.3 | WP_003787270.1 | 63.2 | WP_038532563.1 |
| 68.8 | XP_001566338.1 | 66.4 | WP_003794518.1 | 62.2 | WP_072553580.1 |
| 67.7 | WP_004180599.1 | 65.2 | WP_007358976.1 | 63.3 | WP_035636319.1 |
| 68.4 | WP_070528401.1 | 65.9 | WP_005600521.1 | 63.9 | WP_067593854.1 |
| 72.6 | XP_007459563.1 | 66 | WP_004581059.1 | 63.4 | WP_072877073.1 |
| 66.8 | WP_074796977.1 | 64.2 | WP_064502110.1 | 63.7 | WP_066333123.1 |
| 68.6 | WP_013646221.1 | 65.3 | WP_054408663.1 | 63.7 | WP_068702329.1 |
| 68.4 | WP_073355061.1 | 63.8 | WP_045483724.1 | 62.8 | WP_072316830.1 |
| 67.7 | WP_068367791.1 | 66.4 | WP_014389636.1 | 63.5 | WP_035127618.1 |
| 69.2 | WP_013534127.1 | 64.9 | WP_068590903.1 | 62.1 | XP_014150269.1 |
| 66.2 | WP_068388615.1 | 65 | WP_007525492.1 | 62.3 | WP_008255576.1 |
| 65.8 | WP_048378258.1 | 65.5 | WP_072782102.1 | 62 | WP_045445530.1 |
| 68.6 | WP_034292074.1 | 66.2 | WP_014166676.1 | 61.4 | WP_007295137.1 |
| 66.7 | WP_072293362.1 | 64.2 | WP_035133178.1 | 61.9 | WP_073149023.1 |
| 67.7 | WP_011479508.1 | 63.6 | WP_053100289.1 | 61.2 | WP_007291459.1 |
| 70.9 | NP_295441.1 | 64.8 | WP_058885994.1 | 63.1 | WP_073192467.1 |
| 67 | WP_072767836.1 | 64.8 | WP_004140087.1 | 63.4 | WP_007646985.1 |
| 66.6 | WP_058068191.1 | 64.5 | WP_066106171.1 | 63 | WP_008270126.1 |
| 65.1 | NP_253278.1 | 64.4 | WP_011962445.1 | 61.9 | WP_013186270.1 |
| 65.4 | WP_011063568.1 | 63.7 | WP_014085198.1 | 62.5 | WP_071670940.1 |
| 67 | WP_069320101.1 | 66 | WP_073361754.1 | 62.9 | WP_066432262.1 |
| 66.1 | WP_013261011.1 | 64.9 | WP_004283594.1 | 61.8 | WP_014202483.1 |
| 68.1 | WP_057640843.1 | 64.9 | WP_036990740.1 | 60.7 | WP_057951377.1 |
| 66.7 | WP_021701106.1 | 63.4 | WP_008236677.1 | 61.3 | WP_071184514.1 |
| 65.8 | WP_043860208.1 | 64.7 | WP_009117794.1 | 62 | WP_014032453.1 |
| 67.7 | WP_046741930.1 | 62.7 | WP_013792808.1 | 59.5 | WP_062080697.1 |
| 67.2 | WP_009425920.1 | 64 | WP_056066801.1 | 62.8 | WP_076619600.1 |
| 65.3 | WP_009398976.1 | 64.2 | WP_064716051.1 | | |
| 65.7 | NP_742836.1 | 62.1 | WP_066311622.1 | | |
| 65.9 | WP_028461562.1 | 64.1 | WP_015432444.1 | | |

Representative homologs for rcsA include, for example, those in Table 7 below:

TABLE 7
rcsA Homologs

| identity | accession | identity | accession | identity | accession |
|---|---|---|---|---|---|
| 99.5 | NP_707836.1 | 81.2 | WP_034496363.1 | 67.6 | WP_002911561.1 |
| 99 | WP_000103987.1 | 83.6 | WP_041851838.1 | 66 | WP_076941361.1 |
| 92.8 | WP_012131895.1 | 81.6 | WP_013365795.1 | 67 | WP_040173869.1 |
| 89.9 | WP_042284059.1 | 82.1 | WP_016537069.1 | 64 | WP_006119584.1 |
| 88.4 | WP_012906243.1 | 82.6 | WP_024558645.1 | 66.5 | WP_024911967.1 |
| 88.4 | WP_000103971.1 | 82.1 | WP_042391443.1 | 65.6 | WP_004961281.1 |
| 87.9 | NP_460935.1 | 79.7 | WP_036102225.1 | 64 | WP_013202686.1 |
| 87.4 | NP_456543.1 | 79.2 | WP_061279235.1 | 64 | WP_016189509.1 |
| 86.5 | WP_000103977.1 | 77.8 | WP_002434933.1 | 63.5 | WP_034935559.1 |
| 86.5 | WP_000103977.1 | 72.5 | WP_043083214.1 | 63.5 | WP_052900395.1 |
| 83.6 | WP_023479186.1 | 67.5 | WP_061797360.1 | 62.4 | WP_025421842.1 |
| 83.6 | WP_038153230.1 | 67.6 | WP_002911561.1 | 61.1 | WP_012441725.1 |
| | | | | 60.7 | WP_056236553.1 |

Representative homologs for cspB include, for example, those in Table 8 below:

TABLE 8
cspB Homologs

| identity | accession | identity | accession | identity | accession |
|---|---|---|---|---|---|
| 99.2 | NP_310881.1 | 64.5 | WP_068371324.1 | 61.3 | WP_011190167.1 |
| 99.2 | NP_707944.1 | 62.6 | WP_036542817.1 | 80.4 | WP_016537210.1 |
| 95.4 | WP_012131712.1 | 63.9 | WP_046007005.1 | 61.8 | WP_070175995.1 |
| 95.4 | WP_012131712.1 | 62.4 | WP_056233316.1 | 61 | WP_002227829.1 |
| 94.6 | WP_060569271.1 | 63.8 | WP_044617625.1 | 62.5 | WP_005368980.1 |
| 93.5 | WP_012906388.1 | 61.7 | WP_002441831.1 | 60 | WP_013026371.1 |
| 91.8 | WP_023479222.1 | 63.8 | WP_007103720.1 | 62.6 | WP_008246700.1 |
| 91.2 | WP_038162876.1 | 63.7 | WP_011149325.1 | 60.6 | WP_075609504.1 |
| 90.8 | NP_461050.3 | 61.8 | WP_020584511.1 | 60.3 | WP_013319591.1 |
| 88.9 | WP_047369611.1 | 61.4 | WP_004180506.1 | 60.8 | WP_011045707.1 |
| 89.1 | WP_013365645.1 | 62.1 | WP_067083187.1 | 61.8 | WP_042502510.1 |
| 88.3 | WP_042389857.1 | 62.7 | WP_007617499.1 | 60.2 | WP_043383353.1 |
| 89 | WP_024555780.1 | 63.5 | WP_016402533.1 | 59.3 | NP_461029.1 |
| 84.4 | WP_034496621.1 | 63.2 | WP_033078671.1 | 59.8 | WP_004074522.1 |
| 83.6 | WP_036115077.1 | 61.2 | WP_004144154.1 | 59.4 | WP_046218786.1 |
| 80.6 | WP_002434790.1 | 60.5 | WP_026742150.1 | 60.5 | WP_016414892.1 |
| 77 | WP_034789901.1 | 61 | WP_002434803.1 | 61 | WP_013164038.1 |
| 73.3 | WP_072928432.1 | 61.9 | WP_010361457.1 | 60.2 | WP_011769177.1 |
| 66.5 | WP_021016755.1 | 61.9 | WP_066852904.1 | 59.4 | WP_000040079.1 |
| 67.5 | WP_034774893.1 | 64.4 | WP_047010547.1 | 62.1 | WP_014869963.1 |
| 67.8 | WP_008983873.1 | 62.5 | WP_007635207.1 | 60.3 | WP_070979447.1 |
| 66.7 | WP_076575252.1 | 62.8 | WP_054340040.1 | 61.6 | WP_013338389.1 |
| 65.1 | WP_043115298.1 | 60.7 | WP_008871074.1 | 59.5 | WP_048278099.1 |
| 64.2 | WP_027706165.1 | 62.4 | WP_048693221.1 | 61.5 | WP_008595267.1 |
| 84.9 | WP_016537234.1 | 60.7 | WP_070124087.1 | 59.2 | WP_062477440.1 |
| 64.7 | WP_015879117.1 | 62.6 | WP_045994725.1 | 59.2 | WP_070069283.1 |
| 65.3 | WP_015045926.1 | 64.5 | WP_064124575.1 | 58.3 | WP_038018172.1 |
| 64.4 | WP_008900819.1 | 62.3 | WP_061798502.1 | 61 | WP_014109020.1 |
| 63.2 | WP_013050748.1 | 61.3 | WP_068065091.1 | 60.9 | WP_011093022.1 |
| 63.3 | WP_044058200.1 | 61.4 | WP_073322754.1 | 61.4 | WP_067151100.1 |
| 60.5 | NP_310863.1 | 63.7 | WP_074780011.1 | 61.4 | NP_636017.1 |
| 64.8 | WP_067085093.1 | 62.9 | WP_071945886.1 | 61.5 | WP_009451320.1 |
| 64.3 | WP_036185668.1 | 61.7 | WP_013053243.1 | 60.9 | WP_005506467.1 |
| 63.6 | WP_008844102.1 | 59.8 | WP_067432072.1 | 61.6 | WP_012917163.1 |
| 62.2 | WP_055023157.1 | 62.5 | WP_014871466.1 | 60.2 | WP_015961114.1 |
| 63.8 | WP_037443797.1 | 62.2 | WP_035015963.1 | 59.2 | WP_043099967.1 |
| 62.2 | WP_025421658.1 | 62 | WP_000938488.1 | 56.2 | WP_072287528.1 |
| 62 | WP_025245289.1 | 60.2 | WP_021185375.1 | 58.4 | WP_046860934.1 |
| 60.5 | WP_013790788.1 | 59.9 | WP_008898411.1 | 60.8 | WP_012401674.1 |
| 59.2 | WP_072909217.1 | 59.1 | WP_035516982.1 | 55.5 | WP_015487669.1 |
| 59.5 | WP_046009100.1 | 59.5 | WP_057666743.1 | 54.7 | WP_015832722.1 |
| 58.2 | WP_072282418.1 | 58.4 | WP_074497678.1 | 56.7 | WP_038495097.1 |
| 58.8 | WP_058642714.1 | 57.7 | WP_029577397.1 | 56.5 | WP_008446417.1 |
| 58.5 | WP_074727698.1 | 58.4 | WP_036115097.1 | 58.1 | WP_050408890.1 |
| 59.7 | WP_055936344.1 | 55.9 | WP_012979494.1 | 56.8 | WP_067378655.1 |
| 59.3 | WP_066106278.1 | 57.2 | WP_056131859.1 | 56.5 | WP_012418277.1 |
| 58.5 | WP_053551060.1 | 56.2 | WP_014293403.1 | 57.1 | WP_012506278.1 |
| 57.1 | WP_016416607.1 | 58.6 | WP_047823239.1 | 55.7 | WP_069038181.1 |
| 57.2 | WP_013163635.1 | 56.6 | WP_023659974.1 | 56.7 | WP_011387757.1 |
| 56.8 | WP_014149193.1 | 55 | WP_009725082.1 | 56 | WP_067268066.1 |
| 60.5 | WP_076720076.1 | 59.5 | WP_057508396.1 | 58.5 | WP_072754637.1 |
| 58.6 | WP_008220422.1 | 55.1 | WP_066980484.1 | 53.9 | WP_007876327.1 |
| 59.4 | WP_057658318.1 | 57.8 | WP_011396291.1 | 54.8 | WP_015005922.1 |
| 57.4 | WP_036272759.1 | 57.3 | WP_009669786.1 | 56 | WP_015773966.1 |
| 57.7 | WP_026816601.1 | 58.9 | WP_057646291.1 | 55.8 | WP_011361564.1 |
| 58.9 | WP_022969261.1 | 57.5 | WP_048670591.1 | 56.7 | WP_004581651.1 |
| 58.9 | WP_052631775.1 | 58.2 | WP_047251055.1 | 57 | WP_066124619.1 |
| 56.7 | WP_066919190.1 | 55.8 | WP_006000332.1 | 56.6 | WP_057158792.1 |
| 58.4 | WP_071473820.1 | 58.2 | WP_056339205.1 | 56.6 | WP_014404181.1 |
| 58.1 | WP_070049952.1 | 55.8 | WP_045476806.1 | 56.4 | WP_068803525.1 |
| 56.9 | WP_040101190.1 | 56.9 | WP_007232600.1 | 55 | WP_068867762.1 |
| 59.3 | WP_014159972.1 | 58 | WP_036237504.1 | 56.1 | WP_037451266.1 |
| 59.1 | WP_052632400.1 | 56.9 | WP_066884247.1 | 55 | WP_011127024.1 |
| 57.7 | WP_040201620.1 | 58.1 | WP_070057788.1 | 54.3 | WP_007042797.1 |
| 57.8 | WP_075998792.1 | 56.9 | WP_070985717.1 | 57.3 | WP_057654273.1 |
| 57.2 | WP_066055642.1 | 58.2 | WP_005669198.1 | 55.5 | WP_013163951.1 |
| 57.2 | WP_046552797.1 | 55.5 | WP_037386168.1 | 56 | WP_011935067.1 |
| 60.4 | WP_057641601.1 | 57.1 | WP_008118279.1 | 55.6 | WP_067012826.1 |
| 57.6 | WP_007146565.1 | 54.9 | WP_006786367.1 | 54.4 | WP_034222136.1 |
| 55.9 | WP_054758791.1 | 55.8 | WP_069297126.1 | 55.2 | WP_016493684.1 |
| 59.7 | WP_013536105.1 | 58.3 | WP_073217888.1 | 57.1 | WP_045492964.1 |
| 57.5 | WP_011378111.1 | 57.5 | WP_015830447.1 | 56.5 | WP_013795032.1 |
| 57.9 | WP_059756905.1 | 57.4 | WP_038414439.1 | 55.6 | WP_012018200.1 |
| 57.4 | WP_013817915.1 | 58.6 | WP_013233989.1 | 55.4 | WP_038639575.1 |
| 58.1 | WP_011311751.1 | 56.8 | NP_661213.1 | 55.8 | WP_006909293.1 |
| 57.3 | WP_023496011.1 | 58.2 | WP_036252703.1 | 54.9 | WP_015904322.1 |
| 58.6 | WP_014200351.1 | 55.9 | WP_004965212.1 | 55.6 | WP_053938685.1 |
| 57.1 | WP_002691865.1 | 56.9 | WP_012466687.1 | 57.7 | WP_034872762.1 |
| 58.4 | WP_057631722.1 | 56.9 | WP_036164401.1 | 56.1 | WP_007277330.1 |
| 59 | WP_033534182.1 | 57.4 | WP_055831483.1 | 55.9 | WP_043949824.1 |
| 55.7 | WP_005224278.1 | 57.8 | WP_054267015.1 | 55.7 | WP_004251173.1 |
| 55.7 | WP_007080267.1 | 54.4 | WP_008914131.1 | 55.3 | WP_069520026.1 |
| 56.4 | WP_044432231.1 | | | | |

Representative homologs for cspG include, for example, those in Table 9 below:

TABLE 9
cspG Homologs

| identity | accession | identity | accession | identity | accession |
|---|---|---|---|---|---|
| 99.1 | NP_310880.1 | 71.2 | WP_013026370.1 | 65.1 | WP_011978707.1 |
| 99.1 | NP_707943.2 | 69.9 | WP_045111744.1 | 65.6 | WP_034872763.1 |
| 97.4 | NP_310862.1 | 69.6 | WP_067384104.1 | 66.8 | WP_021014453.1 |
| 92.5 | WP_012131713.1 | 70.9 | WP_053396882.1 | 66.3 | WP_067086935.1 |
| 92.1 | WP_012906387.1 | 69.4 | WP_013319592.1 | 66.6 | WP_053551059.1 |
| 92.4 | WP_060569270.1 | 69.7 | WP_012737914.1 | 63.4 | WP_015817378.1 |
| 89.5 | NP_461049.1 | 70.4 | WP_021185376.1 | 62.6 | WP_007617502.1 |
| 89.5 | WP_001038235.1 | 70 | WP_066106815.1 | 62.4 | WP_008844103.1 |
| 88.8 | WP_044782506.1 | 69.8 | WP_068065094.1 | 65.2 | WP_002730774.1 |
| 88.2 | WP_038162879.1 | 69.4 | WP_076575253.1 | 64.8 | WP_040201621.1 |
| 88.4 | WP_048888096.1 | 69.3 | WP_033078672.1 | 62.2 | WP_007635206.1 |
| 86.8 | WP_036115080.1 | 67.9 | WP_007232649.1 | 62.8 | WP_070124086.1 |
| 87.4 | WP_000164173.1 | 68.2 | WP_005763626.1 | 64.8 | WP_011341527.1 |
| 86.6 | WP_023479224.1 | 70.1 | WP_012487719.1 | 64.3 | WP_046218785.1 |
| 85.5 | WP_047373219.1 | 68.2 | WP_012340250.1 | 65.4 | WP_067151117.1 |
| 83.5 | WP_042389858.1 | 69.2 | WP_072429743.1 | 63.3 | WP_008248250.1 |
| 77.4 | WP_034789903.1 | 67.5 | WP_038018174.1 | 64.8 | WP_072287530.1 |
| 77.3 | WP_015879116.1 | 68.9 | WP_006719621.1 | 64 | WP_011341516.1 |
| 77.2 | WP_048278098.1 | 66.7 | WP_034832575.1 | 66.4 | WP_076878398.1 |
| 76.5 | WP_004899416.1 | 68.2 | WP_046098754.1 | 61.8 | WP_070175996.1 |
| 76.3 | WP_032248255.1 | 67.9 | WP_053938686.1 | 64.5 | WP_065994504.1 |
| 77.7 | WP_002441829.1 | 68.2 | WP_017805541.1 | 64.7 | WP_014208062.1 |
| 73.9 | WP_002208588.1 | 68.9 | WP_011199528.1 | 63.2 | WP_046009540.1 |
| 76 | WP_026742151.1 | 67.3 | NP_229899.1 | 64.9 | XP_003087927.1 |
| 74.3 | WP_021016754.1 | 66.8 | WP_058026143.1 | 62.8 | WP_006872614.1 |
| 75.3 | WP_056233314.1 | 67 | WP_074780041.1 | 62 | WP_011467483.1 |
| 75 | WP_067432069.1 | 67.1 | WP_025217396.1 | 63.9 | WP_058020695.1 |
| 74.1 | WP_026743145.1 | 67.5 | WP_015878502.1 | 61.2 | WP_046561424.1 |
| 75.2 | WP_023492173.1 | 68.1 | WP_066852902.1 | 62.7 | WP_014703321.1 |
| 73.8 | WP_061798540.1 | 67.5 | WP_034614782.1 | 62.5 | WP_047011196.1 |
| 74.4 | WP_014149652.1 | 64.3 | WP_007103721.1 | 60 | WP_075609503.1 |
| 73.5 | WP_034773678.1 | 67.4 | WP_013261133.1 | 64.2 | WP_000209967.1 |
| 71.7 | WP_011093023.1 | 66.4 | WP_011041175.1 | 60.4 | WP_041067437.1 |
| 75.2 | WP_002434805.1 | 66 | WP_005761892.1 | 62.4 | WP_072909216.1 |
| 73.6 | WP_025245291.1 | 65.7 | WP_074498014.1 | 63.3 | WP_006001260.1 |
| 73.4 | WP_025421659.1 | 67.1 | WP_011848443.1 | 63.9 | WP_023274036.1 |
| 72.1 | WP_046350411.1 | 66.7 | WP_015045899.1 | 64.9 | WP_004902846.1 |
| 70.5 | WP_035015962.1 | 63.5 | WP_068371327.1 | 59.9 | WP_044057049.1 |
| 70.4 | WP_020581561.1 | 65.4 | WP_007226624.1 | 63 | WP_044834099.1 |
| 70.2 | WP_036115099.1 | 65.4 | WP_044618080.1 | 63.2 | WP_070116464.1 |
| 62.8 | WP_016657988.1 | 59.1 | WP_007288147.1 | 54.8 | WP_037204061.1 |
| 62.7 | WP_007019331.1 | 58.5 | WP_004098872.1 | 57 | WP_014159340.1 |
| 63.3 | WP_011706704.1 | 59.5 | NP_743933.1 | 56.4 | WP_054966882.1 |
| 60.6 | WP_011879416.1 | 58.6 | WP_037011897.1 | 57.3 | WP_011338528.1 |
| 60.7 | WP_008292573.1 | 60.1 | WP_068614515.1 | 59.1 | WP_076533872.1 |
| 64.9 | WP_072899073.1 | 59.6 | WP_057508397.1 | 58.5 | WP_041044208.1 |
| 60.4 | WP_058506913.1 | 57.4 | WP_066919192.1 | 54.6 | WP_048600028.1 |
| 61.7 | WP_008043193.1 | 59.5 | WP_014869787.1 | 56.3 | WP_012939274.1 |
| 62.3 | WP_008220419.1 | 56.2 | WP_027890387.1 | 55.6 | WP_036366943.1 |
| 61.7 | WP_066055636.1 | 59.1 | WP_008216258.1 | 55.3 | WP_037281415.1 |
| 60.8 | WP_075998790.1 | 57.5 | WP_047251054.1 | 56.4 | WP_006914398.1 |
| 63.6 | WP_067551219.1 | 59.8 | WP_057666745.1 | 56 | WP_022572924.1 |
| 62.2 | WP_005000244.1 | 59.5 | WP_038414435.1 | 54.1 | WP_021771267.1 |
| 59.7 | WP_014109019.1 | 58.6 | WP_040882268.1 | 54.4 | WP_068356257.1 |
| 61.7 | WP_005017365.1 | 57.8 | WP_025281979.1 | 55.5 | WP_073615210.1 |
| 61.1 | WP_013163950.1 | 57.1 | WP_072934195.1 | 56.2 | WP_057658319.1 |
| 60.8 | WP_007230714.1 | 56.7 | WP_006190557.1 | 55 | WP_067337815.1 |
| 62.2 | WP_070068421.1 | 58.6 | WP_057641602.1 | 55.3 | WP_038650370.1 |
| 60.3 | WP_002210201.1 | 57.7 | WP_013536104.1 | 54.2 | WP_057757910.1 |
| 64.1 | WP_007187501.1 | 59.6 | WP_015279948.1 | 55.3 | WP_076756185.1 |
| 61.2 | WP_015756507.1 | 56.8 | WP_011371098.1 | 54.6 | WP_044831320.1 |
| 59.9 | WP_048670590.1 | 56.5 | WP_070049951.1 | 55.7 | WP_036717293.1 |
| 61 | WP_035516986.1 | 58.1 | WP_070057181.1 | 54.9 | WP_006693690.1 |
| 60.6 | WP_063674025.1 | 57.4 | WP_035629591.1 | 57.5 | WP_050518223.1 |
| 60.1 | WP_013012032.1 | 57.6 | WP_072306156.1 | 54.6 | WP_036738098.1 |
| 58.9 | WP_075757193.1 | 55.9 | WP_054258658.1 | 53.4 | WP_013108261.1 |
| 59.8 | WP_076941975.1 | 57.9 | WP_057631723.1 | 54.4 | WP_014559477.1 |
| 60.2 | WP_014257545.1 | 58.4 | WP_012917162.1 | 55.3 | WP_015404017.1 |
| 60.9 | WP_008316262.1 | 55.7 | WP_076555551.1 | 52 | WP_014856106.1 |
| 58.8 | WP_072321911.1 | 57.5 | WP_015256936.1 | 54.4 | WP_006460472.1 |
| 59.5 | WP_046552796.1 | 57.4 | WP_008030129.1 | 55.7 | WP_049726165.1 |
| 58.7 | WP_021169209.1 | 59.1 | WP_062214530.1 | 52.2 | WP_008034238.1 |
| 59.2 | WP_068090970.1 | 54.2 | WP_045967921.1 | 52.8 | WP_007279991.1 |
| 59.7 | WP_015774426.1 | 58.4 | WP_057646290.1 | 54.2 | WP_073372967.1 |
| 60 | WP_007233573.1 | 57 | WP_012281869.1 | 53.6 | WP_009016323.1 |
| 59.9 | WP_007930125.1 | 58.5 | WP_052632402.1 | 54.6 | WP_013163636.1 |
| 57.4 | WP_014809125.1 | 57.4 | WP_076449653.1 | 52 | WP_012600347.1 |
| 58.9 | WP_038673883.1 | 54.5 | WP_012823380.1 | | |
| 58.6 | WP_009470030.1 | 58.1 | WP_013168543.1 | | |
| 59.7 | WP_043189096.1 | 56.2 | WP_074969493.1 | | |
| 59.3 | WP_009020472.1 | 56.4 | WP_012638667.1 | | |
| 59.4 | WP_011400136.1 | 57.9 | NP_636018.1 | | |

If the desired product is 6-hydroxycaproic acid (6HCA) the production of this product can by engineering the production cell to overexpress yghD (Type II secretion system protein), yjgB (alcohol dehydrogenase), and/or yahK (aldehyde reductase).

Genetically modified cells (e. g. non-naturally occurring microorganisms) described herein can be capable of producing the nylon intermediates such as 6-aminocaproic acid, caprolactam, and hexamethylenediamine.

As used herein, the term “non-naturally occurring” when used in reference to a microbial organism or microorganism is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes within a 6-aminocaproic acid, caprolactam, hexamethylenediamine or levulinic acid biosynthetic pathway.

As used herein, the term “disruption” means to a native gene or promoter is mutated, deleted, interrupted, or down regulated in such a way as to decrease the activity of the gene and/or gene product in the host cell. A gene can be completely (100%) reduced by knockout or removal of the entire genomic DNA sequence. Use of a frame shift mutation, early stop codon, point mutations of critical residues, or deletions or insertions, and the like, can completely inactivate (100%) gene product by completely preventing transcription and/or translation of active protein.

A metabolic modification refers to a biochemical reaction that is altered from its naturally occurring state. Therefore, non-naturally occurring microorganisms can have genetic modifications to nucleic acids encoding metabolic polypeptides or, functional fragments thereof. Exemplary metabolic modifications are disclosed herein.

As used herein, the terms “microbial,” “microbial organism” or “microorganism” has been used interchangeably and is intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, “adipate,” having the chemical formula —OOC—(CH2)4-COO— (see FIG. 1) (IUPAC name hexanedioate), is the ionized form of adipic acid (IUPAC name hexanedioic acid), and it is understood that adipate and adipic acid can be used interchangeably throughout to refer to the compound in any of its neutral or ionized forms, including any salt forms thereof. It is understood by those skilled understand that the specific form will depend on the pH.

As used herein, “6-aminocaproate,” having the chemical formula —OOC—(CH2)5-NH2 (see FIG. 1, and abbreviated as 6-ACA), is the ionized form of 6-aminocaproic acid (IUPAC name 6-aminohexanoic acid), and it is understood that 6-aminocaproate and 6-aminocaproic acid can be used interchangeably throughout to refer to the compound in any of its neutral or ionized forms, including any salt forms thereof. It is understood by those skilled understand that the specific form will depend on the pH.

As used herein, “caprolactam” (IUPAC name azepan-2-one) is a lactam of 6-aminohexanoic acid (see FIG. 1, and abbreviated as CPO).

As used herein, “hexamethylenediamine,” also referred to as 1,6-diaminohexane or 1,6-hexanediamine, has the chemical formula H2N(CH2)6NH2 (see FIG. 1 and abbreviated as HMD).

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

As used herein, the term “growth-coupled” when used in reference to the production of a biochemical is intended to mean that the biosynthesis of the referenced biochemical is produced during the growth phase of a microorganism. In a particular embodiment, the growth-coupled production can be obligatory, meaning that the biosynthesis of the referenced biochemical is an obligatory product produced during the growth phase of a microorganism.

As used herein, “metabolic modification” is intended to refer to a biochemical reaction that is altered from its naturally occurring state. Metabolic modifications can include, for example, elimination of a biochemical reaction activity by functional disruptions of one or more genes encoding an enzyme participating in the reaction.

As used herein, the term “disruption,” “gene disruption,” or grammatical equivalents thereof, is intended to mean a genetic alteration that renders the encoded gene product inactive. The genetic alteration can be, for example, deletion of the entire gene, deletion of a regulatory sequence required for transcription or translation, deletion of a portion of the gene which results in a truncated gene product, or by any of various mutation strategies that inactivate the encoded gene product. One particularly useful method of gene disruption is complete gene deletion because it reduces or eliminates the occurrence of genetic reversions in the non-naturally occurring microorganisms.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism.

The term “heterologous” refers to a molecule, material, or activity derived from a source other than the referenced species whereas “homologous” refers to a molecule, material, or activity derived from the host microbial organism. Accordingly, exogenous expression of an encoding nucleic acid can utilize either or both a heterologous or homologous encoding nucleic acid.

It is understood that when more than one exogenous nucleic acid is included in a microbial organism, the exogenous nucleic acids refer to the referenced encoding nucleic acid or biosynthetic activity, as discussed above. It is further understood, as disclosed herein, that such exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, which are not integrated into the host chromosome, and the plasmids remain as extra-chromosomal elements, and still be considered as two or more exogenous nucleic acids. The number of referenced exogenous nucleic acids or biosynthetic activities refers to the number of encoding nucleic acids or the number of biosynthetic activities, not the number of separate nucleic acids introduced into the host organism.

The non-naturally occurring microbial organisms can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

In the case of gene disruptions, a particularly useful stable genetic alteration is a gene deletion. The use of a gene deletion to introduce a stable genetic alteration is particularly useful to reduce the likelihood of a reversion to a phenotype prior to the genetic alteration. For example, stable growth-coupled production of a biochemical can be achieved, for example, by deletion of a gene encoding an enzyme catalyzing one or more reactions within a set of metabolic modifications. The stability of growth-coupled production of a biochemical can be further enhanced through multiple deletions, significantly reducing the likelihood of multiple compensatory reversions occurring for each disrupted activity.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less than 25% can also be considered to have arisen by vertical descent if their three-dimensional structure also shows similarities. Members of the serine protease family of enzymes, including tissue plasminogen activator and elastransaminasee, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastransaminasee proteolysis and plasminogen proteolysis, two types of serine protease activity, into distinct molecules as plasminogen activator and elastransaminasee. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms having 6-aminocaproic acid, caprolactam, hexamethylenediamine or levulinic acid biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes. In gene disruption strategies, evolutionally related genes can also be disrupted or deleted in a host microbial organism, paralogs or orthologs, to reduce or eliminate activities to ensure that any functional redundancy in enzymatic activities targeted for disruption do not short circuit the designed metabolic modifications.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary paramemeters for determining relatedness of two or more sequences using the BLAST algorithm, for example, can be as set forth below. Briefly, amino acid sequence alignments can be performed using BLASTP version 2. 2. 29+(Jan. 14, 2014) and the following parameTransaminase: Matrix: 0 BLOSUM62; gap open: 11; gap extension: 1; x_dropoff: 50; expect: 10. 0; wordsize: 3; filter: on. Nucleic acid sequence alignments can be performed using BLASTN version 2. 0. 6 (Sep. 16, 1998) and the following parameTransaminase: Match: 1; mismatch: −2; gap open: 5; gap extension: 2; x_dropoff: 50; expect: 10. 0; wordsize: 11; filter: off. Those skilled in the art will know what modifications can be made to the above parameTransaminase to either increase or decrease the stringency of the comparison, for example, and determine the relatedness of two or more sequences.

It is understood that any of the pathways disclosed herein, including those as described in the Figures can be used to generate a non-naturally occurring microbial organism that produces any pathway intermediate or product, as desired. As disclosed herein, such a microbial organism that produces an intermediate can be used in combination with another microbial organism expressing downstream pathway enzymes to produce a desired product. However, it is understood that a non-naturally occurring microbial organism that produces a 6-aminocaproic acid, caprolactam, or hexamethylenediamine can be utilized to produce the intermediate as a desired product.

Described herein with general reference to the metabolic reaction, reactant or product thereof, or with specific reference to one or more nucleic acids or genes encoding an enzyme associated with or catalyzing the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze the referenced reaction, reactant or product. Likewise, given the well-known fields of metabolic biochemistry, enzymology and genomics, reference herein to a gene or encoding nucleic acid also constitutes a reference to the corresponding encoded enzyme and the reaction it catalyzes as well as the reactants and products of the reaction.

The non-naturally occurring microbial organisms can be produced by introducing expressible nucleic acids encoding one or more of the enzymes participating in one or more 6-aminocaproic acid, caprolactam, hexamethylenediamine, or other C5-C14 biosynthetic pathways. Depending on the host microbial organism chosen for biosynthesis, nucleic acids for some or all of a particular 6-aminocaproic acid, caprolactam, hexamethylenediamine, or other C5-C14 biosynthetic pathway can be expressed. For example, if a chosen host is deficient in one or more enzymes for a desired biosynthetic pathway, then expressible nucleic acids for the deficient enzyme(s) are introduced into the host for subsequent exogenous expression. Alternatively, if the chosen host exhibits endogenous expression of some pathway genes, but is deficient in others, then an encoding nucleic acid is needed for the deficient enzyme(s) to achieve 6-aminocaproic acid, caprolactam, hexamethylenediamine or other C5-C14 product biosynthesis. Thus, a non-naturally occurring microbial organism can be produced by introducing exogenous enzyme activities to obtain a desired biosynthetic pathway or a desired biosynthetic pathway can be obtained by introducing one or more exogenous enzyme activities that, together with one or more endogenous enzymes, produce a desired product such as 6-aminocaproic acid, caprolactam, or hexamethylenediamine.

Depending on the 6-aminocaproic acid, caprolactam, hexamethylenediamine or other C5-C14 product biosynthetic pathway constituents of a selected host microbial organism, the non-naturally occurring microbial organisms will include at least one exogenously expressed 6-aminocaproic acid, caprolactam, hexamethylenediamine or other C5-C14 product pathway-encoding nucleic acid and up to all encoding nucleic acids for one or more adipate, 6-aminocaproic acid, caprolactam, or other C5-C14 product biosynthetic pathways. For example, 6-aminocaproic acid, caprolactam, hexamethylenediamine or other C5-C14 product biosynthesis can be established in a host deficient in a pathway enzyme through exogenous expression of the corresponding encoding nucleic acid. In a host deficient in all enzymes of a 6-aminocaproic acid, caprolactam, hexamethylenediamine or other C5-C14 product pathway, exogenous expression of all enzymes in the pathway can be included, although it is understood that all enzymes of a pathway can be expressed even if the host contains at least one of the pathway enzymes.

Given the teachings and guidance provided herein, those skilled in the art will understand that the number of encoding nucleic acids to introduce in an expressible form will, at least, parallel the adipate, 6-aminocaproic acid, caprolactam, hexamethylenediamine or other C5-C14 product pathway deficiencies of the selected host microbial organism. Therefore, a non-naturally occurring microbial organism can have at least one, two, three, four, five, six, seven, eight, nine, ten, eleven or twelve, up to all nucleic acids encoding the above enzymes constituting a 6-aminocaproic acid, caprolactam, hexamethylenediamine or other C5-C14 product biosynthetic pathway. In some embodiments, the non-naturally occurring microbial organisms also can include other genetic modifications that facilitate or optimize 6-aminocaproic acid, caprolactam, hexamethylenediamine or other C5-C14 product biosynthesis or that confer other useful functions onto the host microbial organism. One such other functionality can include, for example, augmentation of the synthesis of one or more of the 6-aminocaproic acid, caprolactam, hexamethylenediamine or other C5-C14 product pathway precursors such as succinyl-CoA and/or acetyl-CoA in the case of adipate synthesis, or adipyl-CoA or adipate in the case of 6-aminocaproic acid or caprolactam synthesis, including the adipate pathway enzymes disclosed herein, or pyruvate and succinic semialdehyde, glutamate, glutaryl-CoA, homolysine or 2-amino-7-oxosubarate in the case of 6-aminocaprioate synthesis, or 6-aminocaproate, glutamate, glutaryl-CoA, pyruvate and 4-aminobutanal, or 2-amino-7-oxosubarate in the case of hexamethylenediamine synthesis.

A non-naturally occurring microbial organism can be generated from a host that contains the enzymatic capability to synthesize 6-aminocaproic acid, caprolactam, hexamethylenediamine or levulinic acid. Itt can be useful to increase the synthesis or accumulation of a 6-aminocaproic acid, caprolactam, hexamethylenediamine or other C5-C14 product pathway product to, for example, drive 6-aminocaproic acid, caprolactam, hexamethylenediamine or other C5-C14 product pathway reactions toward 6-aminocaproic acid, caprolactam, hexamethylenediamine or other C5-C14 product production. Increased synthesis or accumulation can be accomplished by, for example, overexpression of nucleic acids encoding one or more of the above-described 6-aminocaproic acid, caprolactam, hexamethylenediamine or other C5-C14 product pathway enzymes. Over expression of the 6-aminocaproic acid, caprolactam, hexamethylenediamine or other C5-C14 product pathway enzyme or enzymes can occur, for example, through exogenous expression of the endogenous gene or genes, or through exogenous expression of the heterologous gene or genes. Therefore, naturally occurring organisms can be readily generated to be non-naturally occurring microbial organisms, for example, producing 6-aminocaproic acid, caprolactam, hexamethylenediamine or other C5-C14 product, through overexpression of at least one, two, three, four, five, six, seven, eight, nine, ten, eleven, twelve, thirteen, fourteen, that is, up to all nucleic acids encoding 6-aminocaproic acid, caprolactam, hexamethylenediamine or other C5-C14 product biosynthetic pathway enzymes. In addition, a non-naturally occurring organism can be generated by mutagenesis of an endogenous gene that results in an increase in activity of an enzyme in the 6-aminocaproic acid, caprolactam, hexamethylenediamine or other C5-C14 product biosynthetic pathway.

Exogenous expression can confer the ability to custom tailor the expression and/or regulatory elements to the host and application to achieve a desired expression level that is controlled by the user. However, endogenous expression also can be utilized by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

A non-naturally occurring microbial organism can include one or more gene disruptions, where the organism produces a 6-ACA, caprolactam, HMDA, and/or other C5-C14 product. The disruptions occur in genes described herein so that the gene disruption reduces the activity of the gene product, such that the gene disruptions confer increased production of 6-ACA, caprolactam, HMDA, and/or other C5-C14 product onto the non-naturally occurring organism. Thus, a non-naturally occurring microbial organism, comprising one or more gene disruptions, the one or more gene disruptions described herein conferring increased production of 6-ACA, caprolactam, HMDA, and/or other C5-C14 product in the organism. As disclosed herein, such an organism contains a pathway for production of 6-ACA, caprolactam, HMDA, and/or other C5-C14 product.

It is understood that, in methods, any of the one or more exogenous nucleic acids can be introduced into a microbial organism to produce a non-naturally occurring microbial organism. The nucleic acids can be introduced so as to confer, for example, a 6-aminocaproic acid, caprolactam, hexamethylenediamine or other C5-C14 product biosynthetic pathway onto the microbial organism. Alternatively, encoding nucleic acids can be introduced to produce an intermediate microbial organism having the biosynthetic capability to catalyze some of the required reactions to confer 6-aminocaproic acid, caprolactam, hexamethylenediamine or other C5-C14 product biosynthetic capability. For example, a non-naturally occurring microbial organism having a 6-aminocaproic acid, caprolactam, hexamethylenediamine or other C5-C14 product biosynthetic pathway can comprise at least two exogenous nucleic acids encoding desired enzymes. In the case of adipate production, at least two exogenous nucleic acids can encode the enzymes such as the combination of succinyl-CoA: acetyl-CoA acyl transferase and 3-hydroxyacyl-CoA dehydrogenase, or succinyl-CoA: acetyl-CoA acyl transferase and 3-hydroxyadipyl-CoA dehydratransaminasee, or 3-hydroxyadipyl-CoA and adipate semialdehyde transaminase, or 3-hydroxyacyl-CoA and adipyl-CoA synthetase, and the like. In the case of caprolactam production, at least two exogenous nucleic acids can encode the enzymes such as the combination of CoA-dependent trans-enoyl-CoA reductase and transaminase, or CoA-dependent trans-enoyl-CoA reductransaminasee and amidohydrolase, or transaminase and amidohydrolase. In the case of 6-aminocaproic acid production, at least two exogenous nucleic acids can encode the enzymes such as the combination of an 4-hydroxy-2-oxoheptane-1,7-dioate (HODH) TAolase and a 2-oxohept-4-ene-1,7-dioate (OHED) hydratransaminasee, or a 2-oxohept-4-ene-1,7-dioate (OHED) hydratransaminasee and a 2-aminoheptane-1,7-dioate (2-AHD) decarboxylase, a 3-hydroxyadipyl-CoA dehydratransaminasee and a adipyl-CoA dehydrogenase, a glutamyl-CoA transferase and a 6-aminopimeloyl-CoA hydrolase, or a glutaryl-CoA beta-ketothiolase and a 3-aminopimelate 2,3-aminomutransaminasee. In the case of hexamethylenediamine production, at least two exogenous nucleic acids can encode the enzymes such as the combination of 6-aminocaproate kinase and [(6-aminohexanoyl)oxy]phosphonate (6-AHOP) oxidoreductransaminasee, or a 6-acetamidohexanoate kinase and an [(6-acetamidohexanoyl)oxy]phosphonate (6-AAHOP) oxidoreductransaminasee, 6-aminocaproate N-acetyltransferase and 6-acetamidohexanoyl-CoA oxidoreductransaminasee, a 3-hydroxy-6-aminopimeloyl-CoA dehydratransaminasee and a 2-amino-7-oxoheptanoate aminotransferase, or a 3-oxopimeloyl-CoA ligase and a homolysine decarboxylase. Thus, it is understood that any combination of two or more enzymes of a biosynthetic pathway can be included in a non-naturally occurring microbial organism.

Similarly, it is understood that any combination of three or more enzymes of a biosynthetic pathway can be included in a non-naturally occurring microbial organism, for example, in the case of adipate production, the combination of enzymes succinyl-CoA: acetyl-CoA acyl transferase, 3-hydroxyacyl-CoA dehydrogenase, and 3-hydroxyadipyl-CoA dehydratransaminasee; or succinyl-CoA: acetyl-CoA acyl transferase, 3-hydroxyacyl-CoA dehydrogenase andadipate semialdehydereductransaminasee; or succinyl-CoA: acetyl-CoA acyl transferase, 3-hydroxyacyl-CoA dehydrogenase and adipyl-CoA synthetransaminasee; or 3-hydroxyacyl-CoA dehydrogenase, 3-hydroxyadipyl-CoA dehydratransaminasee and adipyl-CoA: acetyl-CoA transferase, and so forth, as desired, so long as the combination of enzymes of the desired biosynthetic pathway results in production of the corresponding desired product. In the case of 6-aminocaproic acid production, the at least three exogenous nucleic acids can encode the enzymes such as the combination of an 4-hydroxy-2-oxoheptane-1,7-dioate (HODH) TAolase, a 2-oxohept-4-ene-1,7-dioate (OHED) hydratransaminasee and a 2-oxoheptane-1,7-dioate (2-OHD) decarboxylase, or a 2-oxohept-4-ene-1,7-dioate (OHED) hydratransaminasee, a 2-aminohept-4-ene-1,7-dioate (2-AHE) reductransaminasee and a 2-aminoheptane-1,7-dioate (2-AHD) decarboxylase, or a 3-hydroxyadipyl-CoA dehydratransaminasee, 2,3-dehydroadipyl-CoA reductransaminasee and a adipyl-CoA dehydrogenase, or a 6-amino-7-carboxyhept-2-enoyl-CoA reductransaminasee, a 6-aminopimeloyl-CoA hydrolase and a 2-aminopimelate decarboxylase, or a glutaryl-CoA beta-ketothiolase, a 3-aminating oxidoreductransaminasee and a 2-aminopimelate decarboxylase, or a 3-oxoadipyl-CoA thiolase, a 5-carboxy-2-pentenoate reductransaminasee and a adipate reductransaminasee. In the case of hexamethylenediamine production, at least three exogenous nucleic acids can encode the enzymes such as the combination of 6-aminocaproate kinase, [(6-aminohexanoyl)oxy]phosphonate (6-AHOP) oxidoreductransaminasee and 6-aminocaproic semialdehyde aminotransferase, or a 6-aminocaproate N-acetyltransferase, a 6-acetamidohexanoate kinase and an [(6-acetamidohexanoyl)oxy]phosphonate (6-AAHOP) oxidoreductransaminasee, or 6-aminocaproate N-acetyltransferase, a [(6-acetamidohexanoyl)oxy]phosphonate (6-AAHOP) acyltransferase and 6-acetamidohexanoyl-CoA oxidoreductransaminasee, or a 3-oxo-6-aminopimeloyl-CoA oxidoreductransaminasee, a 3-hydroxy-6-aminopimeloyl-CoA dehydratransaminasee and a homolysine decarboxylase, or a 2-oxo-4-hydroxy-7-aminoheptanoate TAolase, a 2-oxo-7-aminohept-3-enoate reductransaminasee and a homolysine decarboxylase, or a 6-acetamidohexanoate reductransaminasee, a 6-acetamidohexanal aminotransferase and a 6-acetamidohexanamine N-acetyltransferase. Similarly, any combination of four or more enzymes of a biosynthetic pathway as disclosed herein can be included in a non-naturally occurring microbial organism, as desired, so long as the combination of enzymes of the desired biosynthetic pathway results in production of the corresponding desired product.

In addition to the biosynthesis of 6-aminocaproic acid, caprolactam, hexamethylenediamine or other C5-C14 product as described herein, the non-naturally occurring microbial organisms and methods also can be utilized in various combinations with each other and with other microbial organisms and methods well known in the art to achieve product biosynthesis by other routes. For example, one alternative to produce 6-aminocaproic acid, caprolactam, hexamethylenediamine or other C5-C14 product other than use of the 6-aminocaproic acid, caprolactam, hexamethylenediamine or other C5-C14 product producers is through addition of another microbial organism capable of converting an adipate, 6-aminocaproic acid or caprolactam pathway intermediate to 6-aminocaproic acid, caprolactam, hexamethylenediamine or other C5-C14 product. One such procedure includes, for example, the fermentation of a microbial organism that produces a 6-aminocaproic acid, caprolactam, hexamethylenediamine or other C5-C14 product pathway intermediate. The 6-aminocaproic acid, caprolactam, hexamethylenediamine or other C5-C14 product pathway intermediate can then be used as a substrate for a second microbial organism that converts the 6-aminocaproic acid, caprolactam, hexamethylenediamine or other C5-C14 product pathway intermediate to 6-aminocaproic acid, caprolactam, hexamethylenediamine or other C5-C14 product. The 6-aminocaproic acid, caprolactam, hexamethylenediamine or other C5-C14 product pathway intermediate can be added directly to another culture of the second organism or the original culture of 6-aminocaproic acid, caprolactam, hexamethylenediamine or other C5-C14 product pathway intermediate producers can be depleted of these microbial organisms by, for example, cell separation, and then subsequent addition of the second organism to the fermentation broth can be utilized to produce the final product without intermediate purification steps.

The non-naturally occurring microbial organisms and methods can be assembled in a wide variety of sub pathways to achieve biosynthesis of, for example, 6-aminocaproic acid, caprolactam, hexamethylenediamine or other C5-C14 product. The biosynthetic pathways for a desired product can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of 6-aminocaproic acid, caprolactam, hexamethylenediamine or other C5-C14 product can be accomplished by constructing a microbial organism that contains biosynthetic pathways for conversion of one pathway intermediate to another pathway intermediate or the product. Alternatively, 6-aminocaproic acid, caprolactam, hexamethylenediamine or other C5-C14 product also can be biosynthetically produced from microbial organisms through co-culture or co-fermentation using two organisms in the same vessel, where the first microbial organism produces a 6-aminocaproic acid, caprolactam, hexamethylenediamine or other C5-C14 product intermediate and the second microbial organism converts the intermediate to 6-aminocaproic acid, caprolactam, hexamethylenediamine or other C5-C14 product.

Given the teachings and guidance provided herein, those skilled in the art will understand that a wide variety of combinations and permutations exist for the non-naturally occurring microbial organisms and methods together with other microbial organisms, with the co-culture of other non-naturally occurring microbial organisms having sub pathways and with combinations of other chemical and/or biochemical procedures well known in the art to produce 6-aminocaproic acid, caprolactam, hexamethylenediamine or other C5-C14 product.

Similarly, it is understood by those skilled in the art that a host organism can be selected based on desired characteristics for introduction of one or more gene disruptions to increase production of 6-aminocaproic acid, caprolactam, hexamethylenediamine or other C5-C14 product. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods can be applied to any suitable host microorganism to identify the cognate metabolic alterations needed to construct an organism in a species of interest that will increase 6-aminocaproic acid, caprolactam, hexamethylenediamine or other C5-C14 product biosynthesis. The increased production can couple biosynthesis of 6-aminocaproic acid, caprolactam, hexamethylenediamine or other C5-C14 product to growth of the organism, and can obligatorily couple production of 6-aminocaproic acid, caprolactam, hexamethylenediamine or other C5-C14 product to growth of the organism if desired.

Sources of encoding nucleic acids for a 6-aminocaproic acid, caprolactam, hexamethylenediamine or other C5-C14 product pathway enzyme can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. The source of the encoding nucleic acids for a 6-aminocaproic acid, caprolactam, hexamethylenediamine or other C5-C14 product pathway enzyme can be shown in Table 4. The source of the encoding nucleic acids for a 6-aminocaproic acid, caprolactam, hexamethylenediamine or other C5-C14 product pathway enzyme are species such as, Escherichia coli, Escherichia coli str. K12, Escherichia coli C, Escherichia coli W, Pseudomonas sp, Pseudomonas knackmussii, Pseudomonas sp. Strain B13, Pseudomonas putida, Pseudomonas fluorescens, Pseudomonas stutzeri, Pseudomonas mendocina, Rhodopseudomonas palustris, Mycobacterium tuberculosis, Vibrio cholera, Heliobacter pylori, Klebsiella pneumoniae, Serratia proteamaculans, Streptomyces sp. 2065, Pseudomonas aeruginosa, Pseudomonas aeruginosa PAO1, Ralstonia eutropha, Ralstonia eutropha H16, Clostridium acetobutylicum, Euglena gracilis, Treponema denticola, Clostridium kluyveri, Homo sapiens, Rattus norvegicus, Acinetobacter sp. ADP1, Acinetobacter sp. Strain M-1, Streptomyces coelicolor, Eubacterium barkeri, Peptostreptococcus asaccharolyticus, Clostridium botulinum, Clostridium botulinum A3 str, Clostridium tyrobutyricum, Clostridium pasteurianum, Clostridium thermoaceticum (Moorella thermoaceticum), Moorella thermoacetica Acinetobacter calcoaceticus, Mus musculus, Sus scrofa, Flavobacterium sp, Arthrobacter aurescens, Penicillium chrysogenum, Aspergillus niger, Aspergillus nidulans, Bacillus subtilis, Saccharomyces cerevisiae, Zymomonas mobilis, Mannheimia succiniciproducens, Clostridium ljungdahlii, Clostridium carboxydivorans, Geobacillus stearothermophilus, Agrobacterium tumefaciens, Achromobacter xylosoxidans, Achromobacter denitrificans, Arabidopsis thaliana, Haemophilus influenzae, Acidaminococcus fermentans, Clostridium sp. M62/1, Fusobacterium nucleatum, Bos taurus, Zoogloea ramigera, Rhodobacter sphaeroides, Clostridium beijerinckii, Metallosphaera sedula, Thermoanaerobacter species, Thermoanaerobacter brockii, Acinetobacter baylyi, Porphyromonas gingivalis, Leuconostoc mesenteroides, Sulfolobus tokodaii, Sulfolobus tokodaii 7, Sulfolobus solfataricus, Sulfolobus solfataricus, Sulfolobus acidocaldarius, Salmonella typhimurium, Salmonella enterica, Thermotoga maritima, Halobacterium salinarum, Bacillus cereus, Clostridium difficile, Alkaliphilus metalliredigenes, Thermoanaerobacter tengcongensis, Saccharomyces kluyveri, Helicobacter pylori, Corynebacterium glutamicum, Clostridium saccharoperbutylacetonicum, Pseudomonas chlororaphis, Streptomyces clavuligerus, Campylobacter jejuni, Thermus thermophilus, Pelotomaculum thermopropionicum, Bacteroides capillosus, Anaerotruncus colihominis, Natranaerobius thermophilius, Archaeoglobus fulgidus, Archaeoglobus fulgidus DSM 4304, Haloarcula marismortui, Pyrobaculum aerophilum, Pyrobaculum aerophilum str. IM2, Nicotiana tabacum, Menthe piperita, Pinus taeda, Hordeum vulgare, Zea mays, Rhodococcus opacus, Cupriavidus necator, Bradyrhizobium japonicum, Bradyrhizobium japonicum USDA110, Ascarius suum, butyrate-producing bacterium L2-50, Bacillus megaterium, Methanococcus maripaludis, Methanosarcina mazei, Methanosarcina mazei, Methanocarcina barkeri, Methanocaldococcus jannaschii, Caenorhabditis elegans, Leishmania major, Methylomicrobium alcaliphilum 20Z, Chromohalobacter salexigens, Archaeglubus fulgidus, Chlamydomonas reinhardtii, Trichomonas vaginalis G3, Trypanosoma brucei, Mycoplana ramose, Micrococcus luteas, Acetobacter pasteurians, Kluyveromyces lactis, Mesorhizobium loti, Lactococcus lactis, Lysinibacillus sphaericus, Candida boidinii, Candida albicans SC5314, Burkholderia ambifaria AMMD, Ascaris suun, Acinetobacter baumanii, Acinetobacter calcoaceticus, Burkholderia phymatum, Candida albicans, Clostridium subterminale, Cupriavidus taiwanensis, Flavobacterium lutescens, Lachancea kluyveri, Lactobacillus sp. 30a, Leptospira interrogans, Moorella thermoacetica, Myxococcus xanthus, Nicotiana glutinosa, Nocardia iowensis (sp. NRRL 5646), Pseudomonas reinekei MT1, Ralstonia eutropha JMP134, Ralstonia metallidurans, Rhodococcus jostii, Schizosaccharomyces pombe, Selenomonas ruminantium, Streptomyces clavuligenus, Syntrophus aciditrophicus, Vibrio parahaemolyticus, Vibrio vulnificus, as well as other exemplary species disclosed herein or available as source organisms for corresponding genes (see Examples). However, with the complete genome sequence available for now more than 550 species (with more than half of these available on public databases such as the NCBI), including 395 microorganism genomes and a variety of yeast, fungi, plant, and mammalian genomes, the identification of genes encoding the requisite 6-aminocaproic acid, caprolactam, hexamethylenediamine or other C5-C14 product biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations enabling biosynthesis of 6-aminocaproic acid, caprolactam, hexamethylenediamine or other C5-C14 product described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when a 6-aminocaproic acid, caprolactam, hexamethylenediamine or other C5-C14 product biosynthetic pathway exists in an unrelated species, 6-aminocaproic acid, caprolactam, hexamethylenediamine or other C5-C14 product biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods can be applied to all microbial organisms using the cognate metabolic alterations to those exemplified herein to construct a microbial organism in a species of interest that will synthesize 6-aminocaproic acid, caprolactam, hexamethylenediamine or other C5-C14 product.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and the like. For example, E. coli is a particularly useful host organism since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Methods for constructing and testing the expression levels of a non-naturally occurring 6-aminocaproic acid, caprolactam, hexamethylenediamine or levulinic acid-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

Exogenous nucleic acid sequences involved in a pathway for production of 6-aminocaproic acid, caprolactam, hexamethylenediamine or other C5-C14 product can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more 6-aminocaproic acid, caprolactam, hexamethylenediamine or levulinic acid biosynthetic pathway encoding nucleic acids as exemplified herein operably linked to expression control sequences functional in the host organism. Expression vectors applicable for use in the microbial host organisms include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoTransaminase, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

In some embodiments are methods for producing a desired intermediate or product such as adipate, 6-aminocaproic acid, caprolactam, hexamethylenediamine or other C5-C14 product. For example, a method for producing adipate can involve culturing a non-naturally occurring microbial organism having an adipate pathway, the pathway including at least one exogenous nucleic acid encoding an adipate pathway enzyme expressed in a sufficient amount to produce adipate, under conditions and for a sufficient period of time to produce adipate, the adipate pathway including succinyl-CoA: acetyl-CoA acyl transferase, 3-hydroxyacyl-CoA dehydrogenase, 3-hydroxyadipyl-CoA dehydratransaminasee, adipate semialdehydereductransaminasee, and adipyl-CoA synthetransaminasee or phosphotransadipylase/adipate kinase or adipyl-CoA: acetyl-CoA transferase or adipyl-CoA hydrolase. Additionally, a method for producing adipate can involve culturing a non-naturally occurring microbial organism having an adipate pathway, the pathway including at least one exogenous nucleic acid encoding an adipate pathway enzyme expressed in a sufficient amount to produce adipate, under conditions and for a sufficient period of time to produce adipate, the adipate pathway including succinyl-CoA: acetyl-CoA acyl transferase, 3-oxoadipyl-CoA transferase, 3-oxoadipate reductransaminasee, 3-hydroxyadipate dehydratransaminasee, and 2-enoate reductransaminasee.

Further, a method for producing 6-aminocaproic acid can involve culturing a non-naturally occurring microbial organism having a 6-aminocaproic acid pathway, the pathway including at least one exogenous nucleic acid encoding a 6-aminocaproic acid pathway enzyme expressed in a sufficient amount to produce 6-aminocaproic acid, under conditions and for a sufficient period of time to produce 6-aminocaproic acid, the 6-aminocaproic acid pathway including CoA-dependent trans-enoyl-CoA reductransaminasee and transaminase or 6-aminocaproate dehydrogenase. Additionally, a method for producing caprolactam can involve culturing a non-naturally occurring microbial organism having a caprolactam pathway, the pathway including at least one exogenous nucleic acid encoding a caprolactam pathway enzyme expressed in a sufficient amount to produce caprolactam, under conditions and for a sufficient period of time to produce caprolactam, the caprolactam pathway including CoA-dependent aldehyde dehydrogenase, transaminase or 6-aminocaproate dehydrogenase, and amidohydrolase.

Suitable purification and/or assays to test for the production of 6-aminocaproic acid, caprolactam, hexamethylenediamine or levulinic acid can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme activities from the exogenous DNA sequences can also be assayed using methods well known in the art.

The 6-aminocaproic acid, caprolactam, hexamethylenediamine or levulinic acid can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration.

Any of the non-naturally occurring microbial organisms described herein can be cultured to produce and/or secrete the biosynthetic products. For example, the 6-aminocaproic acid, caprolactam, hexamethylenediamine or other C5-C14 product producers can be cultured for the biosynthetic production of 6-aminocaproic acid, caprolactam, hexamethylenediamine or other C5-C14 product.

For the production of 6-aminocaproic acid, caprolactam, hexamethylenediamine or other C5-C14 product, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in U.S. Pat. No. 7,947,483 issued May 24, 2011. Fermentations can be performed in a batch, fed-batch or continuous manner, as disclosed herein.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring microorganism. Such sources include, for example, sugars such as glucose, xylose, arabinose, galactose, mannose, fructose, sucrose and starch. Other sources of carbohydrate include, for example, renewable feedstocks and biomass. Exemplary types of biomasses that can be used as feedstocks in the methods include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms for the production of 6-aminocaproic acid, caprolactam, hexamethylenediamine or other C5-C14 product.

In addition to renewable feedstocks such as those exemplified above, the 6-aminocaproic acid, caprolactam, hexamethylenediamine, other C5-C14 product microbial organisms also can be modified for growth on syngas as its source of carbon. One or more proteins or enzymes can be expressed in the 6-aminocaproic acid, caprolactam, hexamethylenediamine or other C5-C14 product producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H2 and CO and can be obtained from the gasification of any organic feedstock, including but not limited to coal, coal oil, natural gas, biomass, and waste organic matter. Gasification is generally carried out under a high fuel to oxygen ratio. Although largely H2 and CO, syngas can also include CO2 and other gases in smaller quantities. Thus, synthesis gas provides a cost effective source of gaseous carbon such as CO and additionally, CO2.

The Wood-Ljungdahl pathway catalyzes the conversion of CO and H2 to acetyl-CoA and other products such as acetate. Organisms capable of utilizing CO and syngas also generally have the capability of utilizing C02 and C02/H2 mixtures through the same basic set of enzymes and transformations encompassed by the Wood-Ljungdahl pathway. H2-dependent conversion of C02 to acetate by microorganisms was recognized long before it was revealed that CO also could be used by the same organisms and that the same pathways were involved. Many acetogens have been shown to grow in the presence of C02 and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2 CO2+4 H2+n ADP+n Pi→CH3COOH+2 H2O+n ATP

Hence, non-naturally occurring microorganisms possessing the Wood-Ljungdahl pathway can utilize CO2 and H2 mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes: ferredoxin oxidoreductransaminasee, formate dehydrogenase, formyltetrahydrofolate synthetransaminasee, methenyltetrahydrofolate cyclodehydratransaminasee, methylenetetrahydrofolate dehydrogenase and methylenetetrahydrofolate reductransaminasee. The reactions in the carbonyl branch are catalyzed in order by the following enzymes or proteins: cobalamide corrinoid/iron-sulfur protein, methyltransferase, carbon monoxide dehydrogenase, acetyl-CoA synthase, acetyl-CoA synthase disulfide reductransaminasee and hydrogenase, and these enzymes can also be referred to as methyltetrahydrofolate: corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate a 6-aminocaproic acid, caprolactam, hexamethylenediamine or levulinic acid pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms such that the modified organism contains the complete Wood-Ljungdahl pathway will confer syngas utilization ability.

Additionally, the reductive (reverse) tricarboxylic acid cycle coupled with carbon monoxide dehydrogenase and/or hydrogenase activities can also be used for the conversion of CO, CO2 and/or H2 to acetyl-CoA and other products such as acetate. Organisms capable of fixing carbon via the reductive TCA pathway can utilize one or more of the following enzymes: ATP citrate-lyase, citrate lyase, aconitransaminasee, isocitrate dehydrogenase, alpha-ketoglutarate: ferredoxin oxidoreductransaminasee, succinyl-CoA synthetransaminasee, succinyl-CoA transferase, fumarate reductransaminasee, fumarase, malate dehydrogenase, NAD(P)Ferredoxin oxidoreductransaminasee, carbon monoxide dehydrogenase, and hydrogenase. Specifically, the reducing equivalents extracted from CO and/or H2 by carbon monoxide dehydrogenase and hydrogenase are utilized to fix CO2 via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetransaminasee. Acetyl-CoA can be converted to the p-toluate, terepathalate, or (2-hydroxy-3-methyl-4-oxobutoxy) phosphonate precursors, glyceraldehyde-3-phosphate, phosphoenolpyruvate, and pyruvate, by pyruvate: ferredoxin oxidoreductransaminasee and the enzymes of gluconeogenesis. Following the teachings and guidance provided herein for introducing a sufficient number of encoding nucleic acids to generate a p-toluate, terephthalate or (2-hydroxy-3-methyl-4-oxobutoxy) phosphonate pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms such that the modified organism contains the complete reductive TCA pathway will confer syngas utilization ability.

Given the teachings and guidance provided herein, those skilled in the art will understand that a non-naturally occurring microbial organism can be produced that secretes the biosynthesized compounds when grown on a carbon source such as a carbohydrate. Such compounds include, for example, 6-aminocaproic acid, caprolactam, hexamethylenediamine or other C5-C14 product and any of the intermediate metabolites in the 6-aminocaproic acid, caprolactam, hexamethylenediamine or other C5-C14 product pathway. All that is required is to engineer in one or more of the required enzyme activities to achieve biosynthesis of the desired compound or intermediate including, for example, inclusion of some or all of the 6-aminocaproic acid, caprolactam, hexamethylenediamine or other C5-C14 product biosynthetic pathways. Accordingly, some embodiments provide a non-naturally occurring microbial organism that produces and/or secretes 6-aminocaproic acid, caprolactam, hexamethylenediamine or other C5-C14 product when grown on a carbohydrate and produces and/or secretes any of the intermediate metabolites shown in the 6-aminocaproic acid, caprolactam, hexamethylenediamine or other C5-C14 product pathway when grown on a carbohydrate. For example, an adipate producing microbial organisms can initiate synthesis from an intermediate, for example, 3-oxoadipyl-CoA, 3-hydroxyadipyl-CoA, 5-carboxy-2-pentenoyl-CoA, or adipyl-CoA (see FIG. 1), as desired. In addition, an adipate producing microbial organism can initiate synthesis from an intermediate, for example, 3-oxoadipyl-CoA, 3-oxoadipate, 3-hydroxyadipate, or hexa-2-enedioate. The 6-aminocaproic acid producing microbial organism can initiate synthesis from an intermediate, for example, adipate semialdehyde. The caprolactam producing microbial organism can initiate synthesis from an intermediate, for example, adipate semialdehyde or 6-aminocaproic acid (see FIG. 1), as desired.

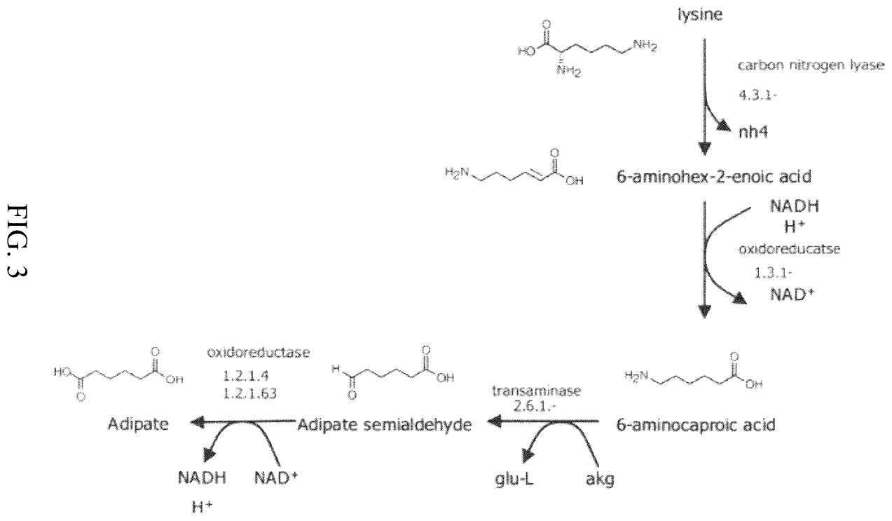

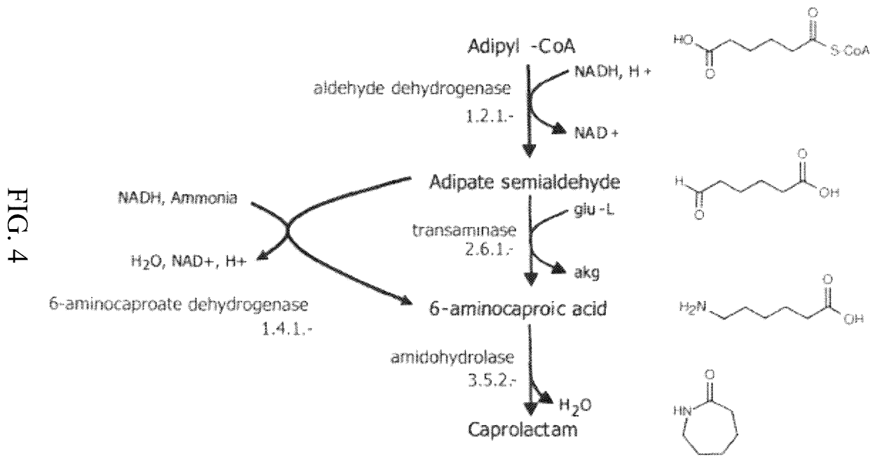

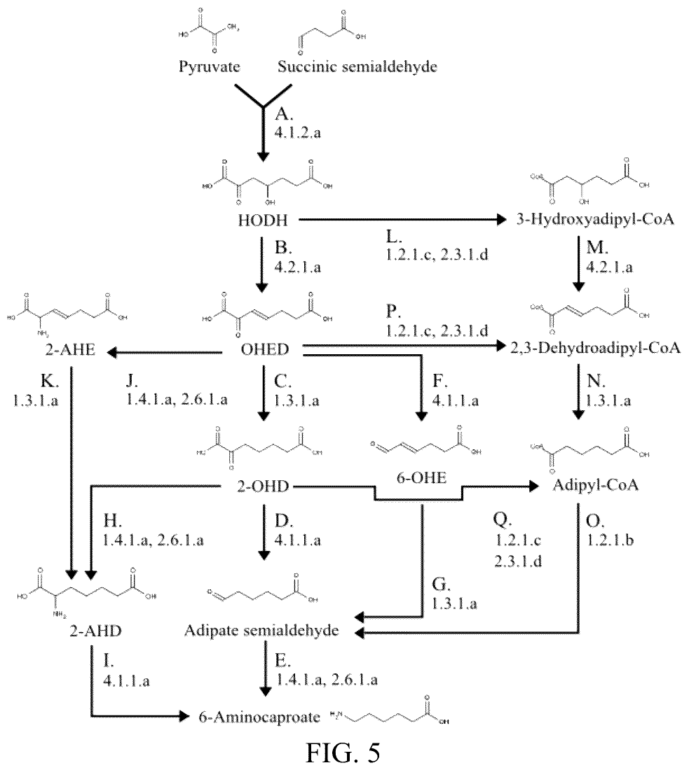

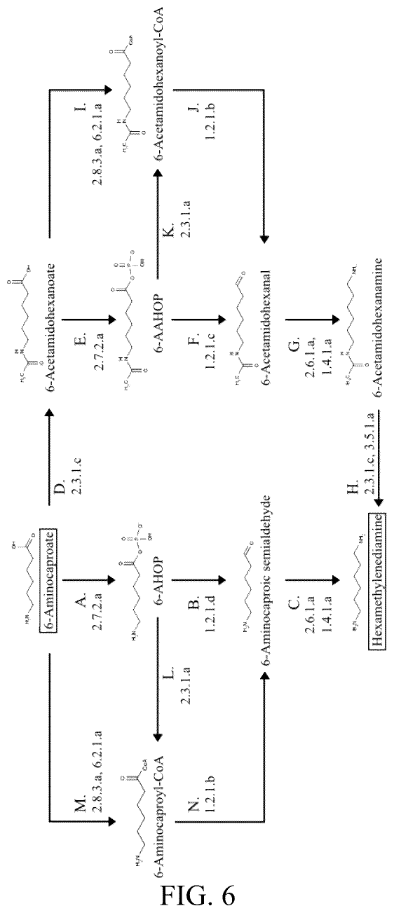

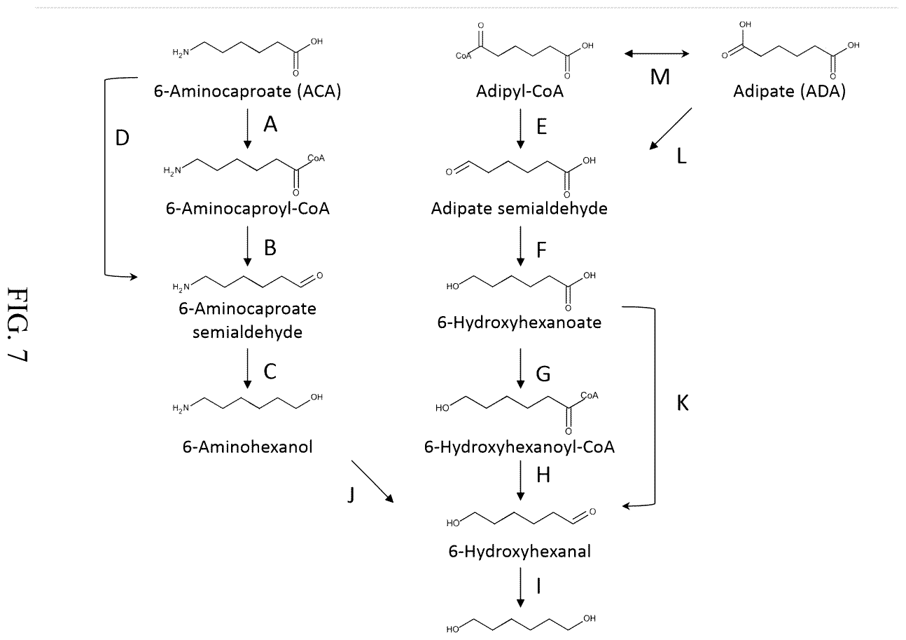

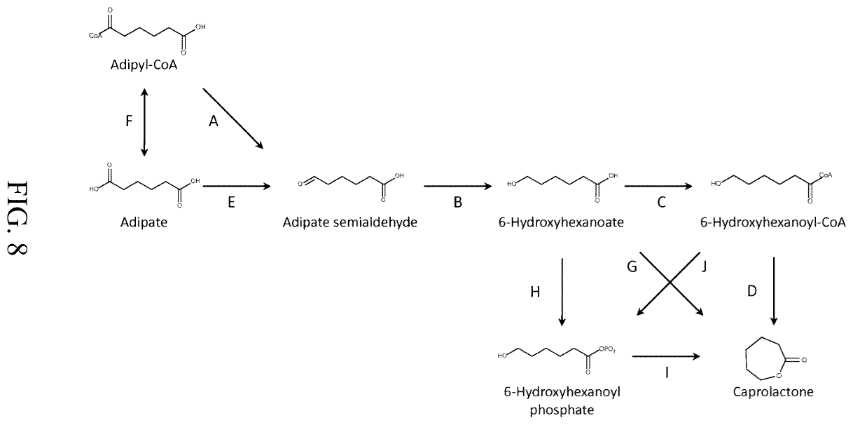

In some embodiments, the non-naturally occurring microorganisms can generate adipate, 6ACA, caprolactone, hexamethyelenediamine or caproclactam as shown in FIG. 3-8.

The non-naturally occurring microbial organisms can further include an exogenously expressed nucleic acid encoding an aldehyde dehydrognease (ALD) or a transenoyl reductase (TER) or both. The ALD reacts with adipyl-CoA to produce adipate semialdehyde, whereas the TER reacts with 5-carboxy-2-pentenoyl-CoA (CPCoA) to form adipyl CoA.

The ALD enzymes have greater catalytic efficiency and activity for the adipyl CoA substrate as compared to succinyl-CoA, or acetyl-CoA, or both substrates. Exemplary ALD enzymes are as shown in Table 10.

TABLE 10
Activity of Aldehyde Dehydrogenases on Adipyl-CoA

| | | Accession | Activity - | Activity - |
|---|---|---|---|---|
| NO. | Organism | No/SEQ ID NO. | NADH | NADPH |
| 1 | Clostridium kluyveri DSM555 | SEQ ID NO: 141 | − | + |
| 2 | Porphyromonas gingivalis W83 | SEQ ID NO: 142 | + | − |
| 3 | Clostridium difficile 630 | SEQ ID NO: 143 | − | + |
| 4 | Kluyvera intestini | WP_071196317.1 | + | − |
| 5 | Clostridium neonatale | WP_058295546.1 | − | − |
| 6 | Aerococcus sp. HMSC062B07 | WP_070558456.1 | − | − |
| 7 | Peptostreptococcaceae bacterium | WP_021676458.1 | + | − |
| | oral | | | |
| 8 | Dasania marina | WP_026244399.1 | − | − |
| 9 | Porphyromonadaceae bacterium | WP_036830068.1 | − | − |
| | COT-184 | | | |
| 10 | Clostridium lundense | WP_027623222.1 | − | − |
| 11 | Anaerocolumna jejuensis | WP_073279774.1 | + | − |
| 12 | Clostridium homopropionicum | WP_052222510.1 | − | − |
| 13 | Geosporobacter ferrireducens | WP_069981616.1 | − | − |
| 14 | Listeria ivanovii | WP_038407128.1 | − | − |
| 15 | Bacillus soli | WP_066062455.1 | + | − |
| 16 | Enterococcus rivorum | WP_069697141.1 | − | − |
| 17 | Desnuesiella massiliensis | WP_055665162.1 | + | − |
| 18 | Bacteroidales bacterium KA00251 | WP_066041885.1 | − | − |
| 19 | Caldanaerobius | WP_026487268.1 | + | − |
| | polysaccharolyticus | | | |
| 20 | Clostridium sp. ASF356 | WP_004036483.1 | − | − |
| 21 | Clostridiales bacterium DRI-13 | WP_034420506.1 | − | − |
| 22 | Fusobacterium ulcerans ATCC | WP_005981617.1 | − | − |
| | 49185 | | | |
| 23 | Anaerocolumna jejuensis | WP_073279351.1 | − | − |
| 24 | Cellulosilyticum sp. I15G10I2 | WP_070001026.1 | + | − |
| 25 | Geosporobacter ferrireducens | WP_083273866.1 | + | − |
| 26 | Pelosinus sp. UFO1 | WP_038668911.1 | − | − |
| 27 | Bacillus korlensis | WP_084362095.1 | + | − |
| 28 | Acidaminococcus massiliensis | WP_075579339.1 | + | − |
| 29 | Eubacterium sp. SB2 | WP_050640767.1 | − | − |
| 30 | Erwinia teleogrylli | WP_058911295.1 | − | − |
| 31 | Lachnospiraceae bacterium 32 | WP_016223553.1 | + | − |
| 32 | Eubacterium plexicaudatum | WP_004061597.1 | + | − |
| 33 | Clostridium sp. KNHs205 | WP_033166114.1 | + | − |
| 34 | Butyricimonas virosa | WP_027200274.1 | − | − |
| 35 | Malonomonas rubra | WP_072908980.1 | − | − |
| 36 | Robinsoniella peoriensis | WP_044292972.1 | + | − |
| 37 | Clostridium taeniosporum | WP_069679818.1 | − | − |
| 38 | Caldithrix abyssi | WP_006928331.1 | + | − |
| 39 | Piscicoccus intestinalis | WP_084343789.1 | − | − |
| 40 | Sporomusa sphaeroides | WP_075753933.1 | + | − |
| 41 | Bacillus sp. FJAT-25547 | WP_057762439.1 | + | − |
| 42 | Dorea sp. D27 | WP_049729435.1 | + | − |
| 43 | Oscillibacter sp. 13 | WP_081646270.1 | − | − |
| 44 | Enterococcus phoeniculicola | WP_010767571.1 | + | − |
| 45 | Blautia schinkii | WP_044941637.1 | + | − |
| 46 | Shuttleworthia satelles DSM 14600 | WP_006905683.1 | − | − |
| 47 | Clostridium intestinale | WP_073018444.1 | + | − |
| 48 | Massilioclostridium coli | WP_069989048.1 | − | − |
| 49 | Cloacibacillus porcorum | WP_066745012.1 | − | − |
| 50 | Clostridium sp. CL-2 | WP_032120205.1 | − | − |
| 51 | Clostridia bacterium UC5.1-1D10 | WP_054330586.1 | − | − |
| 52 | Methylobacterium sp. CCH5-D2 | WP_082772960.1 | − | − |
| 53 | Sporosarcina globispora | WP_053435653.1 | + | + |
| 54 | Lachnospiraceae bacterium | WP_031546337.1 | − | − |
| | AC3007 | | | |
| 55 | Lachnospiraceae bacterium 28-4 | WP_016290199.1 | − | − |
| 56 | Enterococcus avium | WP_034875865.1 | − | − |
| 57 | Desulfotomaculum | WP_027356260.1 | − | − |
| | thermocisternum | | | |
| 58 | Rhodobacter aestuarii | WP_076486054.1 | + | − |
| 59 | Clostridium grantii | WP_073337420.1 | + | − |
| 60 | Collinsella sp. GD7 | WP_066830323.1 | + | − |
| 61 | Clostridium estertheticum | WP_071611886.1 | − | − |
| 62 | bacterium MS4 | WP_038325413.1 | − | − |
| 63 | Clostridium glycyrrhizinilyticum | WP_009268007.1 | + | − |
| 64 | Bacillus horikoshii | WP_082892049.1 | − | − |
| 65 | Thermincola ferriacetica | WP_052218568.1 | + | − |
| 66 | Lachnospiraceae bacterium | WP_035653923.1 | + | − |
| | AC3007 | | | |
| 67 | Eubacterium sp. 14-2 | WP_016216571.1 | + | − |
| 68 | Candidatus Marispirochaeta | WP_069895590.1 | − | − |
| | associata | | | |
| 69 | Clostridium drakei | WP_032078293.1 | − | − |
| 70 | Halanaerobium kushneri | WP_076543773.1 | − | − |
| 71 | Clostridium fallax | WP_072896506.1 | − | − |
| 72 | Flavonifractor plautii | WP_009261118.1 | − | − |
| 73 | Clostridium propionicum | WP_066049640.1 | − | − |
| 74 | Anaerosalibacter massiliensis | WP_042682918.1 | + | − |
| 75 | Clostridium indolis DSM 755 | WP_024295710.1 | + | − |
| 76 | Gabonibacter massiliensis | WP_059027034.1 | − | − |
| 77 | Catabacter hongkongensis | WP_046444791.1 | + | + |
| 78 | Desulfitibacter alkalitolerans | WP_028307735.1 | − | − |
| 79 | Porphyromonas levii | WP_018357742.1 | − | − |
| 80 | Bacillus thermotolerans | WP_039235348.1 | + | − |
| 81 | Desulfitibacter alkalitolerans | WP_028307055.1 | − | − |
| 82 | Gracilibacillus kekensis | WP_073203236.1 | + | + |
| 83 | Lactonifactor longoviformis | WP_072848455.1 | − | − |
| 84 | Propionispora sp. 2/2-37 | WP_054258533.1 | + | − |
| 85 | Erysipelothrix larvae | WP_067632640.1 | − | − |
| 86 | Clostridium chauvoei | WP_021875658.1 | + | − |
| 87 | Thermoanaerobacterium | WP_014757178.1 | + | − |
| | aotearoense | | | |
| 88 | Ruminococcus sp. AT10 | WP_059066688.1 | + | − |
| 89 | Porphyromonas sp. HMSC077F02 | WP_070707924.1 | − | − |
| 90 | Acetobacterium dehalogenans | WP_026396046.1 | + | − |
| 91 | Spirochaeta alkalica | WP_018526526.1 | + | − |
| 92 | Alistipes sp. ZOR0009 | WP_047449305.1 | − | − |
| 93 | Clostridiisalibacter paucivorans | WP_026895448.1 | − | − |
| 94 | Clostridium caminithermale DSM | WP_073149471.1 | + | + |
| | 15212 | | | |
| 95 | Caldanaerobius fijiensis | WP_073341480.1 | + | − |
| 96 | Clostridium kluyveri | WP_073539833.1 | − | − |
| 97 | Pelosinus fermentans | WP_007958399.1 | + | − |
| 98 | Halanaerobium saccharolyticum | WP_005487288.1 | − | − |
| | subsp. saccharolyticum DSM 6643 | | | |
| 99 | Anaeroarcus burkinensis DSM | WP_018702299.1 | − | − |
| | 6283 | | | |
| 100 | Blautia wexlerae | WP_026648408.1 | + | − |
| 101 | Paenibacillus sp. OSY-SE | WP_019424162.1 | + | − |
| 102 | Brachyspira intermedia PWSA | WP_014488056.1 | − | − |
| 103 | Spirochaetes bacterium | OHD32879.1 | + | − |
| | GWC2_52_13 | | | |
| 104 | Thermoanaerobacterales bacterium | KUK31085.1 | − | − |
| | 50_218 | | | |
| 105 | Cohaesibacter marisflavi | WP_090072157.1 | − | − |
| 106 | Gracilibacillus ureilyticus | WP_089739945.1 | − | − |
| 107 | Romboutsia lituseburensis DSM | WP_092724914.1 | + | − |
| 108 | uncultured Clostridium sp. | SCJ29526.1 | − | − |
| 109 | Clostridium sp. CAG: 448 | CDC62685.1 | + | − |
| 110 | Clostridium ultunense Esp | CCQ95129.1 | − | − |
| 111 | Yersinia bercovieri ATCC 43970 | WP_005274635.1 | + | − |
| 112 | Proteocatella sphenisci | WP_028829945.1 | + | − |
| 113 | Clostridium sp. MSTE9 | WP_009063988.1 | − | − |
| 114 | Spirochaeta africana | WP_014454236.1 | − | − |
| 115 | Deltaproteobacteria bacterium | OGQ13386.1 | − | − |
| | RIFCSPHIGHO2_02_FULL_40_11 | | | |
| 116 | Clostridiales bacterium | KKM11466.1 | − | − |
| | PH28_bin88 | | | |
| 117 | Pelosinus propionicus DSM | WP_090932308.1 | + | − |
| 118 | Propionispora vibrioides | WP_091747803.1 | − | − |
| 119 | Natronincola ferrireducens | WP_090549432.1 | − | − |
| 120 | uncultured Ruminococcus sp. | WP_112331601.1 | − | − |
| 121 | Firmicutes bacterium CAG: 41 | WP_022229858.1 | − | − |
| 122 | Tannerella sp. oral | ETK11816.1 | − | − |
| 123 | Clostridium sp. DL-VIII | WP_009171375.1 | − | − |
| 124 | Desulfobulbus japonicus | WP_028581706.1 | − | − |
| 125 | Veillonella sp. oral | WP_009353657.1 | − | − |
| 126 | Bacillus selenitireducens | WP_013174003.1 | − | − |
| 127 | Deltaproteobacteria bacterium | OGP02283.1 | − | − |
| | GWA2_38_16 | | | |
| 128 | Clostridiaceae bacterium BRH | KJS20094.1 | | |
| 129 | Clostridium cadaveris | WP_035770223.1 | − | − |
| 130 | Vibrio hangzhouensis | WP_103880502.1 | − | − |
| 131 | Halanaerobium congolense | SDI24694.1 | − | − |
| 132 | uncultured Eubacterium sp. | SCH28733.1 | − | − |
| 133 | Oscillibacter sp. CAG: 241 | CDB26907.1 | − | − |
| 134 | Clostridium sp. KLE | ERI68946.1 | + | − |
| 135 | Caldalkalibacillus thermarum | WP_007505383.1 | + | − |
| | TA2.A1 | | | |
| 136 | Budvicia aquatica | WP_029095874.1 | − | − |
| 137 | Caldalkalibacillus thermarum | WP_007505383.1 | + | − |
| | TA2.A1 | | | |
| 138 | Rhodospirillum rubrum ATCC | WP_011388669.1 | − | − |
| | 11170 | | | |
| 139 | Bacteroidetes bacterium | OFX78235.1 | − | − |
| | GWE2_39_28 | | | |
| 140 | Desulfosporosinus sp. BICA1 | KJ46946.1 | − | − |
| 141 | Clostridium uliginosum | WP_090094411.1 | − | − |
| 142 | Pseudobutyrivibrio sp. ACV-2 | WP_090301343.1 | − | − |
| 143 | Sporolituus thermophilus DSM | WP_093690468.1 | − | − |
| 144 | Eubacteriaceae bacterium | WP_087275421.1 | − | − |
| | CHKCI004 | | | |
| 145 | Blautia sp. CAG: 257 | CDA04862.1 | + | − |
| 146 | Listeria marthii FSL | EFR88049.1 | + | − |
| 147 | Desulfosporosinus sp. OT | WP_009624792.1 | − | − |
| 148 | Clostridium methoxybenzovorans | WP_024346771.1 | + | − |
| 149 | Bacillus sp. m3-13 | WP_010197697.1 | + | − |
| 150 | bacterium CG2_30_54_10 | OIP28307.1 | + | − |
| 151 | Halanaerobium sp. 4-GBenrich | ODS50009.1 | − | − |
| 152 | Candidatus Izimaplasma sp. | KFZ26741.1 | + | + |
| 153 | Desulfotomaculum guttoideum | WP_092244224.1 | − | − |
| 154 | Bacillus daliensis | WP_090843272.1 | − | − |
| 155 | Sporomusa acidovorans | WP_093796665.1 | − | − |
| 156 | Clostridium sp. C105KSO15 | WP_089994985.1 | − | − |
| 157 | Firmicutes bacterium CAG: 41 | CCZ36420.1 | + | − |
| 158 | Fusobacterium nucleatum subsp. | WP_085057258.1 | + | − |
| 159 | Thermoanaerobacterium | WP_013788835.1 | + | − |
| | xylanolyticum LX-11 | | | |
| 160 | Enterococcus pallens | WP_010758150.1 | − | − |
| 161 | Porphyromonas uenonis | WP_007364879.1 | − | − |
| 162 | Tenericutes bacterium | OHE32257.1 | − | − |
| | GWD2_38_27 | | | |
| 163 | Clostridia bacterium BRH_c25 | KUO67763.1 | − | − |
| 164 | Listeria monocytogenes | WP_012951491.1 | + | − |
| 165 | Clostridium lavalense | WP_092361844.1 | + | − |
| 166 | Acetanaerobacterium elongatum | WP_092640331.1 | + | − |
| 167 | Alkaliphilus peptidifermentans | WP_091539210.1 | + | − |
| | DSM | | | |
| 168 | Clostridium sp. C105KSO15 | WP_089983798.1 | − | − |
| 169 | Ruminococcus sp. CAG: 17 | CCY97458.1 | − | − |
| 170 | Clostridium hylemonae DSM 15053 | EEG72288.1 | − | − |
| 171 | Acetonema longum DSM 6540 | EGO64744.1 | − | − |
| 172 | Brachyspira innocens | WP_020003501.1 | − | − |
| 173 | Clostridium saccharobutylicum | WP_022747467.1 | − | − |
| 174 | Tenericutes bacterium | OHE28831.1 | − | − |
| | GWD2_38_27 | | | |
| 175 | Bacillus sp. FJAT-25547 | WP_053476394.1 | − | − |
| 176 | Clostridium populeti | WP_092561044.1 | + | − |
| 177 | Natronincola peptidivorans | WP_090442614.1 | − | − |
| 178 | Megasphaera paucivorans | WP_091652222.1 | − | − |
| 179 | Anaerobium acetethylicum | WP_091232027.1 | − | − |
| 180 | Eubacterium limosum | ALU13318.1 | − | − |
| 181 | Porphyromonas sp. CAG: 1061 | CCY08492.1 | − | − |
| 182 | Clostridium beijerinckii strain | AAD31841.1 | − | − |
| | NRRL B593 | | | |
| 183 | Clostridium sticklandii DSM 519 | WP_013360893.1 | − | − |
| 184 | Bacillus oryziterrae | WP_017754440.1 | − | − |
| 185 | Yersinia enterocolitica | WP_005157703.1 | − | − |
| 186 | Syntrophobacterales bacterium | OHE18777.1 | − | − |
| | GWC2_56_13 | | | |
| 187 | Candidatus Bacteroides | KQM08700.1 | + | − |
| | periocalifornicus | | | |
| 188 | Anaerocolumna aminovalerica | WP_091689178.1 | + | − |
| 189 | Natronincola peptidivorans | WP_090439673.1 | − | − |
| 190 | Dendrosporobacter quercicolus | WP_092070189.1 | − | − |
| 191 | uncultured Flavonifractor sp. | SCJ32847.1 | − | − |
| 192 | Geobacillus sp. Y4.1MC1 | OUM85091.1 | − | − |
| 193 | Clostridium bolteae CAG: 59 | CCX97030.1 | − | − |
| 194 | Roseburia inulinivorans A2-194 | WP_118109132.1 | − | − |

In some embodiments, the TER enzymes are as shown in Table 11.

TABLE 11
5-carboxy-2-pentenoyl-CoA reductases

| Candidate | | Accession No/ | Activity | Activity |
|---|---|---|---|---|
| # | Species | SEQ ID NO. | CP-CoA | Cr-CoA |
| 1 | Candida | XP_026596596.1/ | + | + |
| | tropicalis | SEQ ID NO: 144 | | |
| 2 | Coraliomargarita | WP_013043307.1 | − | ND |
| | akajimensis | | | |
| | DSM 45221 | | | |
| 3 | Vibrio | WP_009599222.1 | − | ND |
| | caribbenthicus | | | |
| | ATCC BAA- | | | |
| | 2122 | | | |
| 4 | Cephaloticoccus | WP_068629633.1 | + | ND |
| | primus | | | |
| 5 | Opitutaceae | WP_068772922.1 | + | ND |
| | TSB47 | | | |
| 6 | Puniceicoccaceae | PDH30576.1 | + | ND |
| | MEDG31 | | | |
| 7 | Rubritalea | WP_018969548.1 | + | ND |
| | marina | | | |
| 8 | Rubritalea | WP_105041881.1 | + | ND |
| | profundi | | | |
| 9 | Rubritalea | SHK09753.1 | + | ND |
| | squalenifa | | | |
| 10 | Verrucomicrobiae | PAW64593.1 | − | ND |
| | bacterium | | | |
| 11 | Vibrio | WP_053410267.1 | + | ND |
| | hepatarius | | | |
| 12 | Vibrio | WP_065302224.1 | − | ND |
| | natriegens | | | |
| 13 | Vibrio owensii | WP_009705827.1 | − | ND |
| | LMG 25430 | | | |
| 14 | Vibrio sp. EJY3 | WP_014231565.1 | + | ND |
| 15 | Arabidopsis | CAB75790.1 | + | ND |
| | thaliana | | | |
| 16 | Rattus | NP_058905.1 | − | ND |
| | norvegicus | | | |
| 17 | Coraliomargarita | OUU72757.1 | + | ND |
| | sp. | | | |
| 18 | Haloferula sp. | WP_035604676.1 | + | ND |
| 19 | Opitutaceae | KRP37089.1 | + | ND |
| | BACL24 | | | |
| 20 | Opitutaceae | WP_107744621.1 | + | ND |
| | EW11 | | | |
| 21 | Puniceicoccaceae | PDH31188.1 | + | ND |
| | MED-G30 | | | |
| 22 | Terrimicrobium | WP_075078790.1 | + | ND |
| | sacchariphilum | | | |
| 23 | Vibrio owensii | WP_038894812.1 | + | ND |
| | ATCC | | | |
| 24 | Drosophila | NP_610914.2 | + | + |
| | melanogaster | SEQ ID NO: 145 | | |
| 25 | Homo sapiens | NP_057095.4 | + | ND |
| 26 | Euglena gracilis | Truncated version | + | + |
| | | of Q5EU90.1 | | |

The non-naturally occurring microbial organisms are constructed using methods well known in the art as exemplified herein to exogenously express at least one nucleic acid encoding a 6-aminocaproic acid, caprolactam, hexamethylenediamine or other C5-C14 product pathway enzyme in sufficient amounts to produce 6-aminocaproic acid, caprolactam, hexamethylenediamine or other C5-C14 product. It is understood that the microbial organisms are cultured under conditions sufficient to produce 6-aminocaproic acid, caprolactam, hexamethylenediamine or other C5-C14 product. Following the teachings and guidance provided herein, the non-naturally occurring microbial organisms can achieve biosynthesis of 6-aminocaproic acid, caprolactam, hexamethylenediamine or other C5-C14 product resulting in intracellular concentrations between about 0. 1-200 mM or more. Generally, the intracellular concentration of 6-aminocaproic acid, caprolactam, hexamethylenediamine or other C5-C14 product is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring microbial organisms.

Culture conditions can include anaerobic or substantially anaerobic growth or maintenance conditions. Exemplary anaerobic conditions have been described previously and are well known in the art. Exemplary anaerobic conditions for fermentation processes are described herein and are described, for example, in U.S. Pat. No. 7,947,483, issued May 24, 2011. Any of these conditions can be employed with the non-naturally occurring microbial organisms as well as other anaerobic conditions well known in the art. Under such anaerobic conditions, the 6-aminocaproic acid, caprolactam, hexamethylenediamine or other C5-C14 product producers can synthesize 6-aminocaproic acid, caprolactam, hexamethylenediamine or other C5-C14 product intracellular concentrations of 5-10 mM or more as well as all other concentrations exemplified herein. It is understood that, even though the above description refers to intracellular concentrations, 6-aminocaproic acid, caprolactam, hexamethylenediamine or other C5-C14 product producing microbial organisms can produce 6-aminocaproic acid, caprolactam, hexamethylenediamine or other C5-C14 product intracellularly and/or secrete the product into the culture medium.

The culture conditions can include, for example, liquid culture procedures as well as fermentation and other large scale culture procedures. As described herein, particularly useful yields of the biosynthetic products can be obtained under anaerobic or substantially anaerobic culture conditions.

As described herein, one exemplary growth condition for achieving biosynthesis of 6-aminocaproic acid, caprolactam, hexamethylenediamine or other C5-C14 product includes anaerobic culture or fermentation conditions. The non-naturally occurring microbial organisms can be sustained, cultured or fermented under anaerobic or substantially anaerobic conditions. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also include growing or resting cells in liquid medium or on solid agar inside a sealed chamber maintained with an atmosphere of less than 1% oxygen. The percent of oxygen can be maintained by, for example, sparging the culture with an N2/CO2 mixture or other suitable non-oxygen gas or gases.

The culture conditions described herein can be scaled up and grown continuously for manufacturing of 6-aminocaproic acid, caprolactam, hexamethylenediamine or other C5-C14 product. Exemplary growth procedures include, for example, fed-batch fermentation and batch separation; fed-batch fermentation and continuous separation, or continuous fermentation and continuous separation. Fermentation procedures are particularly useful for the biosynthetic production of commercial quantities of 6-aminocaproic acid, caprolactam, hexamethylenediamine or other C5-C14 product. Generally, and as with non-continuous culture procedures, the continuous and/or near-continuous production of 6-aminocaproic acid, caprolactam, hexamethylenediamine or other C5-C14 product will include culturing a non-naturally occurring 6-aminocaproic acid, caprolactam, hexamethylenediamine or other C5-C14 product producing organism in sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, organisms can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of 6-aminocaproic acid, caprolactam, hexamethylenediamine or levulinic acid can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the 6-aminocaproic acid, caprolactam, hexamethylenediamine or other C5-C14 product producers for continuous production of substantial quantities of 6-aminocaproic acid, caprolactam, hexamethylenediamine or other C5-C14 product, the 6-aminocaproic acid, caprolactam, hexamethylenediamine or other C5-C14 product producers also can be, for example, simultaneously subjected to chemical synthesis procedures to convert the product to other compounds or the product can be separated from the fermentation culture and sequentially subjected to chemical conversion to convert the product to other compounds, if desired. As described herein, an intermediate in the adipate pathway utilizing 3-oxoadipate, hexa-2-enedioate, can be converted to adipate, for example, by chemical hydrogenation over a platinum catalyst.

As described herein, exemplary growth conditions for achieving biosynthesis of 6-aminocaproic acid, caprolactam, hexamethylenediamine or other C5-C14 product includes the addition of an osmoprotectant to the culturing conditions. In certain embodiments, the non-naturally occurring microbial organisms can be sustained, cultured or fermented as described above in the presence of an osmoprotectant. Briefly, an osmoprotectant means a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. For example, Escherichia coli in the presence of varying amounts of 6-aminocaproic acid is suitably grown in the presence of 2 mM glycine betaine. The amount of osmoprotectant in the culturing conditions can be, for example, no more than about 0. 1 mM, no more than about 0. 5 mM, no more than about 1. 0 mM, no more than about 1. 5 mM, no more than about 2. 0 mM, no more than about 2. 5 mM, no more than about 3. 0 mM, no more than about 5. 0 mM, no more than about 7. 0 mM, no more than about 10 mM, no more than about 50 mM, no more than about 100 mM or no more than about 500 mM.

Successfully engineering a pathway involves identifying an appropriate set of enzymes with sufficient activity and specificity. This entails identifying an appropriate set of enzymes, cloning their corresponding genes into a production host, optimizing fermentation conditions, and assaying for product formation following fermentation. To engineer a production host for the production of 6-aminocaproic acid or caprolactam, one or more exogenous DNA sequence(s) can be expressed in a host microorganism. In addition, the microorganism can have endogenous gene(s) functionally deleted. These modifications will allow the production of 6-aminocaproate or caprolactam using renewable feedstock.

In some embodiments minimizing or even eliminating the formation of the cyclic imine or caprolactam during the conversion of 6-aminocaproic acid to HMDA entails adding a functional group (for example, acetyl, succinyl) to the amine group of 6-aminocaproic acid to protect it from cyclization. This is analogous to ornithine formation from L-glutamate in Escherichia coli. Specifically, glutamate is first converted to N-acetyl-L-glutamate by N-acetylglutamate synthase. N-Acetyl-L-glutamate is then activated to N-acetylglutamyl-phosphate, which is reduced and transaminated to form N-acetyl-L-ornithine. The acetyl group is then removed from N-acetyl-L-ornithine by N-acetyl-L-ornithine deacetylase forming L-ornithine. Such a route is necessary because formation of glutamate-5-phosphate from glutamate followed by reduction to glutamate-5-semialdehyde leads to the formation of (S)-1-pyrroline-5-carboxylate, a cyclic imine formed spontaneously from glutamate-5-semialdehyde. In the case of forming HMDA from 6-aminocaproic acid, the steps can involve acetylating 6-aminocaproic acid to acetyl-6-aminocaproic acid, activating the carboxylic acid group with a CoA or phosphate group, reducing, aminating, and deacetylating.

### EXAMPLES

### Example 1. Production of 6-Aminocaproate

A highly efficient pathway for the production of adipate is achieved through genetically altering a microorganism such that similar enzymatic reactions are employed for adipate synthesis from succinyl-CoA and acetyl-CoA (see FIG. 1). Successful implementation of this entails expressing the appropriate genes, tailoring their expression, and altering culture conditions so that high acetyl-CoA, succinyl-CoA, and/or redox (for example, NADH/NAD+) ratios will drive the metabolic flux through this pathway in the direction of adipate synthesis rather than degradation. Strong parallels to butyrate formation in Clostridia (Kanehisa and Goto, Nucl. Acids Res. 28:27-30 (2000)) support that each step in the adipate synthesis pathway is thermodynamically feasible with reaction directionality governed by the concentrations of the participating metabolites. The final step, which forms adipate from adipyl-CoA, can take place either via a synthetase, phosphotransadipylase/kinase, transferase, or hydrolase mechanism. Such an engineered organism is described in U.S. Pat. No. 10,415,042, which is hereby incorporated by reference in its entirety for all purposes.

An exemplary pathway for forming caprolactam and/or 6-aminocaproic acid using adipyl-CoA as the precursor is shown in FIG. 2. The pathway involves a CoA-dependant aldehyde dehydrogenase that can reduce adipyl-CoA to adipate semialdehyde and a transaminase or 6-aminocaproate dehydrogenase that can transform this molecule into 6-aminocaproic acid. The terminal step that converts 6-aminocaproate into caprolactam can be accomplished either via an amidohydrolase or via chemical conversion (Guit and Buijs, U.S. Pat. No. 6,353,100, issued Mar. 7, 2002; Wolters et al., U.S. Pat. No. 5,700,934, issued Dec. 23, 1997; Agterberg et al., U.S. Pat. No. 6,660,857, issued Dec. 9, 2003). The maximum theoretical yield of caprolactam was calculated to be 0.8 mole per mole glucose consumed (see Table 7) assuming that the reverse adipate degradation pathway was complemented with the reaction scheme shown in FIG. 2. The pathway is favorable energetically as up to 0.78 moles of ATP are formed per mole of glucose consumed at the maximum theoretical yield of caprolactam. The ATP yield can be further improved to 1.63 moles of ATP produced per mole of glucose if phosphoenolpyruvate carboxykinase (PPCK) is assumed to function in the ATP-generating direction towards oxaloacetate formation.

An engineered organism making adipyl-CoA and then 6-aminocaproic acid from adiyl-CoA is described in U.S. Pat. No. 10,415,042, which is hereby incorporated by reference in its entirety for all purposes. This engineered microorganism made 6 aminocaproic acid.

### Example 2. 6ACA Production is Limited by Export from the Cell

A cell engineered for production of 6-aminocaproic acid (6ACA) was used (e.g., a cell engineered as in Example 1). Engineered cells were grown and intracellular and extracellular levels of 6ACA were monitored. 6ACA production in the engineered E. coli host showed an intracellular accumulation of 6ACA that plateaued, and an extracellular accumulation that stopped increased earlier in time from the intracellular plateau. Further kinetic analysis of the production rates indicated that 6ACA production is limited by transporter export of 6ACA out of the cell.

### Example 3. 6ACA Exporters Increase Production of 6ACA

The 6ACA production cell line was engineered to overexpress the transporters in Table 12 below. The transporter overexpressing cell line was then tested for production of 6ACA. The transporters ybjE (aka lysO) and yhiM produced significant increased production of 6ACA while the other nine transporters did not.

TABLE 12
Transporters Overexpressed in the 6ACA Production Cell Line

| | | | Effect of |
|---|---|---|---|
| | | | Overexpression |
| | | | on 6ACA |
| Gene | Organism | Provenance | Production |
| lysE | C. glutamicum | Well-documented lysine transporter | − |
| ybjE | E. coli | AKA lysO; lysine exporter | ++ |
| yddG | E. coli | ABC exporter with broad substrate range | No effect |
| | | including lysine | |
| yhiM | E. coli | GABA exporter | + |
| rhtA | E. coli | Threonine/homoserine exporter | − |
| argO | E. coli | Upregulation increases Arg production | No effect |
| AtGAT1 | A. thaliana | GABA exporter with 6ACA export activity | No effect |
| gabP | E. coli | 4-aminobutyrate: H + symporter/GABA permease | −− |
| tolC | E. coli | KO hypersentisizes cell to Cys | − |
| cadA | E. coli | Lysine/cadaverine antiporter | No effect |
| cadB | E. coli | Lysine/cadaverine antiporter | No effect |
| lysO | Yersina | Lysine transporter | ++ |
| | entomophaga | | |

Neither ybjE nor yhiM were known was 6ACA transporters. The A. thaliana transporter AtGAT 1 has been reported as a transporter for 6ACA but it did not increase 6ACA production. Likely its Km was high as is the Km for the endogenous 6ACA transporter in the production cell line.

The transporter ybjE was known as a lysine transporter and the transporter yhiM was known as a gamma-aminobutyric acid (GABA) transporter.

### Example 4. Deletion of 6ACA Importers Increases Production of 6-Aminocaproate

The above Examples showed that increasing extracellular 6ACA increased the overall production of 6ACA by the production cell line. Deletion of 6ACA importers (import 6ACA into the production cell line) also increased 6ACA production. The import transporters in Table 13 were deleted in the production cell and deletion of gabP and csiR increased 6ACA production while deletion of the lysP transporter did not change 6ACA production.

TABLE 13
Importers Deleted in the 6ACA Production Cell line

| | | | Effect of |
|---|---|---|---|
| | | | Overexpression on |
| Gene | Organism | Provenance | 6ACA Production |
| gabP | E. coli | 4-aminobutyrate: H + symporter/GABA permease | ++ |
| csiR | E. coli | Carbon starvation induced transporter | + |
| lysP | E. coli | Lysine symporter | No effect |

Deletion of gabP, known as a GABA permease, or csiR, an importer known to be induced by carbon starvation, increased the production of 6ACA in the production cell line. The lysine symporter, lysP, did not increase 6ACA production after its deletion.

### Example 5. Overexpression of GDH Increases Production of 6-Aminocaproate

The terminal step of 6ACA production pathway is catalyzed by a transaminase that utilizes glutamate. Overexpression of glutamate dehydrogenase (GDH) increases glutamate production that can drive the terminal transaminase step of 6ACA production. FIGS. 2A and 2B show a bar chart and graph of the increase in 6ACA production with low GDH expression, medium GDH expression and high GDH expression. Both medium and high expression of GDH increased the production of 6ACA.

### Example 6. Deletion of Mucoid Phenotype Genes Increases Production of 6-Aminocaproate

The production cell line exhibited a mucoid phenotype at times, and reducing the formation of exopolysaccharide associated with the mucoid phenotype could increase production of 6ACA. Several genes were identified as upregulated in the mucoid phenotype, and deletions of these genes were made. Deletion of the genes rcsA, rcsB, wcaF, and cpsBG made the production cell line non-mucoid, wherease deletion of the upregulated genes galF and yjb op resulted in strains that were still mucoid. Table 14 belows shows the effect of gene deletion on the mucoid phenotype and the production of 6ACA. While ΔrcsA, ΔrscB, ΔwcaF and ΔcpsBG all rendered the production cell line non-mucoid, only the deletions ΔrcsA and ΔcpsBG produced large increases in 6ACA production.

TABLE 14
The Mucoid Phenotype and Production of 6ACA

| Strain | Mucoid Phenotype | 6ACA (mM) |
|---|---|---|
| Parent | Mucoid | 11.2 |
| ΔrcsA | Non-mucoid | 41.1 |
| ΔrscB | Non-mucoid | 9.7 |
| ΔgalF | Mucoid | 11.1 |
| ΔwcaF | Non-mucoid | 12.1 |
| ΔcpsBG | Non-mucoid | 32.9 |
| Δyjb op | Mucoid | 19.1 |

The mucoid phenotype is deleterious to production properties of the 6ACA strain. The mucoid strain requires a large diameter filter, produces double layer cell pellets, the cells do not completely pellet, handling during production is more laborious, and exopolysaccharide of the mucoid phenotype takes carbon away from desired products.

The non-mucoid strain with a deletion ΔrcsA increased 6ACA production about four-fold over the parent mucoid strain. The non-mucoid strain ΔcpsBG increased 6ACA production about three-fold over the parent strain. The two other non-mucoid deletion strains had neglibile of no increase in 6ACA production compared to the parent strain.

### Example 7. 6ACA Transporters, GDH and Anti-Mucoid Deletions Increase Production of 6-Aminocaproate

A production cell line for making 6-aminocaproic acid was engineered to overexpress the ybjE (lysO) exporter and a glutamate dehydrogenase, and to disrupt rcsA to prevent the mucoid phenotype. The production cell line was also engineered with 9833T. The production of 6-aminocaproic acid by these engineered cell lines is shown in the table below:

TABLE 15
6ACA Production

| Modifications to | | | |
|---|---|---|---|
| 6ACA Production | Titer | Rate | Yield |
| Cell Line | g/L | g/L/hr | Mol/mol |
| ybjE + 9893T Integ | 30.4 | 0.63 | 0.21 |
| ybjE + ΔrcsA + | 36.1 | 0.75 | 0.28 |
| 2X9893T Integ | | | |
| ybjE + ΔrcsA + gdh + | 41.6 | 0.87 | 0.31 |
| 2X9893T Integ | | | |

Adding ybjE to an earlier version of the 6ACA production cell line increased titer from 9.6 to 15.9 (66% increase), rate from 0.13 to 0.22 (69% increase) and yield from 0.07 to 0.12 (71% increase).

Each of the changes to the 6ACA production cell line, adding an exporter (ybjE), adding gdh, and disrupting rcsA increased the production of 6ACA by the production cell line. When these changes were combined with 9893T, the overall increased in 6ACA production was about 4-fold.

### Example 8. 6ACA Transporter Activity

Putative 6ACA transporters were tested in a ΔlysO strain of E. coli that also included genes encoding the 6ACA pathway enzymes: 1) a thiolase (Thl), 2) a 3-hydoxybutryl-CoA dehydrogenase (Hbd), 3) a crotonase Crt), 4) trans-enoyl-CoA reductase (Ter), 5) aldehyde dehydrogenase (Ald), and 6) transaminase (TA). Alternatively, the putative 6ACA transporters were integrated onto the E. coli chromosome containing the pathway genes and the resulting strain was evaluated for 6ACA production.

The engineered E. coli cells were fed 5% glucose in minimal media, and after a 16-24 h incubation at 35° C., the cells were harvested, and the level of 6ACA in the supernatant was determined by standard LC/MS method or enzymatically using purified 6ACA-transaminase. For the enzymatic detection of 6ACA, the absorbance at 450 nm was measured after incubation of purified 6ACA transaminase (3 μM), 50 U/mL bovine glutamate dehydrogenase (SIGMA), 0.1 mM α-ketoglutarate, 0.1 mM NAD, 10 μM PMS (1-methoxy-5-methylphenazinium methyl sulfate and 2 mM XTT (2,3-Bis-(2-methoxy-4-nitro-5-sulfophenyl)-2H-tetrazolium-5-carboxanilide) in 0.1 M Tris-HCl, pH 7.4 buffer. The level of 6ACA in the supernatant was determined using a calibration curve that contained a known amount of 6ACA with the same components described above. For each transporter gene evaluated, the relative 6ACA export was determined from the 6ACA in the supernatant of plasmid containing the transporter gene to the 6ACA in the supernatant of a plasmid that contained no candidate gene (empty vector; negative control). The activity of a putative 6ACA transporter is scored as an amount of 6ACA in the supernatant relative to a control in which no 6ACA transporter is added ([supernatant 6ACA for putative 6ACA transporter]/[supertnatant 6ACA for no added putative 6ACA transporter]).

The results for the tested putative 6ACA transporters are shown in Table 16 below. A relative transport score of <0.80 is a minus (−), a score of 0.8-1.1 is +, and a score of 1.10-2.0 is a ++, and not detected is blank. 6ACA transporters that have a score of ++ increase the export of 6 ACA out of the cells. 6ACA transporters that reduce the amount of 6ACA exported from the cell (−and BLANK) are 6ACA importers.

TABLE 16
6ACA Exporter Activity

| UniprotKB | | |
|---|---|---|
| Accession # | 6ACA Transporter | Activity |
| P75826 | Escherichia coli | ++ |
| T4VE12 | Paraclostridium bifermentans ATCC 19299 | |
| Q8A9L0 | Bacteroides thetaiotaomicron VPI-5482 | |
| K8WCQ8 | Providencia sneebia DSM 19967 | + |
| K5Z3H8 | Parabacteroides goldsteinii CL02T12C30 | |
| F5MF26 | Shigella boydii 5216-82 | |
| A0A485CCH3 | Raoultella planticola | |
| A0A482PBI2 | Citrobacter rodentium | − |
| A0A447V4H2 | Cedecea lapagei | ++ |
| A0A359G454 | Alistipes sp. | + |
| A0A7G2M372 | Culturomica sp. | − |
| A0A331LGJ2 | Klebsiella pneumoniae | + |
| A0A168P3C4 | Klebsiella oxytoca | + |
| A0A090T2I6 | Vibrio maritimus | + |
| A0A085HLU7 | Leminorella grimontii ATCC 33999 = DSM 5078 | ++ |
| A0A085AG20 | Trabulsiella guamensis ATCC 49490 | ++ |
| A0A077MZA0 | Xenorhabdus bovienii str. puntauvense | |
| A0A7Z8FIX7 | Shigella sonnei | |
| A0A4R6EY16 | Scandinavium goeteborgense | |
| A0A854FFJ9 | Salinivibrio kushneri | |
| A0A3V8VK07 | Salmonella enterica subsp. enterica serovar Agama | |
| A0A3S6EWD1 | Yersinia entomophaga | ++ |
| A0A3R0JPG3 | Shigella dysenteriae | ++ |
| A0A3N0UJ76 | Lonsdalea populi | |
| A0A2X5EV87 | Serratia rubidaea | ++ |
| A0A2X4TF08 | Salmonella enterica subsp. arizonae | |
| A0A2X2DZ65 | Raoultella planticola | ++ |
| UniParc ID | Enterobacter cloacae complex sp. | ++ |
| UPI00045BA014 | | |
| A0A2U9SDC3 | Azospirillum ramasamyi | |
| A0A2T8R213 | Salmonella enterica subsp. enterica serovar 4, 12:i:- | |
| A0A2P2BV61 | Romboutsia hominis | |
| A0A2I8S8B5 | Citrobacter freundii complex sp. CFNIH3 | |
| A0A2C6DRS7 | Budvicia aquatica | |
| UniParc ID | Kosakonia oryzae | + |
| UPI0009A8BEF6 | | |
| A0A1S9J3T6 | Shigella boydii | |
| A0A1B9PQG6 | Aliivibrio sp. 1S175 | ++ |
| A0A0Q9CPY2 | Oerskovia sp. Root918 | ++ |
| A0A0N0DK82 | Photobacterium leiognathi subsp. mandapamensis | − |
| UniParc ID | Achromobacter sp. | + |
| UPI0000683E9E | | |
| UniParc ID | Achromobacter sp. | |
| UPI000013A380) | | |
| A0A0B6FGQ0 | Yersinia frederiksenii Y225 | ++ |
| A0A0A2XQ71 | Gallibacterium anatis | + |
| A0A0HIREZ8 | Microvirga vignae | − |
| A0A0T9KE32 | Yersinia nurmii | ++ |
| A0A1T0AXW6 | [Haemophilus] felis | + |
| A0A1Y2SR89 | Xenorhabdus beddingii | − |
| A0A2D0JQA6 | Xenorhabdus miraniensis | ++ |
| A0A2N5KTP3 | Actinobacteria bacterium | ++ |
| A0A2U3BBA9 | Vibrio sp. E4404 | ++ |
| A0A3N0UJ76 | Lonsdalea populi | − |
| A0A3S0AXG7 | Neisseriaceae bacterium | ++ |
| A0A3S6EWD1 | Yersinia entomophaga | + |
| A0A085HLU7 | Leminorella grimontii ATCC 33999 = DSM 5078 | ++ |
| A0A198FET8 | Proteus myxofaciens ATCC 19692 | ++ |
| A0A260S6R4 | Rhodococcus sp. 15-2388-1-1a | − |
| A0A0F1BDI7 | Enterobacter sichuanensis | − |
| A0A0M3EX80 | Enterobacter cloacae | + |
| A0A1C5WGH0 | uncultured Bacteroides sp. | ++ |
| A0A1I7IZI6 | Xenorhabdus koppenhoeferi | ++ |
| A0A1Q5TYK1 | Xenorhabdus eapokensis | ++ |
| A0A1Q5U965 | Xenorhabdus thuongxuanensis | ++ |
| A0A3E4Z618 | Phocaeicola plebeius | ++ |
| A0A3R9QS08 | Enterobacter huaxiensis | − |
| A0A855M686 | Enterobacter cloacae complex sp. ECNIH14 | ++ |
| A0A0B6XA16 | Xenorhabdus bovienii | ++ |
| A0A0G3CKY0 | Pragia fontium | ++ |
| A0A0H5M125 | Yersinia intermedia | ++ |
| A0A0J5FX48 | Xenorhabdus khoisanae | ++ |
| A0A2D0IU27 | Xenorhabdus ehlersii | ++ |
| A0A3A3ZES2 | Enterobacter chuandaensis | ++ |
| A0A1I3WDG2 | Mesorhizobium albiziae | + |
| A0A2N4W2H6 | Klebsiella quasipneumoniae | ++ |
| A0A4R2XZM7 | Raoultella ornithinolytica | ++ |
| A0A8B3UA18 | Enterobacter roggenkampii | + |
| A0A101K111 | Bosea sp. WAO | ++ |
| A0A443UFD0 | Enterobacter cloacae | ++ |
| A0A2Z3F3X0 | Klebsiella quasipneumoniae | ++ |
| A0A0T9TQ60 | Yersinia enterocolitica | ++ |
| A0A0T9T4V6 | Yersinia frederiksenii | ++ |
| A0A411IV75 | Enterobacter sp. 9-2 | + |
| A0A3X9TWR2 | Salmonella enterica I | ++ |
| A0A356QXL1 | Gammaproteobacteria bacterium | ++ |
| A0A2N4XFJ9 | Emticicia sp. TH156 | − |
| A0A1E4IRS8 | Variovorax sp. SCN 67-85 | |
| W3V012 | Photorhabdus khanii NC19 | ++ |
| A0A455VNE7 | Enterobacter asburiae | ++ |
| A0A0Q4NI65 | Serratia sp. Leaf51 | ++ |

| Relative 6ACA Export | Legend |
|---|---|
| <0.8 | − |
| 0.8-1.1 | + |
| 1.10-2.0 | ++ |
| >2.0 | +++ |
| Not Determined | Blank |

### Example 9: Glutamate Dehydrogenase Activity

Putative GDH candidates were cloned into an expression plasmid and transformed into E. coli. A cell suspension of E. coli with the putative GDH candidates was measured at 600 nm and the cell suspensions were normalized to an OD of 4. Cell pellets were prepared by centrifugation and the pellet was then lysed with a chemical lysis reagent containing nuclease and lysozyme for 30 minutes at room temperature. This lysate was used to measure the Gdh activity at room temperature (22-25° C.) and the assay was carried out as follows: aliquot of the crude Gdh lysate, desired concentration of a-ketoglutarate (0.5 mM), 5 mM ammonium chloride, and 0.2 mM NADH or NADPH, were mixed in 0.02 mL of 0.1 M Tris-HCl, pH 7.5 buffer. The kinetics of the reaction was monitored by NADH or NADPH oxidation using fluorescence or absorbance at 340 nm. The rate (ΔF/min) was determined using the plate reader program. Relative activity to SEQ ID NO: 10 was determined.

TABLE 17
GDH Activity

| | GenBank or | Activity | | |
|---|---|---|---|---|
| | UniprotKB | DmAbs/ | NADH | NADPH |
| GDH | Accession No. | min | ΔF/min | ΔF/min |
| Bacillus subtilis (strain 168) | P39633 | ++++ | + | + |
| Entodinium caudatum | AAF15393 | + | | |
| Halobacterium salinarium | P29051 | + | | |
| Nitrobacter agilis | GEC14679 | + | | |
| Pyrobaculum calidifontis (strain DSM | A3MUY9 | ++++ | | |
| 21063/JCM 11548/VA1) | | | | |
| Caulobacter vibrioides | OYX06180 | ++++ | | |
| Bacterioides fragilis | P94316 | ++++ | | |
| Pyrobaculum islandicum | BAA77715 | ++++ | | |
| Clostridium difficile | ARE61085 | ++++ | | |
| Mycobacterium canettii | WP_170667434.1 | ++ | | |
| Salicibibacter kimchii | A0A345BV80 | + | | |
| Mucinivorans hirudinis | A0A060RBU7 | + | | |
| Anaerococcus lactolyticus S7-1-13 | A0A095X4D3 | ++++ | | |
| Polaribacter dokdonensis DSW-5 | A0A0M9CG05 | + | | |
| Deltaproteobacteria bacterium | A0A1F9IMB6 | ++++ | | |
| RIFCSPLOWO2_02_FULL_50_16 | | | | |
| Polaribacter filamentus | A0A2S7L1V8 | ++++ | | |
| Anaerococcus prevotii DSM 20548 | C7RFH9 | +++ | | |
| Frankia inefficax | E3J4R4 | +++ | + | + |
| Moraxellaceae psychrobacter sp. | A0A229GSK5 | ++++ | | |
| DAB_AL32B | | | | |
| Flammeovirgaceae bacterium TMED32 | A0A1Z8QAP9 | ++ | | |
| Entodinium caudatum | AAF15393 | ++++ | + | ++ |
| Halobacterium salinarium | P29051 | + | | |
| Nitrobacter agilis | GEC14679 | ++ | | |
| Pyrobaculum calidifontis | A3MUY9 | ++++ | ++ | + |
| Caulobacter vibrioides | OYX06180 | ++++ | + | + |
| Bacterioides fragilis | P94316 | ++++ | +++ | + |
| Pyrobaculum islandicum | BAA77715 | ++++ | ++ | + |
| Peptoclostridium difficile | [symbol] Q18CSO | ++++ | ++ | + |
| Mycobacterium canettii | WP_170667434.1 | + | | |
| Salicibibacter kimchi | A0A345BV80 | + | | |
| Mucinivorans hirudinis | A0A060RBU7 | ++ | | |
| Anaerococcus lactolyticus S7-1-13 | A0A095X4D3 | ++++ | +++ | ++ |
| Polaribacter dokdonensis DSW-5 | A0A0M9CG05 | ++++ | ++ | ++ |
| Deltaproteobacteria bacterium | A0A1F9IMB6 | ++++ | +++ | +++ |
| RIFCSPLOWO2_02_FULL_50_16 | | | | |
| Polaribacter filamentus | A0A2S7L1V8 | ++++ | + | +++ |
| Anaerococcus prevotii DSM 20548 | C7RFH9 | ++++ | + | +++ |
| Frankia inefficax | E3J4R4 | + | | |
| Moraxellaceae psychrobacter sp. | A0A229GSK5 | ++++ | ++ | ++ |
| DAB_AL32B | | | | |
| Flammeovirgaceae bacterium TMED32 | A0A1Z8QAP9 | ++ | | |
| Anaerococcus prevotii ACS-065-V- | F0GVR9 | | | |
| Col13 | | | | |
| Sulfurimonas gotlandica GD1 | B6BKM6 | | | |
| Bdellovibrio bacteriovorus W | W5WWS1 | | +++ | ++ |
| Acidihalobacter aeolianus | A0A1D8K556 | | + | + |
| Bacillus megaterium | UniParc Id | | | |
| | UPI000BF90396 | | | |
| Bacillus sp. MRMR6 | A0A1Q9PJU5 | | | |
| Acidobacteriia bacterium | A0A317J079 | | | |
| Anaerolineae bacterium | A0A367ZGM1 | | +++ | + |
| Cyclobacterium amurskyense | A0A0H4PH76 | | | |
| Aminomonas paucivorans DSM 12260 | E3CYM8 | | | |
| Cytophagales bacterium | A0A3C2A0W4 | | + | + |
| Aneurinibacillus migulanus | A0A1G8S441 | | | |
| Candidate division TA06 bacterium | A0A0S7YDH0 | | | |
| DG_78 | | | | |
| Armatimonadetes bacterium | A0A1F3BBV3 | | | |
| RBG_19FT_COMBO_69_19 | | | | |
| Syntrophorhabdus sp. PtaB.Bin027 | A0A1V4WK45 | | +++ | + |
| Verrucomicrobia bacterium | A0A1V6GF89 | | | |
| ADurb.Bin063 | | | | |
| Terriglobus saanensis SP1PR4 | E8UZ81 | | | |
| Cyanobacterium aponinum PCC 10605 | K9Z203 | | + | ++ |
| Crocosphaera chwakensis CCY0110 | A3IL52 | | | |
| Crocosphaera watsonii WH 0005 | T2IPP1 | | | |
| Nitritalea halalkaliphila LW7 | I5C951 | | | |
| Sandaracinus amylolyticus | A0A0F6YMK4 | | + | + |
| Varibaculum cambriense | A0A134BPC5 | | ++ | + |
| Geobacter sp. DSM 9736 | A0A212PGP1 | | + | + |
| Anaerolineaceae bacterium | A0A2E1A8S9 | | + | + |
| Candidatus tagabacteria bacterium | A0A2M8ERW4 | | ++ | + |
| CG_4_9_14_0_2_um_filter_41_11 | | | | |
| Marinilabiliales bacterium | A0A2J6I8G0 | | | |
| Alkalicoccus saliphilus | A0A2T4UAG1 | | + | + |
| Bacteroidetes bacterium | A0A3M1CG83 | | +++ | +++ |
| Erwinia psidii | A0A3N6SPY0 | | + | + |
| Peptoniphilus asaccharolyticus | AAA25611 | | +++ | ++ |
| Escherichia coli K-12 MG1655 | AAA87979 | | | |

| In vitro activity - NADH (Col G) | Rate (DmAbs/min) | Description |
|---|---|---|
| + | 0: −0.03-−0.1 | no activity or very low activity |
| ++ | 1: −0.1-−0.2 | low activity |
| +++ | 2: −0.2-−0.3 | moderate activity |
| ++++ | 3: −0.2-−0.3 | high activity |

| In vitro activity - NADH or | | |
|---|---|---|
| NADPH | Rate (DF/min) | Description |
| + | <100 | low activity |
| ++ | 100-500 | moderate activity |
| +++ | >500 | high activity |

| In vitro activity - NADH or | | |
|---|---|---|
| NADPH | mmole/g/h | Description |
| + | <100 | low activity |
| ++ | >100 | high activity |
| Blank | | Not determined |

[symbol]

indicates data missing or illegible when filed

### Example 10: Reducing Agmatine and Putrescine

Arginine decarboxylase (speA, GenBank Acc #NP_417413.1, Uniprot Acc #P21170, SEQ ID NO: 49) and Agmatinase (speB, GenBank Acc #NP_417412.1, UniProt Acc #P60651, SEQ ID NO: 51) were knocked out by deletion in an E. coli strain engineered to have the HMD pathway of FIG. 1 (A B C D N O P Q R U V W).

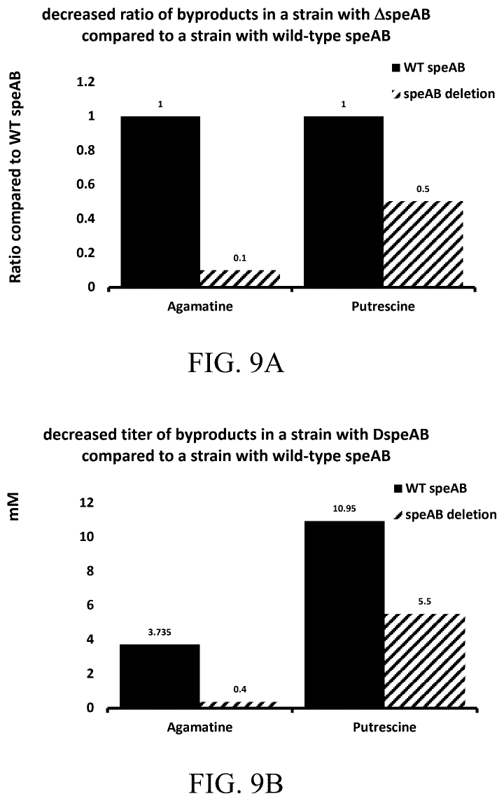

Production of 6ACA was increased in these strains as the ratio and titer of agmatine and putrescine byproducts was reduced compared to the HMD strain without the deletion. These results for the AspeAB strain are shown in FIGS. 9A and 9B.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A method for making a 6-aminocaproic acid, comprising the steps of: providing a non-naturally occurring microbial organism comprising a pathway for making a 6-aminocaproic acid and an exogenous nucleic acid encoding a transporter for the 6-aminocaproic acid, wherein the exogenous transporter exports the 6-aminocaproic acid from the cell; and culturing the non-naturally occurring microbial organism in a medium under conditions where the 6-aminocaproic acid is produced.

24. The method of claim 23, further comprising the step of transporting the 6-aminocaproic acid from the microbial organism into the medium.

25. The method of claim 24, wherein the exogenous nucleic acid encodes a transporter selected from Table 16 having a relative 6ACA export activity of greater than 1.10.

26. The method of claim 23, wherein the non-naturally occurring microbial organism further comprising a disruption of a gene encoding a transporter that imports 6-aminocaproic acid into the microbial organism.

27. The method of claim 26, wherein the gene is a gabP, or csiR.

28. The method of claim 23, wherein the non-naturally occurring microbial organism further comprising an exogenous nucleic acid encoding a glutamate dehydrogenase.

29. The method of claim 23, wherein the non-naturally occurring microbial organism further comprising a disruption of a rscA, a cpsB, a cpsG, or a cpsBG.

30. The method of claim 23, wherein the non-naturally occurring microbial organism further comprises an exogenous nucleic acid encoding a glutamate dehydrogenase, wherein at least some of the glutamate made by the glutamate dehydrogenase is used by a transaminase that produces the 6-aminocaproic acid.

31. The method of claim 30, wherein the glutamate dehydrogenase is selected from Table 17 and has an in vitro activity with NADH or NADPH of greater than 100 ΔF/min.

32. The method of claim 31, wherein the glutamate dehydrogenase is selected from Table 17 and has an in vitro activity with NADH or NADPH of 100-500 ΔF/min.

33. The method of claim 31, wherein the glutamate dehydrogenase is selected from Table 17 and has an in vitro activity with NADH or NADPH of greater than 500 ΔF/min.

34. (canceled)

35. (canceled)

36. The method of claim 23, wherein the production of the 6-aminocaproic acid by the microbial organism is increased compared to a microbial organism without the exogenous nucleic acid.

37. The method of claim 24, wherein the exogenous transporter is selected from one of the transporters in Table 16 having a relative 6ACA export activity of greater than 0.80.

38. The method of claim 28, wherein the glutamate dehydrogenase is a GdhA or a homolog thereof.

39. The method of claim 26, wherein the non-naturally occurring microbial organism further comprising an exogenous nucleic acid encoding a glutamate dehydrogenase.

40. The method of claim 39, wherein the gene is a gabP, or csiR.

41. The method of claim 39, wherein the non-naturally occurring microbial organism further comprising an exogenous nucleic acid encoding a glutamate dehydrogenase.

42. The method of claim 41, wherein the glutamate dehydrogenase is selected from Table 17 and has an in vitro activity with NADH or NADPH of greater than 100 ΔF/min.

43. The method of claim 41, wherein the glutamate dehydrogenase is selected from Table 17 and has an in vitro activity with NADH or NADPH of 100-500 ΔF/min.

44. The method of claim 41, wherein the non-naturally occurring microbial organism further comprising a disruption of a rscA, a cpsB, a cpsG, or a cpsBG.


SEQUENCE LISTING (94 sequences)

SEQ ID NO: 1 - DNA, 900 bp, Escherichia coli
atgttttctgggctgttaatcattctggttcccctgattgtgggttacctcattccgcttcgccaacaagctgcgttaaaagttattaatcagctattaagctggatggtttaccttattctcttttttatgggtatcagtctggcgtttctcgataacctcgccagtaacctgttggcgattctgcattattctgccgtcagtattaccgttattttactgtgtaatattgccgccctgatgtggctggagcgaggcctgccgtggcgcaaccaccatcagcaagaaaaactcccgtcgcgtattgcgatggcgctggagtcgctaaaactgtgcggcgtagtagtgattggttttgccattggtctaagtggactggctttcttacaacacgcgaccgaagccagtgaatacacgttaattttgctacttttcctcgttggtattcagttgcgcaataatggcatgaccttaaagcagattgtccttaatcgccggggaatgattgtcgccgtggtggtggttgtcagttcattaattggtggtttaattaacgcctttattcttgatctccccatcaataccgcgctggcaatggcctccggtttcggctggtattctctttccggtattttattgaccgaatcttttggtccggtaatcgggagcgcggcgttttttaatgatctggcccgtgaactgattgctattatgttgatccctgggctgattcgccgcagccgctctactgcactgggcttatgcggtgccacatcaatggatttcaccctgcccgttcttcaacgtactggcgggctggatatggtcccggcggcaattgttcacggttttattcttagcctgttagtgccgatcctcatcgcctttttctctgcgtag

SEQ ID NO: 2 - protein, 299 aa, Escherichia coli
MFSGLLIILVPLIVGYLIPLRQQAALKVINQLLSWMVYLILFFMGISLAFLDNLASNLLAILHYSAVSITVILLCNIAALMWLERGLPWRNHHQQEKLPSRIAMALESLKLCGVVVIGFAIGLSGLAFLQHATEASEYTLILLLFLVGIQLRNNGMTLKQIVLNRRGMIVAVVVVVSSLIGGLINAFILDLPINTALAMASGFGWYSLSGILLTESFGPVIGSAAFFNDLARELIAIMLIPGLIRRSRSTALGLCGATSMDFTLPVLQRTGGLDMVPAAIVHGFILSLLVPILIAFFSA

SEQ ID NO: 3 - DNA, 1053 bp, Escherichia coli
atgaacatatatatcgggtggcttttcaaattaatccctttgattatgggcttaatttgtatcgcgttaggtggctttgtgctggaaagttcagggcaaagcgagtatttcgtcgcgggtcatgtgctgatttctctggcggccatatgcctggcattattcactaccgcatttattatcatttcgcagctcacgcgcggcgttaatacgttttacaatacattgttccccattattggctatgcggggtcaattatcaccatgatatggggttgggcactgttagcaggcaatgatgtgatggcagacgagtttgtcgccggccatgttattttcggcgttggtatgattgccgcctgtgtatcgacggtggcagcgtcatccggtcactttctgctcattcccaaaaatgcagcggggagcaagagcgacggaacaccggtacaggcttattcttcattaatcggtaactgcctcattgccgttcccgttttactcaccctgctcggtttcatttggtctattacgctgttacgtagtgctgacataactccgcattatgtcgcgggtcacgtattgcttgggttaaccgcaatctgtgcctgtctaattggccttgttgccacaattgtccatcaaacacgtaatacgttttcaactaaagaacactggctgtggtgttattgggttatttttctcggctcaatcacggtactgcaggggatatacgtcttagtcagttccgatgcaagcgcccgactggctcccggcattattcttatttgcctcggaatgatctgttacagcatattctcaaaagtctggctactggcactggtatggagacgtacctgttcgttagccaacagaataccgatgattcccgtcttcacctgcctgttttgccttttcctggcatcgtttcttgcggaaatggcgcagaccgacatgggatattttattccttcgcgagttctggtcggtttgggagcggtatgctttacgttgttctcaatcgtttcaatattagaagcgggttctgctaaaaaatag

SEQ ID NO: 4 - protein, 350 aa, Escherichia coli
MNIYIGWLFKLIPLIMGLICIALGGFVLESSGQSEYFVAGHVLISLAAICLALFTTAFIIISQLTRGVNTFYNTLFPIIGYAGSIITMIWGWALLAGNDVMADEFVAGHVIFGVGMIAACVSTVAASSGHFLLIPKNAAGSKSDGTPVQAYSSLIGNCLIAVPVLLTLLGFIWSITLLRSADITPHYVAGHVLLGLTAICACLIGLVATIVHQTRNTFSTKEHWLWCYWVIFLGSITVLQGIYVLVSSDASARLAPGIILICLGMICYSIFSKVWLLALVWRRTCSLANRIPMIPVFTCLFCLFLASFLAEMAQTDMGYFIPSRVLVGLGAVCFTLFSIVSILEAGSAKK

SEQ ID NO: 5 - DNA, 1401 bp, Escherichia coli
atggggcaatcatcgcaaccacatgagttaggcggcgggctgaagtcacgccacgtcaccatgttgtctattgccggtgttatcggcgcaagtctgtttgtcggttccagcgtcgccatcgccgaagcgggcccggcggtattactggcctatctgttcgccgggctactggtggttatgattatgcggatgttggcggaaatggcggttgccacgcccgataccggttcgttttccacctatgccgataaagccattggtcgctgggcgggctataccatcggctggttgtactggtggttttgggtactggttatcccgctggaagccaacatcgccgccatgatcctgcactcatgggttccaggcattcccatctggttattttccctcgtcattaccctcgccttaactggcagtaacttattaagcgttaaaaactacggcgaatttgagttctggctggcgctgtgcaaagtcatcgctatcctggcctttattttccttggtgcagtcgcaattagcggtttttacccgtatgccgaagtgagcgggatctcaagattgtgggatagcggcggctttatgcccaacggtttcggtgcggtattaagcgcgatgttgatcaccatgttctcgtttatgggcgcagaaattgtcaccattgccgccgcggaatccgacacgccggaaaaacatattgtccgcgccaccaactcggttatctggcgtatttctatcttctatttatgttctatttttgtcgtcgtggcattaattccgtggaatatgcccggactaaaagccgtcggttcttatcgctcggtactggaattgctcaatattccccatgcgaaattaatcatggactgcgtgatattactttccgtaaccagttgcctgaactcggcgctgtataccgcgtcaaggatgctctactccttaagtcgtcgcggtgatgctcccgcggtaatgggtaaaatcaaccgcagtaaaaccccgtacgtggcggtgttactctccaccggcgcggcattcttaacggtggtggtgaactattacgcgcctgcgaaggtatttaaatttctgatcgacagctccggcgctatcgccctgctggtttatttagtcatcgccgtttcacagttgcggatgcgcaaaattctgcgagcagaaggaagcgaaattcgcttgcgaatgtggctttatccgtggctcacctggctagtcatcggctttattacctttgtgttggtagtgatgctattccgtccggcgcaacagttagaagtgatctccaccggcttattagcgatagggattatctgtaccgtgccaattatggctcgctggaaaaagctggtattgtggcaaaaaacacccgttcataatacgcgctga

SEQ ID NO: 6 - protein, 466 aa, Escherichia coli
MGQSSQPHELGGGLKSRHVTMLSIAGVIGASLFVGSSVAIAEAGPAVLLAYLFAGLLVVMIMRMLAEMAVATPDTGSFSTYADKAIGRWAGYTIGWLYWWFWVLVIPLEANIAAMILHSWVPGIPIWLFSLVITLALTGSNLLSVKNYGEFEFWLALCKVIAILAFIFLGAVAISGFYPYAEVSGISRLWDSGGFMPNGFGAVLSAMLITMFSFMGAEIVTIAAAESDTPEKHIVRATNSVIWRISIFYLCSIFVVVALIPWNMPGLKAVGSYRSVLELLNIPHAKLIMDCVILLSVTSCLNSALYTASRMLYSLSRRGDAPAVMGKINRSKTPYVAVLLSTGAAFLTVVVNYYAPAKVFKFLIDSSGAIALLVYLVIAVSQLRMRKILRAEGSEIRLRMWLYPWLTWLVIGFITFVLVVMLFRPAQQLEVISTGLLAIGIICTVPIMARWKKLVLWQKTPVHNTR

SEQ ID NO: 7 - DNA, 663 bp, Escherichia coli
atgaccattacgtctctggatggctatcgctggctgaagaacgatattattcgcggtaattttcaaccggatgaaaaattacgaatgagtttgctgacatcgcgttatgcacttggcgttgggccgttacgggaagctctttcgcaactggtggcggaacggctggtcacggtggtgaatcaaaaagggtatcgggtggcgtctatgtcagagcaggagctgctcgatattttcgacgcccgcgccaatatggaagcgatgttagtgagtctggcgattgcccgcggtggcgatgagtgggaggcagacgttctcgcaaaagcgcatctgctgagtaagcttgaggcctgtgacgccagcgagaaaatgcttgatgagtgggatctgcgtcatcaggcgtttcatacggcaattgtggcgggctgtggttctcactatttgctgcaaatgcgtgaacggttgtttgatctggcggcgcgttatcgatttatctggctgcggcgaacggtgctttcggtggaaatgctggaggataaacacgatcagcaccagaccctgactgcggcggtactggcgcgagataccgcgcgcgccagtgagttaatgcgccagcatttactgacgccaattcccattatccagcaggcgatggctggcaattaa

SEQ ID NO: 8 - protein, 220 aa, Escherichia coli
MTITSLDGYRWLKNDIIRGNFQPDEKLRMSLLTSRYALGVGPLREALSQLVAERLVTVVNQKGYRVASMSEQELLDIFDARANMEAMLVSLAIARGGDEWEADVLAKAHLLSKLEACDASEKMLDEWDLRHQAFHTAIVAGCGSHYLLQMRERLFDLAARYRFIWLRRTVLSVEMLEDKHDQHQTLTAAVLARDTARASELMRQHLLTPIPIIQQAMAGN

SEQ ID NO: 9 - DNA, 1344 bp, Escherichia coli
atggatcagacatattctctggagtcattcctcaaccatgtccaaaagcgcgacccgaatcaaaccgagttcgcgcaagccgttcgtgaagtaatgaccacactctggccttttcttgaacaaaatccaaaatatcgccagatgtcattactggagcgtctggttgaaccggagcgcgtgatccagtttcgcgtggtatgggttgatgatcgcaaccagatacaggtcaaccgtgcatggcgtgtgcagttcagctctgccatcggcccgtacaaaggcggtatgcgcttccatccgtcagttaacctttccattctcaaattcctcggctttgaacaaaccttcaaaaatgccctgactactctgccgatgggcggtggtaaaggcggcagcgatttcgatccgaaaggaaaaagcgaaggtgaagtgatgcgtttttgccaggcgctgatgactgaactgtatcgccacctgggcgcggataccgacgttccggcaggtgatatcggggttggtggtcgtgaagtcggctttatggcggggatgatgaaaaagctctccaacaataccgcctgcgtcttcaccggtaagggcctttcatttggcggcagtcttattcgcccggaagctaccggctacggtctggtttatttcacagaagcaatgctaaaacgccacggtatgggttttgaagggatgcgcgtttccgtttctggctccggcaacgtcgcccagtacgctatcgaaaaagcgatggaatttggtgctcgtgtgatcactgcgtcagactccagcggcactgtagttgatgaaagcggattcacgaaagagaaactggcacgtcttatcgaaatcaaagccagccgcgatggtcgagtggcagattacgccaaagaatttggtctggtctatctcgaaggccaacagccgtggtctctaccggttgatatcgccctgccttgcgccacccagaatgaactggatgttgacgccgcgcatcagcttatcgctaatggcgttaaagccgtcgccgaaggggcaaatatgccgaccaccatcgaagcgactgaactgttccagcaggcaggcgtactatttgcaccgggtaaagcggctaatgctggtggcgtcgctacatcgggcctggaaatggcacaaaacgctgcgcgcctgggctggaaagccgagaaagttgacgcacgtttgcatcacatcatgctggatatccaccatgcctgtgttgagcatggtggtgaaggtgagcaaaccaactacgtgcagggcgcgaacattgccggttttgtgaaggttgccgatgcgatgctggcgcagggtgtgatttaa

SEQ ID NO: 10 - protein, 447 aa, Escherichia coli
MDQTYSLESFLNHVQKRDPNQTEFAQAVREVMTTLWPFLEQNPKYRQMSLLERLVEPERVIQFRVVWVDDRNQIQVNRAWRVQFSSAIGPYKGGMRFHPSVNLSILKFLGFEQTFKNALTTLPMGGGKGGSDFDPKGKSEGEVMRFCQALMTELYRHLGADTDVPAGDIGVGGREVGFMAGMMKKLSNNTACVFTGKGLSFGGSLIRPEATGYGLVYFTEAMLKRHGMGFEGMRVSVSGSGNVAQYAIEKAMEFGARVITASDSSGTVVDESGFTKEKLARLIEIKASRDGRVADYAKEFGLVYLEGQQPWSLPVDIALPCATQNELDVDAAHQLIANGVKAVAEGANMPTTIEATELFQQAGVLFAPGKAANAGGVATSGLEMAQNAARLGWKAEKVDARLHHIMLDIHHACVEHGGEGEQTNYVQGANIAGFVKVADAMLAQGVI

SEQ ID NO: 11 - DNA, 624 bp, Escherichia coli
atgtcaacgattattatggatttatgtagttacacccgactaggtttaaccgggtatctgttgagtagaggggttaaaaaaagagaaatcaacgacattgaaaccgttgatgaccttgccatagcttgtgattcacagcgcccttcagtggtgtttattaatgaggactgtttcatccacgatgcttctaacagtcagcgtatcaagctcatcattaatcaacatcccaatacgttatttatcgtttttatggcaattgccaatgttcattttgatgaatatctattggtcagaaaaaatttattgatcagttctaaatcgattaaaccggaatctctcgacgatatccttggcgatattctgaaaaaagagacaacgataacctcgtttttaaatatgccgacgttatcattgagccgaaccgaatcgagtatgttgcgaatgtggatggcaggtcagggaaccattcaaatctctgaccaaatgaatatcaaagccaagaccgtttcatcgcataaaggtaatattaaacgtaagatcaaaacgcataataaacaggttatctaccatgtcgtccgactgacggataatgtgactaatggtatttttgtcaacatgcgctaa

SEQ ID NO: 12 - protein, 207 aa, Escherichia coli
MSTIIMDLCSYTRLGLTGYLLSRGVKKREINDIETVDDLAIACDSQRPSVVFINEDCFIHDASNSQRIKLIINQHPNTLFIVFMAIANVHFDEYLLVRKNLLISSKSIKPESLDDILGDILKKETTITSFLNMPTLSLSRTESSMLRMWMAGQGTIQISDQMNIKAKTVSSHKGNIKRKIKTHNKQVIYHVVRLTDNVTNGIFVNMR

SEQ ID NO: 13 - DNA, 1437 bp, Escherichia coli
atggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaa

SEQ ID NO: 14 - protein, 478 aa, Escherichia coli
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 15 - DNA, 1371 bp, Escherichia coli
atgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 16 - protein, 456 aa, Escherichia coli
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 17 - DNA, 900 bp, Escherichia coli; identical to SEQ ID NO: 1

SEQ ID NO: 18 - protein, 299 aa, Escherichia coli; identical to SEQ ID NO: 2

SEQ ID NO: 19 - DNA, 900 bp, Yersinia entomophaga
atgttttcaggcctgctcatcattttactcccgttagttgcggggtatctgatcccgttgcgccacccaagcgttctgaaactgattacccgtttattgagttggatcgtgtatgtgatcctctttttcatgggcattagcctggccttcttagacaatctgtcgtctaacttgttgagcattctgcattacgccgtcgtaagtgttgttgtcattcttctgtgtaatattgcagcgctgatgtggctggaacagaaaatgccgtggcgccaccagcatcgccaggagaaattgccttcccgcgtggctatggcgatggaatcccttcagctgtgtggtgtagttctgatcggttttctgattggtttgagcggactgagtttcctgcaacacgcgacagaggcgagtgaatataccctgatcttcctgctgttcttaattggcattcagttacgtaataatggtatgactctgcgtcagattgtgttgaaccgccgcggcatgattgtagcggtggtggttaccgcgagtagtctggcgggtggggtcatcaatgcatttatcttagatctgcccttaaagaccggccttgccatggcaagcggcttcgggtggtattctctttccgggattctcatgaccgaaagctttggtccggttatcgggagtgcagcctttttcaacgatctgagccgcgagcttctggcaattatgctgattccagggcttgtgcgtcgttcacgttcaacagcgctcgggctgtgcggcgccacgtcaatggatttcacgcttccagtgttgcagcgcagcggcggtgtagaaattgttccggctgcgattgttcatggttttgtgctgagtctgctggtcccagtccttattgctttgttctcggcttag

SEQ ID NO: 20 - protein, 298 aa, Yersinia entomophaga
MYSGLLIILLPLIIGYLIPLSRKSLIQAINRLLSWMVYVILFFMGISLAFLENLSANLLLIFQYASVCFICILSANLLALFLLEHKRPWKNTHRQEALPSRLHMALESLKLCGVVIIGFLLGLTEWPPLQFAAQGSELALIFLLFLVGIQLRNSGMTLRQIVLNRRGTIVAIVVAVSALAGGALAALFLGLPLKTGLAMASGYGWYSLSGILLTDAFGPVIGSAAFFNDLARELVAIMLIPTLVRRSRSTALGLCGATSMDFTLPVLQRSGGLEMVPAAIVHGFILSLLAPVLIALFS

SEQ ID NO: 21 - DNA, 900 bp, Shigella dysenteriae
atgttttccggcctgttaatcattttactgcctcttatcgcgggttacttgattccgttacgtcaagaaagcgcgctgcgcctgattaatcgcttcctgtctggtattgtttatctgatcctgttcttcatggggatcagcttagcatttctcgataacttgtccgctaatctgctgtccattctgcattattcggctgttactgtaaccgtgattctgctgtgcaatattgccgccctgctctggctggaacgtaccattccatggaaaaaccatcatcaccaagaaaaattaccgtcccgtatcgccatggccctggaatcattgaaactgtgtggtgtggtcgtactggggtttctgctggggttgacgggctgggcgtttttacagcatgctaccgaagcatccgagtatacgctcattttcttattgtttttaatcggtattcagctgcgtaataatggcatgacgctgaaacagattgtattaaaccgccgcggcatgattattgcggttatggttgtcgcgtcctccatggtggcaggtgttattaatgcttttattctggatctgccattaaaaacagggctggcaatggcatcaggctttggctggtactcgttgtccggcatcctgctgacagagagctttggcccagtgatcggttcggctgcgttctttaacgacctggcacgcgaactgattgctatcatgctgattccgggactcgtgcgccgtagccgcagcacggcgctgggactgtgcggcgcgactagtatggatttcactctgccggtcctgcagcgcagcggtggcttggagatggtcccggcagcaattgttcatgggtttattctctcgctgctggtcccgattttaatggcgttcttctccgcatag

SEQ ID NO: 22 - protein, 299 aa, Shigella dysenteriae
MFSGLLIILVPLIVGYLIPLRQQAALKVINQLLSWMVYLILFFMGISLAFLDNLASNLLAILHYSAVSITVILLCNIAALIWLERGLPWRNHHQQEKLPSRIAMALESLKLCGVVVIGFAIGLSGLAFLQHATEASEYTLILLLFLVGIQLRNNGMTLKQIVLNRRGMIVAVVVVASSLIGGLINAFILDLPINTALAMASGFGWYSLSGILLTESFGPVIGSAAFFNDLARELIAIMLIPGLIRRSRSTALGLCGATSMDFTLPVLQRTGGLDMVPAAIVHGFILSLLVPILIAFFSA

SEQ ID NO: 23 - DNA, 900 bp, Serratia rubidaea
atgttttcaggtctgttaattattttagtaccgctgatcgtgggatacctgatcccactgcgtcataaagccgctctgcagctgattaatcgcctgctctcctggattgtctatttaattctgtttttcatgggtatctctctcgcattcctggataaccttgcaagcaacctggtggctatctttcattatagcgccgttagtatcaccattattctgctgtgcaatatcgccgccctgctttggctggaacgtatcttaccgtggcgccaccatcatcagcaggaaaagttgccgtcgcgtatcgccatggcccttgaaagcctccagctgtgtggcgttgtagtcctcggctttgtaatcgggctgtctggtttgagcgtattgcagcacgcaaccgaagcttccgaatataccctgatttttcttctgttcctggtgggaatccagctccgtaactcgggcatgactttaaaacaaatcgtgctcaatcgccgtggtatgatggtggcggtcgtggtggtggcaagcagcttgctgggtggtgtcatcaatgcgtttattctggatctgcctctgaaaaccgctctcgcgatggcgtcgggctttggttggtatagcctgtccggtattcttctgacggaatcttttggtcccgtgatcggctccgccgcattcttcaacgacctggcccgcgagctgctggcaatcatgctgatcccgggactggttcgccgtagtcgctcgacggcactgggtctgtgtggagcgacctcaatggatttcactctgccggtactccaacgctcgggtggggttgaaattgtccctgctgccattgtgcacggttttatccttagcctgctggtcccgttgctgatggcctttttctcagcttag

SEQ ID NO: 24 - protein, 298 aa, Serratia rubidaea
MYSGLLIILVPLIAGYLIPLHSHRLIQSINRLLSRMVYVILFLMGISLAFLENLSSNLLLIFQYTAAFFLCIFLANALALYLLERKLPWRSTHKQEKLPSRLHMALESLQLCGVVLIGFLLGLTQWPWLHYATAGSEYALIFLLFLVGIQLRNSGMTLRQIIVNRRGMLVGVVVAISALAGGALAAWLLGVPVKAGLAVASGFGWYSLSAILISDAYGPVLGSTAFFNDLLRELVAIMLIPTLIRRSRSTALGLCGATSMDFTLPVLQRSGGLEIVPPAIVHGFLLSLMAPVLIALFS

SEQ ID NO: 25 - DNA, 909 bp, Yersinia frederiksenii Y225
atgtactcaggtctgttaattgtattaatgcctctgattcttggttacttcattcgtctgaataataaaacggccctggccaccgtgcactatcttctgaacatcatgatttacgtaatcttatttctgatgggtgtgtcgctggcgatgttagaaaacctgggcaacaatttgctgtccatcttgctgtatgcgatgacctttttcctttgtatcttcgccaccaatttacttgcattgttactgttagacaagcgtgatccttggattatccaggtcaataaacaagaaaagtccccctcacaactgcacatggccttcgattcgattaagctgtgcagcgcattgattctggggtttctgcttggtctgactgattggtctttgtttcactttgcgtcaccagcatccgagattgccctgatcctgttactgttgttagttggcatccagctgcgcaataatggtatgagcttaaaacaaaccctgctcaatcgccgcggcactattattgccctcgtggtcgcgatctcctccttgttaggcgggatgatcgcggcctttctgctgggtttacccaccaagacgggcctggccattgcgtccggttacggttggtattcgctgagtggcatcttgctgagtgacgcctatggcccagtgattgggtccaccgcctttttcaacgatctggcacgcgaacttgcaagtattctcctcattcctatgttaatcaaccgctaccgcagcaccgccctgggtctgacgggggctgcatctatcgatttcacactcccaatcctgcagcgctgtggtgggatcagcatcgtgccggcggcgattgtacacggctttattctgtctctgatgactccggtctttattgccttctttacccaacaggcatag

SEQ ID NO: 26 - protein, 299 aa, Yersinia frederiksenii Y225
MYSGLLIILLPLILGYLIPLSRKTLIQLINRLLSWMVYVILFFMGISLAFLENLSANLLLIFQYAGVFFLCIFCANLLALFLLERKTPWKNTHRQEVLPSRLHMALESLKLCGVVIVGFLLGLSQWEWLQFAAKGSELALIFLLFLVGIQLRNSGMTLRQIVLNRRGAIVAIVVAFSALAGGMLAAVLMDLPIKTGLAMASGFGWYSLSGILLTDSFGPVIGSAAFFNDLARELVAIMLIPTLVRSSRSSALGLCGATSMDFTLPVLQRSGGLEMVPAAIVHGFLLSLLAPILIAFFSS

SEQ ID NO: 27 - DNA, 900 bp, Yersinia frederiksenii
atgtattctgggctgctgattattctgttacctttaatcatcggctaccttattcccttgtctcgtaaaagtctcattcaacttattaatcgccttcttagttggatggtatacgtaattctgttcttcatgggtatcagtctggcgtttctcgagaacttaagcgcgaacttgttactgattttccagtacacggggattttctttctttgcatcttctgcgcaaacctgctcgcgctgttcctcctggaacgcaaaactccgtggaaaaacacgcatcgccaagaggcattgccgagccgtctgcacatggcactggaatctttaaagttgtgcggtgttgttatcgttggttttctgttaggcttaacccaatgggaatggctgcagtttgcggccaagggctcagaactggcattaatctttttactgtttctggtgggtatccagctgcgcaattctggtatgacgctgcgccagattgtgttgaaccgtcgcggcaccattgttgcgtttgtcgttgctattagcgccctggcaggtggggcgattgcggccatgctgattggcctgccggtaaaaacgggcctggctatggcaagcggtttcgggtggtatagcctgtcgggcatccttctgactgacacgtttggcccggtgattggcagtgcagcgttctttaatgacctggcacgcgaattagtggccattatgctgatcccgacattggtgcgctcatcacgttccacggcgctgggtttatgcggcgcgacttcgatggactttactctgcccgttttgcagcgctcgggcggcctggaaatggtaccagccgctatcgtgcacggattcctcctgtcgttgctggcgccgattctcatcgcgtttttcagcagttag

SEQ ID NO: 28 - protein, 299 aa, Yersinia frederiksenii
MYSGLLIILLPLIIGYLIPLSRKSLIQLINRLLSWMVYVILFFMGISLAFLENLSANLLLIFQYTGIFFLCIFCANLLALFLLERKTPWKNTHRQEALPSRLHMALESLKLCGVVIVGFLLGLTQWEWLQFAAKGSELALIFLLFLVGIQLRNSGMTLRQIVLNRRGTIVAFVVAISALAGGAIAAMLIGLPVKTGLAMASGFGWYSLSGILLTDTFGPVIGSAAFFNDLARELVAIMLIPTLVRSSRSTALGLCGATSMDFTLPVLQRSGGLEMVPAAIVHGFLLSLLAPILIAFFSS

SEQ ID NO: 29 - DNA, 900 bp, Salmonella enterica I
atgttttctgggttacttattattctcgtaccgctgatcgtggggtacttgattccgctgcgtcacaaggcggcccttcaattgattaaccgtctgttatcgtggattgtatacttgatcctcttcttcatggggatttccctcgcattcctcgataacttggcctcgaatctggttgcgatcttccattatagcgccgttagcatcaccattattctcttgtgcaacatcgcggcgctgttgtggctggagcgtattctcccctggcgccatcaccatcaacaggaaaaactgcccagtcgcatcgcgatggcgctcgagtcgctgcaactgtgtggggtagtcgtgctggggttcgttatcggcctgagtggcctgagtgtactgcagcatgcgacggaagcaagcgaatatacgttgatcttcttgttgtttttagtgggaattcagctccgcaacagcggcatgacacttaaacagattgtactcaatcgtcgcggaatgatggttgccgtggtagtcgtcgccagttcgttattaggtggcgttatcaatgcgtttatcctcgacctcccactgaagaccgcattagcgatggcaagcggctttggatggtactcactgtcaggcatcttgctgacggaggccttcggcccggttatcggctccgccgcgttctttaatgatctggcacgcgaactgctggcgattatgcttattcctggcctggtacgtcgctcacgtagcaccgcattgggtctgtgcggcgcgacgagtatggactttacactgccggtactgcagcgtagcggaggcgtagaaattgtaccggccgccattgtccatggttttatcctgtcactcctggtacctctcctgatggcgttcttttcggcatag

SEQ ID NO: 30 - protein, 299 aa, Salmonella enterica I
MFSGLLIILVPLIVGYLIPLRHKAALQLINRLLSWIVYLILFFMGISLAFLDNLASNLVAIFHYSAVSITIILLCNIAALLWLERILPWRHHHQQEKLPSRIAMALESLQLCGVVVLGFVIGLSGLSVLQHATEASEYTLIFLLFLVGIQLRNSGMTLKQIVLNRRGMMVAVVVVASSLLGGVINAFILDLPLKTALAMASGFGWYSLSGILLTEAFGPVIGSAAFFNDLARELLAIMLIPGLVRRSRSTALGLCGATSMDFTLPVLQRSGGVEIVPAAIVHGFILSLLVPLLMAFFSA

SEQ ID NO: 31 - DNA, 900 bp, Serratia sp. Leaf51
atgtactcaggactcctgattattttactgccattaatcctgggttatcttattcccgttaaaaatcacgccttgctgcacgtggttaaccaaatgctgagctggatggtctatgttattctgtttatcatgggtatttccttggcttttctggacaacctgagtaccaacctgctgctgatctttaagtacgctgcggtatgcttcttatgcatttttgttatgaattatgctgctctgtggcttctcgaacgccgccgtccgtggaaaacccagcataagcaggaaaaactgccctctcgtttgcacatggctttggaatcgctgaaattatgtggtgtagttattggtggctttgcgctgggccttactcaatggcactggctgacgtttgcttcgcaggctagcgaattagccctgctttttctgctggctctggttggcattcaattacgcaacagtagtatgaccctgcgtcagattattctgaaccgccgcggtatgatcgtcgctgtggtggtagcaatcagtgccctgatcgggggtgcactggctgccttactgttgggtttgccgctcaaaaccggattggcgctcgcatctggctacgggtggtactcactgtcgggtattgttctgacagattcgttcggtccggtaattggatccgcagcgttcttcaacgacctggcgcgtgagctgtgtgcaatcatgctgattccaactttagttcgctcctcccgctcgaccgcactcggtctgtgtggtgcgacctcaatggatttcactctgccggtattacagcgctccggtggcttggacatggtgccgcccgcggtggttcatggattcgttttatcattattgtcgccggttctcatggccttcttctctgcatag

SEQ ID NO: 32 - protein, 299 aa, Serratia sp. Leaf51
MYSGLLIILLPLILGYLIPVKNHALLHVVNQMLSWMVYVILFIMGISLAFLDNLSTNLLLIFKYAAVCFLCIFVMNYAALWLLERRRPWKTQHKQEKLPSRLHMALESLKLCGVVIGGFALGLTQWHWLTFASQASELALLFLLALVGIQLRNSSMTLRQIILNRRGMIVAVVVAISALIGGALAALLLGLPLKTGLALASGYGWYSLSGIVLTDSFGPVIGSAAFFNDLARELCAIMLIPTLVRSSRSTALGLCGATSMDFTLPVLQRSGGLDMVPPAVVHGFVLSLLSPVLMAFFSA

SEQ ID NO: 33 - DNA, 1329 bp, deltaproteobacterium bacterium RIFCSPLOWO2 02 FULL 50 16
atgaccaaaagcatcccgaaaaaagatagcagcggcacccaagaaaccaacatttttgcaaatgcactgaaacgtctggatgaagcagcacagtttgcagatattccgcctgaagcgctggaacgtctgaaacatccgaaaaaaatgattcaggtgagcattccgattcgcatggataatggtggtctgcgtatttttcagggttatcgtgttcgtcatgatcatacccgtggtcctggtaaaggtggtattcgttttcatccgaatgttaatctggatgaggttaaagcactggcactgtggatgacctgtaaatgtgcagttatgggtctgccgtttggtggtgcaaaaggtggcattaccgttaatccgaaagaactgagccgtatggaactggaacgcctgagccgtgcatatattgcccgtattgcagaatttattggtcccaaaaccgatattccggcaccggatgtttataccaatccgatgattatgggttggatgatgatggaatatagcaaaattgcccaagaacagagtccggcagttattaccggcaaaccgattccgttaggtggtagcctgggtcgtgaagatgcaaccggtcgtggtggttattattgtattaaagagctggaagagaagaaacgttggaaaccgagcaaaattcgtgttgccattcaaggttttggtaatgtgggtcagaatattgcacgtctgctgcatgccgatggctataaaatcgttgcagttagcgatagccaaggcggtatctataaagaagaaggttttgatattccgagcctgatccagaataaaaacgcaacccgtcgtctgaaagcaatctattgtaccggtagcgtttgtgaaaccattcaggttaaaagcctgagcaataaagatctgctggaacttgatgtggatattctgattccggcagcactggaaaatcagattaccgagaaaaatgccagcaaaatcaaagccagcaacattctggaactggcaaatggtccgattaccaccgaagcagataaaatcctgaacaaaaaaggtgttctggtggttccggatatcctggccaatggtggtggtgttaccgttagctattttgaatgggttcagaataaccagggctattattggagcgaagaagaagttcacgcaaaactgcataccttcatgatccgcgaatttaacaatgtgtatcatctgatgaccaaccgcaaaataaacatgcgtaccgcagcatatgttcatgccctgaatcgtattggtgaagcagttgaagcacgtggcaccagcaaatactttctgggtaatccgtag

SEQ ID NO: 34 - protein, 442 aa, deltaproteobacterium bacterium RIFCSPLOWO2 02 FULL 50 16
MTKSIPKKDSSGTQETNIFANALKRLDEAAQFADIPPEALERLKHPKKMIQVSIPIRMDNGGLRIFQGYRVRHDHTRGPGKGGIRFHPNVNLDEVKALALWMTCKCAVMGLPFGGAKGGITVNPKELSRMELERLSRAYIARIAEFIGPKTDIPAPDVYTNPMIMGWMMMEYSKIAQEQSPAVITGKPIPLGGSLGREDATGRGGYYCIKELEEKKRWKPSKIRVAIQGFGNVGQNIARLLHADGYKIVAVSDSQGGIYKEEGFDIPSLIQNKNATRRLKAIYCTGSVCETIQVKSLSNKDLLELDVDILIPAALENQITEKNASKIKASNILELANGPITTEADKILNKKGVLVVPDILANGGGVTVSYFEWVQNNQGYYWSEEEVHAKLHTFMIREFNNVYHLMTNRKINMRTAAYVHALNRIGEAVEARGTSKYFLGNP

SEQ ID NO: 35 - DNA, 1272 bp, Anaerococcus prevotii DSM 20548
atgagcgaaaaactggatattctggaagcaacccgtgatcgttacaaaaaagcatgcgataaactgaaactggacagcgcagtttatgaaattctgaaagaaccggaacgtctgattgaagttgcaattccgattaaaatggatgacggcaaaaccaaagtgtttaaagcatttcgtagcgcacatagcagcgcactgggtccgagcaaaggtggtattcgttttgataaaaatgtgacccgtgaagaagttatggcactgagcatgatgatgagcattaaagttgcactgctgggtctgccgttaggtggtggtaaaggtggtgttattgttgatccgaaagaactgagcgaacgtgaaattgaaagcctgagccgtggttttgttcgtgcaaccaataactatctgggtagccgtattgatattccggcaccggatgttaataccaacgccaaaattatgggctatttcatcgacgaatacattgcactgaatcagggtcgtgaagatattgcaacctttaccggcaaaccgctggcattaggtggtagctttgcacgtgatcaggcaaccggttttggtgttgcactggcagttaaatatgcctatgaacgtaaagatgaaggcctgaaaggcaaaacctttatttgtcaaggttttggcaatgtgggttacttcgcagccaaatatatgagcgaatttggtgcacgcctgattgcagttaatgcaagcgatcgtaaagcaccgagcggtagcagcgccattattaacgaagatggtctggatgttgaagaactgcgcaaactgaaagaagatggtagtagcgttctggattatgcagatagccgtaaaattagcaacgaagaattttttgccctggacaccgatattattctgccgtgtgcactggaaaatgtgatcaccgaaaaaatcgccaaaaccatcaaagccaaagtgattagcgaaggtgcaaatggtccgaccacacctggtggtgcacaggttctggaagataaaggtgttgttctgattccggatattatggcaaatagcggtggtgttctggttagccattatgaatggattcagaatcagatcggctactatttcgattacgacaaggtgaaagataaagaagagggcgatctgctgcgtgtttttgaacgtatttttgatatggccgaggaagagaacgttgatctgcgtgaagcaagctttatggttgcaattaaaagcatggcagaagccctgaaatacaaaggtcgttattag

SEQ ID NO: 36 - protein, 423 aa, Anaerococcus prevotii DSM 20548
MSEKLDILEATRDRYKKACDKLKLDSAVYEILKEPERLIEVAIPIKMDDGKTKVFKAFRSAHSSALGPSKGGIRFDKNVTREEVMALSMMMSIKVALLGLPLGGGKGGVIVDPKELSEREIESLSRGFVRATNNYLGSRIDIPAPDVNTNAKIMGYFIDEYIALNQGREDIATFTGKPLALGGSFARDQATGFGVALAVKYAYERKDEGLKGKTFICQGFGNVGYFAAKYMSEFGARLIAVNASDRKAPSGSSAIINEDGLDVEELRKLKEDGSSVLDYADSRKISNEEFFALDTDIILPCALENVITEKIAKTIKAKVISEGANGPTTPGGAQVLEDKGVVLIPDIMANSGGVLVSHYEWIQNQIGYYFDYDKVKDKEEGDLLRVFERIFDMAEEENVDLREASFMVAIKSMAEALKYKGRY

SEQ ID NO: 37 - DNA, 1266 bp, Bacteroidetes bacterium
atgagcaccccgaccaccaccgcacaagaaagcatgtatgaagcagttctggcacgtctgaatgttgcagcacagattattaacctgccggaaccgattaccgaagttctgcgtcatccgcagaaagaaatcaaagttagcctgccgattgtgatggataacggtaagattaaagtgtttgaaggttatcgtgttgtgcatagcacccatctgggtccgagcaaaggtggtattcgttatgcaatggatgtgaacgaagatgaagttcgtgcactggcagcatggatgacctttaaatgtgcagttgccgatctgccgtatggtggtgcaaaaggtggcattaaatgtgatccgaaacaaatgagcgaaggtgaactggaacgtctgagccgtgcatatgccgttgcaatgaaagatgttattggcgtgaataaagatattccggcaccggatatgggcaccagcgcacgtgaaatggcatggattctggatgaatataacaaaattaccggtgaagatgcaccgggtgttattaccggcaaaccggttggtttaggtggtagcctgggtcgtgaagcagcaaccggtcgtggtgttatgattaatagtctgcaggcactgagcaaactgaacattcagccgaaacaggcaaccgcagcagttcaaggttttggtaatgttggtagccatgcagcacgtctgctggcagaacagggtattaaagttattgcaattagtgatgcaagcggtggctactataatgaaaaaggcattaacattcaggatgccctgaattatgcgggtaaaaacaataaaagcctgaacggttatccgaacgccaccaaaattacaaatgaacagctgctgaccctgagcgttgatctgctggttcctgcagcactgcagaatgtgattacctatgaaattgcccagaacgtgaagtgcaaaattatcgttgaaggtgccaatggtccgacactgccggaagcagatcaggttctgaaagataaaggtattattgtggtgccggatattctggcaaatagcggtggtgttaccgttagctattttgaatgggtgcagaacaaaatgggctattattggaccgaagatgaggtgaatcagaaacacgatgagaaaatgaaaatcgcctttgagaaagtgtggaacaacgcacagaaatatcagaccagcatgcgtattgcagcatatattaccgcactggaaaaaattcagctgggcattaaaatgaaaggccatttttag

SEQ ID NO: 38 - protein, 421 aa, Bacteroidetes bacterium
MSTPTTTAQESMYEAVLARLNVAAQIINLPEPITEVLRHPQKEIKVSLPIVMDNGKIKVFEGYRVVHSTHLGPSKGGIRYAMDVNEDEVRALAAWMTFKCAVADLPYGGAKGGIKCDPKQMSEGELERLSRAYAVAMKDVIGVNKDIPAPDMGTSAREMAWILDEYNKITGEDAPGVITGKPVGLGGSLGREAATGRGVMINSLQALSKLNIQPKQATAAVQGFGNVGSHAARLLAEQGIKVIAISDASGGYYNEKGINIQDALNYAGKNNKSLNGYPNATKITNEQLLTLSVDLLVPAALQNVITYEIAQNVKCKIIVEGANGPTLPEADQVLKDKGIIVVPDILANSGGVTVSYFEWVQNKMGYYWTEDEVNQKHDEKMKIAFEKVWNNAQKYQTSMRIAAYITALEKIQLGIKMKGHF

SEQ ID NO: 39 - DNA, 1269 bp, Anaerococcus lactolyticus S7-1-13
atgaccaccgcatatgaacagtttattaacctgcagaagaaactgaaagaggttagcgatctggcaggttttagcgaaagcttttataacattattcgtgagccggaacgcatcattgaagttaatattccggtgaaaatggatgatggtacaacccgtacctttcgtgcatttcgtagcgcacatagcagcgcactgggtccgagcaaaggtggtgttcgttatgatgaaagcgttacctatgaagaggttaaagttctgagcaccctgatgagcctgaaagttgcactgctgggtctgccgttaggtggtggtaaaggtggtattgttgttgatccgaaaaaactgagcgaacgtgaactggaagcactgagccgtggttttgttcgtgccattaataactatattggtccgcgtattgatgttccggcaccggatgttaataccaatggcaaaattatgggctatttcaccgatgaatacattgccctgaatggtaatcgtcatgatattgcaacctttaccggcaaaagcaccgatatgggtggtagcctgggtcgtaccgaagcaaccggttttggtgtttatctgaccatcaaaaagtactacgagaaaattggcaaaagcctggatggtgcgacctttgcactgcaaggttttggtaatgttggtagctttgcagcacgttttctgacccaggatggtgcaaaactgattgcactgaatagcaaagacaaaagccagaaaagcggtagcagcgccatttatgatccgaatggtctggatgttgagaaactggaaaaagcacgtgaagaaaccggttcagccctgaatattgaagccaaaaaaatcctgaacgaagagttttttgcgatcccgtgtgatattctgattccggcagcaatggaaaacgtgattgatgaaaccaatgcgggtaacattaaagcaagcctggttgttgaagcagcaaatggtccggttaccaaagccggtgaagcaattctgaatgaaaaaaacattccgatcatcccggatattctggcaaatagcggtggtgtgctggttagccattatgaatggattcagaatatgaccggtagctattgggatgaagatgaagttatgaccaaacaagagaaggatatgagcaaagccattggtgaagtttttgcaaccgcagagaaatacaaagtgaattttcgtgaagccagctttattctgagcctgagccgtattgaaaaagccctgaaactgcgtggtcgtatttag

SEQ ID NO: 40 - protein, 422 aa, Anaerococcus lactolyticus S7-1-13
MTTAYEQFINLQKKLKEVSDLAGFSESFYNIIREPERIIEVNIPVKMDDGTTRTFRAFRSAHSSALGPSKGGVRYDESVTYEEVKVLSTLMSLKVALLGLPLGGGKGGIVVDPKKLSERELEALSRGFVRAINNYIGPRIDVPAPDVNTNGKIMGYFTDEYIALNGNRHDIATFTGKSTDMGGSLGRTEATGFGVYLTIKKYYEKIGKSLDGATFALQGFGNVGSFAARFLTQDGAKLIALNSKDKSQKSGSSAIYDPNGLDVEKLEKAREETGSALNIEAKKILNEEFFAIPCDILIPAAMENVIDETNAGNIKASLVVEAANGPVTKAGEAILNEKNIPIIPDILANSGGVLVSHYEWIQNMTGSYWDEDEVMTKQEKDMSKAIGEVFATAEKYKVNFREASFILSLSRIEKALKLRGRI

SEQ ID NO: 41 - DNA, 1344 bp, Polarobacter filamentus
atggaactgaaaatcaaagaattcatggaaatggtcaagacccgcaataatcatgaaccggaatttctgcaggcagttcaagaagttgcagaaaccgttattccgtatattgccaaccacgatatctacaacggtaaaaacattctgctgcgtatggtggaaccggaacgtctgattagctttcgtgttagctgggttgatgataatggtgaaattcaggtgaatcgcggttatcgcattcagatgaatagcgcaattggtccgtataaaggtggtctgcgttttcatccgaccgttaatgcaagcattctgaaatttctggcctttgaacaggtgtttaaaaacagcctgaccacactgccgatgggtggtggtaaaggtggcagtgattttgatccgaaaggtaaaagcgaaaacgagattatgcgtttttgccacagctttatgagcgaactgtatcgtcatattggccataacaccgatgttccggcaggcgatattggtgttggtagccgtgaaattggttttatgtttggcatgtacaaaaaactgaacaacagctttaccggtgttctgaccggcaaaggtgcaagctggggtggtagcctgattcgtccggaagcaaccggttatggcaccgtttattttgcacagaatatgctgctgcgcaaagaagatagctttgcgggtaaaaaagttgtgattagcggtagcggtaatgttgcacagtatgcagcagaaaaagcaattgaactgggtgcaaccgttctgaccctgagcgatagcggtggttatatcctggatgaagaaggcattaataccgagaaactgaagcacatcatgtacatcaaaaacgaaaaacgtggtcgcatcagcgagtataccgaaaaatatccgaatgccaaatttgttgccggtggtcgtccgtggtcagttaaatgtgatattgcactgccgtgtgcaacccagaatgaactgaatggtgatgaagcaaaacagctgattaaaaacggttgtatgtgtgttagcgaaggtgcaaatatgccgagcacaccggaagccattcatgagtttcagaacgcaaaaattctgttcgcaccgggtaaagcaagcaatgcaggcggtgttgcaaccagcggtctggaaatgagccagaatagcctgcgtctgagctggtcacgtaaagaagtggacgataaactgaaagatatcatggaagatatccacgatagctgcgttgaatatggcgaaaatgaagatggcaccatcgattatatcaagggtgccaatattgccggttttgttaaagttgccgatgcaatgctggcacagggtgttgtttag

SEQ ID NO: 42 - protein, 447 aa, Polarobacter filamentus
MELKIKEFMEMVKTRNNHEPEFLQAVQEVAETVIPYIANHDIYNGKNILLRMVEPERLISFRVSWVDDNGEIQVNRGYRIQMNSAIGPYKGGLRFHPTVNASILKFLAFEQVFKNSLTTLPMGGGKGGSDFDPKGKSENEIMRFCHSFMSELYRHIGHNTDVPAGDIGVGSREIGFMFGMYKKLNNSFTGVLTGKGASWGGSLIRPEATGYGTVYFAQNMLLRKEDSFAGKKVVISGSGNVAQYAAEKAIELGATVLTLSDSGGYILDEEGINTEKLKHIMYIKNEKRGRISEYTEKYPNAKFVAGGRPWSVKCDIALPCATQNELNGDEAKQLIKNGCMCVSEGANMPSTPEAIHEFQNAKILFAPGKASNAGGVATSGLEMSQNSLRLSWSRKEVDDKLKDIMEDIHDSCVEYGENEDGTIDYIKGANIAGFVKVADAMLAQGVV

SEQ ID NO: 43 - DNA, 1269 bp, Bdellovibrio bacteriovorus W
atggcaaatggtcatgatctgtatgaaggtccgctgtttaataacgcactgaaagcactggaacatgccggtaaaatcattaattgcaatccgaacgtgatggaaaaactgcgtcgtccgcgtcgtagcattaccgttagcattccggttcgtatggatgatttttccgttaaagtgtttaccggttatcgtgttcagtattgtgcaaccctgggtccgtataaaggtggtattcgttttcatcctgatgttggtctggcagaagttgcaggcctggcagcactgatgacctttaaaaacagcctgctgggtctgccgttaggtggtgcaaaaggtggtgttaccgttgatccgagcaaactgagcgcaaccgaaaaacaaattctgacccgtcgttataccagcgaaattggtccgtttatgggtccgcagaaagatattccggcaccggatgttggcaccgatgcacagaccatggcatggatgatggatacctatagccaagaaaatggtggttttacccagaccggtgttgttaccggcaaaccggttgaaattggtggtagcgttggtcgtaatcatgcaacaggtctgggtgttatttatgttaccgaacatgcctttcacaaaaaaggcctggattttcagggtgcacgtattgcaattcaaggttttggtaatgttggtgcaagcgcagcacattttgcacatctgcgtggtgccaaagttattgccattagtgatgttagcggtgcctattttaacggtgatggtattgatattccgagcgcaattcgtcatatcaagacctataaatacctggaaggtttccagggtgcagaactgattagccatccggaactgtttgaactgccgtgtgaagcactgattccgtgtgcactggaaaatcagattacagaacataacgcacatagcatccaggccaaaatgattgttgaaggtgccaatggtccgctgagccatgaagcaaccaaaattctgagcgaaaaaggcgtttttattgtgccggatctgattgcgaatggtggtggtgtgattgttagctattttgaatgggttcaagacaccatgagctatttttgggaagaaccggaagtgcatgaacgtctgaaaagcattattctgaaagccttcgaaaccagctatgcctttagcattgacaaaaaaaccgatatgcgtgatgcagcaatggcagttgccattcagcgtctggaaaaagcaatgctgctgcgtggtatgtgtccgtag

SEQ ID NO: 44 - protein, 422 aa, Bdellovibrio bacteriovorus W
MANGHDLYEGPLFNNALKALEHAGKIINCNPNVMEKLRRPRRSITVSIPVRMDDFSVKVFTGYRVQYCATLGPYKGGIRFHPDVGLAEVAGLAALMTFKNSLLGLPLGGAKGGVTVDPSKLSATEKQILTRRYTSEIGPFMGPQKDIPAPDVGTDAQTMAWMMDTYSQENGGFTQTGVVTGKPVEIGGSVGRNHATGLGVIYVTEHAFHKKGLDFQGARIAIQGFGNVGASAAHFAHLRGAKVIAISDVSGAYFNGDGIDIPSAIRHIKTYKYLEGFQGAELISHPELFELPCEALIPCALENQITEHNAHSIQAKMIVEGANGPLSHEATKILSEKGVFIVPDLIANGGGVIVSYFEWVQDTMSYFWEEPEVHERLKSIILKAFETSYAFSIDKKTDMRDAAMAVAIQRLEKAMLLRGMCP

SEQ ID NO: 45 - DNA, 1338 bp, Bacteroides fragilis
atgaacatcgagaaaattatgagcagcctggaagcaaaacatccgggtgaaagcgaatatctgcaggcagttaaagaagttctgctgagcattgaggatatctataatcagcatccggaattcgagaaaagcaaaattatcgaacgtctggttgaaccggatcgcatttttacctttcgtgttacctgggttgatgataaaggtgaagttcagaccaatctgggttatcgtgttcagtttaataacgccattggtccgtataaaggtggtattcgttttcatgcaagcgtgaatctgagcatcctgaaatttctgggttttgagcagacctttaaaaacgcactgaccacactgccgatgggtggtggtaaaggtggcagcgattttagtccgcgtggtaaatcagatgcagaaattatgcgtttttgccaggcatttatgctggaactgtggcgtcatctgggtcctgatatggatgttccggcaggcgatattggtgttggtggtcgtgaagttggttatatgtttggcatgtacaaaaaactgacccgtgagtttaccggtacatttaccggcaaaggtctggaatttggtggtagcctgattcgtccggaagcaaccggttttggtggtctgtattttgttaaccagatgctgcagaccaaaggcattgatattaaaggcaaaaccgttgccattagcggttttggcaatgttgcatggggtgcagcaaccaaagcaaccgaactgggtgcaaaagttgttaccattagtggtccggatggctatatctatgatccgaatggtattagcggtgagaaaatcgattatatgttagaactgcgtgccagcggtaatgatattgttgcaccgtatgcagatgaatttccgggtagcacctttgttgcaggtaaacgtccgtgggaagttaaagcagatattgcactgccgtgtgcaacccagaatgaactgaatggtgaagatgccaaaaacctgattgataacaatgttctgtgcgtgggcgaaattagcaatatgggttgtacaccggaagccattgacctgtttattgaacacaaaaccatgtacgcaccgggtaaagcagttaatgccggtggtgttgcaaccagcggtctggaaatgagccagaatgcaatgcatctgagctggtcagcagccgaagttgatgaaaaactgcatagcattatgcatggtattcatgcccagtgtgttaaatatggcaccgaacctgatggctacatcaattatgttaaaggtgcaaacattgccggttttatgaaagttgcacatgcaatgatgggtcagggcattatttag

SEQ ID NO: 46 - protein, 445 aa, Bacteroides fragilis
MNIEKIMSSLEAKHPGESEYLQAVKEVLLSIEDIYNQHPEFEKSKIIERLVEPDRIFTFRVTWVDDKGEVQTNLGYRVQFNNAIGPYKGGIRFHASVNLSILKFLGFEQTFKNALTTLPMGGGKGGSDFSPRGKSDAEIMRFCQAFMLELWRHLGPDMDVPAGDIGVGGREVGYMFGMYKKLTREFTGTFTGKGLEFGGSLIRPEATGFGGLYFVNQMLQTKGIDIKGKTVAISGFGNVAWGAATKATELGAKVVTISGPDGYIYDPNGISGEKIDYMLELRASGNDIVAPYADEFPGSTFVAGKRPWEVKADIALPCATQNELNGEDAKNLIDNNVLCVGEISNMGCTPEAIDLFIEHKTMYAPGKAVNAGGVATSGLEMSQNAMHLSWSAAEVDEKLHSIMHGIHAQCVKYGTEPDGYINYVKGANIAGFMKVAHAMMGQGII

SEQ ID NO: 47 - DNA, 1266 bp, Peptoniphilus asaccharolyticus
atgacagatacacttaatccgttagtagcggcacaagaaaaagtaagaatagcatgcgaaaaattaggatgcgatccagcagtatatgaactattaaaagaaccacaaagagtaattgaaatctcaattccagtaaaaatggatgatggtacagttaaagtgttcaaaggatggagaagtgctcactcaagcgctgtaggtccatcaaaaggtggagttagattccatccaaatgtaaacatggatgaagttaaagctctttctctatggatgacattcaaaggtggagcactaggcttaccatacggcggaggaaaaggtggaatctgcgtagatccagcagaactatcagaaagagaattagaacaattatcaagaggatgggtaagaggtctttataaatatcttggagacagaatcgatatcccagcaccagacgtaaacactaacggacaaatcatgagctggttcgttgatgaatatgtaaaattaaacggcgaaagaatggacatcggaactttcacaggaaagccagtagcatttggcggaagtgaaggaagaaacgaagcaactggattcggagtagctgtagtagttagagaatctgctaagagattcggaatcaaaatggaagatgctaaaatagctgttcaaggtttcggaaacgtaggtactttcactgttaagaacattgaaagacaaggcggaaaagtttgtgctatcgctgaatgggatagaaacgaaggaaactatgctctatacaatgaaaatggaatcgacttcaaagaattattagcttacaaagaagctaacaaaactcttatcggattcccaggagcagaaagaattactgatgaagaattctggacaaaagaatatgatatcatagtaccagcagcattagaaaatgtaatcacaggcgaaagagctaaaacaataaacgctaaattagtttgtgaagcagctaatggtcctacaactccagaaggagacaaagtattaactgaaagaggaatcaacttaacaccagatatcttaactaactcaggtggagttctagtatcttactatgaatgggtacaaaatcaatatggatactactggacagaagcagaagtagaagaaaaacaagaagcagacatgatgaaagctatcaaaggcgtattcgcagttgctgatgaatacaatgtaactctaagagaagctgtttacatgtatgcaatcaaatcaatagatgtagctatgaaattaagaggatggtattag

SEQ ID NO: 48 - protein, 421 aa, Peptoniphilus asaccharolyticus
MTDTLNPLVAAQEKVRIACEKLGCDPAVYELLKEPQRVIEISIPVKMDDGTVKVFKGWRSAHSSAVGPSKGGVRFHPNVNMDEVKALSLWMTFKGGALGLPYGGGKGGICVDPAELSERELEQLSRGWVRGLYKYLGDRIDIPAPDVNTNGQIMSWFVDEYVKLNGERMDIGTFTGKPVAFGGSEGRNEATGFGVAVVVRESAKRFGIKMEDAKIAVQGFGNVGTFTVKNIERQGGKVCAIAEWDRNEGNYALYNENGIDFKELLAYKEANKTLIGFPGAERITDEEFWTKEYDIIVPAALENVITGERAKTINAKLVCEAANGPTTPEGDKVLTERGINLTPDILTNSGGVLVSYYEWVQNQYGYYWTEAEVEEKQEADMMKAIKGVFAVADEYNVTLREAVYMYAIKSIDVAMKLRGWY

SEQ ID NO: 49 - DNA, 1977 bp, Escherichia coli
atgtctgacgacatgtctatgggtttgccttcgtcagcgggcgaacacggtgtactacgctccatgcaggaggttgcaatgagctcccaggaagccagcaagatgctgcgtacttacaatattgcctggtggggcaataactactatgacgttaacgagctgggccacattagcgtgtgcccggacccggacgtcccggaagctcgcgtcgatctcgcgcagttagtgaaaactcgtgaagcacagggccagcgtctgcctgcactgttctgtttcccacagatcctgcagcaccgtttgcgttccattaacgccgcgttcaaacgtgcgagggaatcctacggctataacggcgattacttccttgtttatccgatcaaagttaaccagcaccgccgcgtgattgagtccctgattcattcgggcgaaccgctgggtctggaagccggttccaaagccgagttgatggcagtactggcacatgctggcatgacccgtagcgtcatcgtctgcaacggttataaagaccgcgaatatatccgcctggcattaattggcgagaagatggggcacaaggtctatctggtcattgagaagatgtcagaaatcgccattgtgctggatgaagcagaacgtctgaatgtcgttcctcgtctgggcgtgcgtgcacgtctggcttcgcagggttcgggtaaatggcagtcctccggcggggaaaaatcgaagttcggcctggctgcgactcaggtactgcaactggttgaaaccctgcgtgaagccgggcgtctcgacagcctgcaactactgcacttccacctcggttcgcagatggcgaatattcgcgatatcgcgacaggcgttcgtgaatccgcgcgtttctatgtggaactgcacaagctgggcgtcaatattcagtgcttcgacgtcggcggcggtctgggcgtggattatgaaggtactcgttcgcagtccgactgttcggtgaactacggcctcaatgaatacgccaacaacattatctgggcgattggcgatgcgtgtgaagaaaacggtctgccgcatccgacggtaatcaccgaatcgggtcgtgcggtgactgcgcatcacaccgtgctggtgtctaatatcatcggcgtggaacgtaacgaatacacggtgccgaccgcgcctgcagaagatgcgccgcgcgcgctgcaaagcatgtgggaaacctggcaggagatgcacgaaccgggaactcgccgttctctgcgtgaatggttacacgacagtcagatggatctgcacgacattcatatcggctactcttccggcatctttagcctgcaagaacgtgcatgggctgagcagctttatttgagcatgtgccatgaagtgcaaaagcagctggatccgcaaaaccgtgctcatcgtccgattatcgacgagctgcaggaacgtatggcggacaaaatgtacgtcaacttctcgctgttccagtcgatgccggacgcatgggggatcgaccagttgttcccggttctgccgctggaagggctggatcaagtgccggaacgtcgcgctgtgctgctggatattacctgtgactctgacggtgctatcgaccactatattgatggtgacggtattgccacgacaatgccaatgccggagtacgatccagagaatccgccgatgctcggtttctttatggtcggcgcatatcaggagatcctcggcaacatgcacaacctgttcggtgataccgaagcggttgacgtgttcgtcttccctgacggtagcgtagaagtagaactgtctgacgaaggcgataccgtggcggacatgctgcaatatgtacagctcgatccgaaaacgctgttaacccagttccgcgatcaagtgaagaaaaccgatcttgatgctgaactgcaacaacagttccttgaagagttcgaggcaggtttgtacggttatacttatcttgaagatgagtaa

SEQ ID NO: 50 - protein, 658 aa, Escherichia coli
MSDDMSMGLPSSAGEHGVLRSMQEVAMSSQEASKMLRTYNIAWWGNNYYDVNELGHISVCPDPDVPEARVDLAQLVKTREAQGQRLPALFCFPQILQHRLRSINAAFKRARESYGYNGDYFLVYPIKVNQHRRVIESLIHSGEPLGLEAGSKAELMAVLAHAGMTRSVIVCNGYKDREYIRLALIGEKMGHKVYLVIEKMSEIAIVLDEAERLNVVPRLGVRARLASQGSGKWQSSGGEKSKFGLAATQVLQLVETLREAGRLDSLQLLHFHLGSQMANIRDIATGVRESARFYVELHKLGVNIQCFDVGGGLGVDYEGTRSQSDCSVNYGLNEYANNIIWAIGDACEENGLPHPTVITESGRAVTAHHTVLVSNIIGVERNEYTVPTAPAEDAPRALQSMWETWQEMHEPGTRRSLREWLHDSQMDLHDIHIGYSSGIFSLQERAWAEQLYLSMCHEVQKQLDPQNRAHRPIIDELQERMADKMYVNFSLFQSMPDAWGIDQLFPVLPLEGLDQVPERRAVLLDITCDSDGAIDHYIDGDGIATTMPMPEYDPENPPMLGFFMVGAYQEILGNMHNLFGDTEAVDVFVFPDGSVEVELSDEGDTVADMLQYVQLDPKTLLTQFRDQVKKTDLDAELQQQFLEEFEAGLYGYTYLEDE

SEQ ID NO: 51 - DNA, 921 bp, Escherichia coli
atgagcaccttaggtcatcaatacgataactcactggtttccaatgcctttggttttttacgcctgccgatgaacttccagccgtatgacagcgatgcagactgggtgattactggcgtgccgttcgatatggccacttctggtcgtgcgggtggtcgccacggtccggcagcgatccgtcaggtttcgacgaatctggcctgggaacacaaccgcttcccgtggaatttcgacatgcgtgagcgtctgaacgtcgtggactgcggcgatctggtatatgcctttggcgatgcccgtgagatgagcgaaaagctgcaggcgcacgccgagaagctgctggctgccggtaagcgtatgctctctttcggtggtgaccactttgttacgctgccgctgctgcgtgctcatgcgaagcatttcggcaaaatggcgctggtacactttgacgcccacaccgatacctatgcgaacggttgtgaatttgaccacggcactatgttctataccgcgccgaaagaaggtctgatcgacccgaatcattccgtgcagattggtattcgtaccgagtttgataaagacaacggctttaccgtgctggacgcctgccaggtgaacgatcgcagcgtggatgacgttatcgcccaagtgaaacagattgtgggtgatatgccggtttacctgacttttgatatcgactgcctggatcctgcttttgcaccaggcaccggtacgccagtgattggcggcctgacctccgatcgcgctattaaactggtacgcggcctgaaagatctcaacattgttgggatggacgtagtggaagtggctccggcatacgatcagtcggaaatcactgctctggcagcggcaacgctggcgctggaaatgctgtatattcaggcggcgaaaaagggcgagtaa

SEQ ID NO: 52 - protein, 306 aa, Escherichia coli
MSTLGHQYDNSLVSNAFGFLRLPMNFQPYDSDADWVITGVPFDMATSGRAGGRHGPAAIRQVSTNLAWEHNRFPWNFDMRERLNVVDCGDLVYAFGDAREMSEKLQAHAEKLLAAGKRMLSFGGDHFVTLPLLRAHAKHFGKMALVHFDAHTDTYANGCEFDHGTMFYTAPKEGLIDPNHSVQIGIRTEFDKDNGFTVLDACQVNDRSVDDVIAQVKQIVGDMPVYLTFDIDCLDPAFAPGTGTPVIGGLTSDRAIKLVRGLKDLNIVGMDVVEVAPAYDQSEITALAAATLALEMLYIQAAKKGE

SEQ ID NO: 53 - DNA, 1287 bp, Syntrophorhabdus sp. PtaB Bin027
atggaaaaaagcagctataacccgtttaccaatgcacaggcacagtttgataaagtggcaagctttattggtctggatgaagcaacctgtgatctgctgcgtcagccgctgaaagaacatcatgtgctgattccggtttgtatggatgatggcaaaatgaaaatcttcaaaggttttcgcgtgctgcataatgatgcacgtggtccggcaaaaggtggtattcgttttcatccgcaagaaaccgcagataccgttcgtgcactggcaatgtggatgacctggaaatgtgcagttgttgatattccgttaggtggtggtaaaggtggtgttatttgtgatccgcataatctgagcgaacgtgaacaagaacgtctgtgtcgtggttgggttcgtcagctgagcaaagaaatgggtccgtatattgatgcaccggcaccggatgttatgaccaccgcaaaacatatgctgtggatgctggatgaatttgaaaccattcatggtggtcgttatccgggttttattaccggcaaaccggttggtatgggtggtagcaccggtcgtaccgaagccaccggttttggtgcaatttatgttctgcgtgaagcactgaaagacctgcagattaaactggaagaaaccagcctgagcgttcaaggttttggtaatgttgcacagtatgcagccaaaatgtttaccgaattaggtggcaaagttgttgcagttgcatgttgggataatgaggataaagtgagctatacctaccagaaaaaagaaggcctgagcatgaatatgctgctggaaattaccgatagcttcggctgcattaataagaaaaaagccgcagcagcaggctgtaaaattctgcctggtgaagaatggattgaacaggatgtggatattctgatgcctgcagcactggaaaatcagattaccgcaaaaaacgtcaacaagatcagcaaacaggttaaggttctgctggaaggtgcaaatggtccgaccaccaccgatgcagatgatattatcaaagaacgcggtatttttctgctgccggattttctggcaaatgccggtggtgtgacctgtagctattttgaacaggttcagagcaacctgaactactattgggaaaaagaagaggtgctggaaaaactggataccaaaatgaccagcgcatatcgtgcagtttatgacctggcaaaaaagaaaaacctgtatatgcgtgatgcagcctatgtgattgcaattaatcgtgttgcccaggcagttaaaatgcgtggctgggcatag

SEQ ID NO: 54 - protein, 428 aa, Syntrophorhabdus sp. PtaB Bin027
MEKSSYNPFTNAQAQFDKVASFIGLDEATCDLLRQPLKEHHVLIPVCMDDGKMKIFKGFRVLHNDARGPAKGGIRFHPQETADTVRALAMWMTWKCAVVDIPLGGGKGGVICDPHNLSEREQERLCRGWVRQLSKEMGPYIDAPAPDVMTTAKHMLWMLDEFETIHGGRYPGFITGKPVGMGGSTGRTEATGFGAIYVLREALKDLQIKLEETSLSVQGFGNVAQYAAKMFTELGGKVVAVACWDNEDKVSYTYQKKEGLSMNMLLEITDSFGCINKKKAAAAGCKILPGEEWIEQDVDILMPAALENQITAKNVNKISKQVKVLLEGANGPTTTDADDIIKERGIFLLPDFLANAGGVTCSYFEQVQSNLNYYWEKEEVLEKLDTKMTSAYRAVYDLAKKKNLYMRDAAYVIAINRVAQAVKMRGWA

SEQ ID NO: 55 - DNA, 1338 bp, Anaerolineae bacterium
atgccggtttatcatttcagcgaaaccttttatcgcgaagagatgattgttatggccgagcagattaatgcatttcagatggcacaggcacagtttgatggtgttgcaaaactgctgcgtctggatccggcaattgcagaaattctgcgttggccgatgcgtgaatttgcatttcgtattccggttcgtatggatgatggtagcattcgtgtttttcagggttttcgtgttcagcataatgatgcacgtggtccgaataaaggtggtattcgttttcatccgagcgaaaccctggataccgttcgtgcactggcaacctggatgacctggaaatgtgcagttgcagatattccgttaggtggtggtaaaggtggcgttgttgttgatcctgcaacactgagcaccggtgaaaaagaacgtctgtgtcgtggttgggttcaagcaatgtggcgtaatctgggtccgcgtattgatgttccggcaccggatgttggcaccacaccgcagatgatgggttggatgatggatgaatatagcaaactggttggtcagtatacaccgggtgtttttaccggcaaaccgcttggtggtggtggcagcgaaggtcgtaccgaagcaaccggttatggtgtgatttattgtgttcgtgaagccatgaaacacctgaaaatggatccgaccaaatgtgttgcagcaattcaaggttttggtaatgttgcacagtatgcagccattggctttgtggaaattcttggtggcaaagttgcatgtgttagctgttgggatcgtaaagataaaaccagctatacctatagccacaaggatggtattaatccgcgttttctgctgagcattaccgatcagtatggcaccattgataaagacaaagcagttgccgcaggttataccgttgaagatggtggtgcatggattagcaaagaagccgacgttctgattccggcagcactggaaggtcaggttaatgcagaaaccgtgaaaaaaatgagcagccgtgttcgtattgttgccgaaggtgcaaatggtccgtgtacaccggaagcagatgaatttttcaaacagaacaacatctttaacatcccggactttctgtgtaatgccggtggtgttaccaccagttattttgaaagcgttcagaacgacatgaacttttactggaccaaagaagaagtgctgcagaaactggataccaaactgacccaggcatttcatgcagttctggaaatgagcgaaaaagaaaaagtgtatatgcgtgatgcagcctatatggttgcaattgatcgtgttgttaaagcaatgcagctgcgtggctgggtttag

SEQ ID NO: 56 - protein, 445 aa, Anaerolineae bacterium
MPVYHFSETFYREEMIVMAEQINAFQMAQAQFDGVAKLLRLDPAIAEILRWPMREFAFRIPVRMDDGSIRVFQGFRVQHNDARGPNKGGIRFHPSETLDTVRALATWMTWKCAVADIPLGGGKGGVVVDPATLSTGEKERLCRGWVQAMWRNLGPRIDVPAPDVGTTPQMMGWMMDEYSKLVGQYTPGVFTGKPLGGGGSEGRTEATGYGVIYCVREAMKHLKMDPTKCVAAIQGFGNVAQYAAIGFVEILGGKVACVSCWDRKDKTSYTYSHKDGINPRFLLSITDQYGTIDKDKAVAAGYTVEDGGAWISKEADVLIPAALEGQVNAETVKKMSSRVRIVAEGANGPCTPEADEFFKQNNIFNIPDFLCNAGGVTTSYFESVQNDMNFYWTKEEVLQKLDTKLTQAFHAVLEMSEKEKVYMRDAAYMVAIDRVVKAMQLRGWV

SEQ ID NO: 57 - DNA, 909 bp, Cedecea lapagei
atgttctctggtatgctgttaatctttagcccgctgatcattggctacctgatccccatcaaacaagtgcgctatctgaacgcggtcaataccttgactatgcgcctggtgatggtgatcctggtcttaatgggcctgtccctggcgggtctggataaccttggtcagcatatcagtacgatcctgtatttcgcggtcaccttctttatttgcattggcgcgaccaacctgttagctctgccagtgctggataagctgtttccgaccgaaactcacggtcaccaacataaactgaccttaagcaccatgatgtccgaatccggcaaattaatcgccgcggtggccgtggggttggtggtgggtctgctgctgaaccacgacctctcgtgggtggacacggcctcggaaggcatcctcctgctgctgttactgctgatcgggattcagctgcgcaacagcggaatgaccctcaagcaaatcattctgaataaaagcggcttaatgattgctgcgctcgtggtggctacaagcctgcctgggggtctgcttgcggcttggtggctggacattccctaccatcatggattagctatggcatcgggttttggttggtattcacttgccgggattctgatgggcgacggtcttggacccgtttatggtggggcctcgtttatcttggagctggcccgtgagctgatggccattatgattatccctattttaattcgtcgttatcctctcaccgcgattggctatggcggggcgaccgccatggattttacgctgcccattattcagagcaccggcggcatccgctgcgtacccattgcaattgttagcggctttattctctccctcctggttcctgtcttgatgttatttttcttaagcctgggtaccaattag

SEQ ID NO: 58 - protein, 299 aa, Cedecea lapagei
MLSGLLIILVPLIAGYLIPLKQPPLLRLINRLLSWIVYVILFLMGISLAFLDNLASNLLTILHYSVISVVVILLCNVAGLLLLERSLPWRHQHRQEKLPSRLAMALESLQLCGVVALGFLLGLTGFEVLKYATEASEYTLIFLLLLVGIQLRNSGMTFRQIVLNRRGMIVAIVVVVSSLIAGVINAFLLGLPIKTGMAMASGFGWYSLSGILMTESYGPVIGSATFFNDLVRELLAIMLIPALVRRSRSTALGLCGATSMDFTLPVLQRSGGVEIVPAAIVHGFILSLLTPLLIAFFAS

SEQ ID NO: 59 - DNA, 900 bp, Leminorella grimontii ATCC33999 DSM 5078
atgttcagtggcttattaatcatcttagtccctctgattgttggttatctgatcccgttacgtcagcaggcagctctgaaagtcatcaatcaactgctgtcctggatggtgtatttgatcttatttttcatggggatttccttagcttttctcgataacctggctagcaatcttctcgcaattctccattattcggcagtgtcaattaccgttatccttctgtgtaatattgctgctttgatttggctcgaacgtgggctcccgtggcgtaatcatcatcagcaagaaaaattgcccagtcgtatcgcgatggcgctggaatcgctgaaattgtgtggtgtagtcgtgatcggttttgcgattggtctgagcggtttagcgtttttgcagcatgccactgaggcgagcgaatataccctgattctgctgctgttcctggtggggattcagctgcgtaataacggtatgaccctgaaacagatcgtgctcaatcgccgcggtatgatcgtggcggtagtggtggttgcatcctcactgatcggaggcctgatcaatgcatttatccttgacctcccaatcaacaccgcactggcgatggcttcgggctttggctggtacagtctgagcggaatcttactcacagaaagtttcggtccggtgattggctctgccgcattctttaatgatctggcgcgcgagctcatcgcaatcatgctcatcccaggcctcatccgccgctcccgtagcacggccttggggctgtgcggggcaacctcgatggattttacgttaccggtcctgcagcgtaccggtggtctggatatggttccggccgcgattgttcacggctttattctcagtcttctggtgccgattttaattgcattctttagtgcgtag

SEQ ID NO: 60 - protein, 300 aa, Leminorella grimontii ATCC33999 DSM 5078
MYSGLAIVLLPLIAGYLIPLKSVDALRTIHRTLGAMVYIILFFMGISLAFLDNLAANLQTILLYSVTFFIAIFGTNLIALLLLDKRWPWRLPRLKKEAGSRLHMILESLQLCGVVAGGFLIGLTHYPWLKHASTASEVALVILLLLIGIQLRNSGMTIKEITLNKRGAAIALTVALSAAVGGSLAALLLGLPINVGIAMSSGYGWYSLSGILLTDAFGPVLGSAAFFNDLARELAAILVIPSLIQRRQSAALGVCGATSMDFTLPVLQRSGGVEIVPAAIVHGFILSLLAPVFMALFSAA

SEQ ID NO: 61 - DNA, 903 bp, Trabulsiella guamensis ATCC 49490
atgtacagtggattgctgattattttattgccgttggtgttaggttacctgattccggtacgtaacaaaacgttgctgcagggggtaaatcgcctcctcagttggatggtgtacgtcattcttttcctgatgggcatcagtttggcattcttggagaatctggggtcgaacctgatggctgtactgcaatacgccgctgtttcgaccgtgtgcattattgcagctaacttgctggccctgcgcttactggaacgccgtaacgcttggaaagctaccacggttagtcacgatgcgcttccctctcgccttaagatgatgttagaaagtttacagctgtgcggcgtagtcctggtgggctttcttcttggtctgtcgcagtggccgccgctgaaattcgctgcatcaggcggggagtatgcactcttagtcctgctgtgccttgtcggcattcagttgcgcaactccgggatgacattgcgtcagatcgtgctgaatcgccgcggcaccatcatcgcagtggtggttgcggtgtctgctttgtgtggtggtgctctggcatcattgctgctcgatttacctctgaaaacaggcctggcgatggcaagcggttatggttggtattcgctgtcaggcattctgatgaccaacgcgtatggtcctgtgattgggtctgcagcgttcttcaacgacttagcacgtgaactcattgcaattacccttattccaagcctgattcgttcacgtcgtgcatcggctctgggactctgcggcgccacttctatggacttcaccttaccggtcttacaacgctcaggtggggtcgacatggtcccgccggcaatcgttcacggcttcattctgagtttgctcagtcctgtgctcattacgttgtttgttgcatag

SEQ ID NO: 62 - protein, 299 aa, Trabulsiella guamensis ATCC 49490
MFSGLLIILLPLIAGYLIPLRHSATLKLINRLLSWIVYVILFFMGISLAFLDNLTSNLLAILHYSAVSIVIILLCNIVALLWLERVLPWRHQHQQEKLPSRIAMALESLKLCGVVVLGFLLGLTGLAFLQHATEASEYTLIFLLFLVGVQLRNNGMTLRQIILNRRGMIVAVIVVISSLVGGAMNAFILDLPLKTSLAMASGYGWYSLSGILLTEAFGPVIGSAAFFNDLARELIAIMLIPGLVHRSRSTALGLSGATSMDFTLPVLQRSGGIEIVPAAIVHGFILSLLVPLLMAFFSA

SEQ ID NO: 63 - DNA, 900 bp, Raoultella planticola
atgttttctggcctgttaatcattttggtgcctctgatcgtcggttatttgattccgttgcgtcatcgcgccgcgctcaaattaattaaccgtctgttgtcatggatcgtatatcttatcttatttttcatgggcatcagtctggcttttctggataacttagccagcaacctgctggcgattttacattacagcgcagtgtctgttacgattatcttgctctgcaatattgctgcgctgttttggctggagcgtagtctgccgtggcgtcacaaccataaacaggagaaactgccatcgcgtatcgcgatggcgctggaatccttacagctgtgcggcgtcgtggttgtaggcttcctgttggggctgaccggactgcctattttgcaacatgcgaccgaagccagtgaatataccctcatttttctgctgtttctggtaggcattcagctgcgcaactcggggatgaccctgaagcaaattgtgctgaaccgtcgcgggatgatcgttgcagttgttgttgttgcaagtagtatgctggcgggtgttatcaacgctctgttactgggcttgccgctgaagaccgcgctggctatggcgagcggcttcgggtggtatagcctttctggtattctgcttaccgagtcatttggcccggttattggaagcgcggcgttcttcaacgacttagcacgcgagttaatcgccattatgcttatcccaggcatggttggacgtagccgctctactgcccttggactgtgcggtgcgactagcatggactttaccttaccggtcctgcagcgttcgggaggacttgaaatggtacctgctgcgatcgtgcatggtttcattttgagcttgttagtgccgctgcttatggcgtttttctccgcttag

SEQ ID NO: 64 - protein, 299 aa, Raoultella planticola
MFSGLLIILLPLVAGYLIPLRHPSVLKLITRLLSWIVYVILFFMGISLAFLDNLSSNLLSILHYAVVSVVVILLCNIAALMWLEQKMPWRHQHRQEKLPSRVAMAMESLQLCGVVLIGFLIGLSGLSFLQHATEASEYTLIFLLFLIGIQLRNNGMTLRQIVLNRRGMIVAVVVTASSLAGGVINAFILDLPLKTGLAMASGFGWYSLSGILMTESFGPVIGSAAFFNDLSRELLAIMLIPGLVRRSRSTALGLCGATSMDFTLPVLQRSGGVEIVPAAIVHGFVLSLLVPVLIALFSA

SEQ ID NO: 65 - DNA, 903 bp, Enterobacter cloacae complex sp.
atgtattctgggctgttaatcattttattaccacttttcttcggttacctgatccccattaagcagcctagtctgttacacaatattcatcgtctgttatcgcttatggtctatgttattctcttctttatgggcatcagcttggcctttctcgataatctgtcggctaatttagtgacgatttttctgtatgcaggtgtctttttcatctgcatcatcagcgccaaccttgcagccctggcattacttgatcgtcagtggccatggaaaaatgcccatcagcaggagaaactgccttcgcgcctcggtatggctctcgaaagtcttcaactttgcggcatcgttgctattggctttgtactgggtctgactcacattagttggctgatttatgccacgcaggctagcgaattcgcactgatttttctgctgttattagtgggcatccagctgcgcaactcagggatgagcctgcgtgaaattacgctgaatcgccgcggcatgatcatcgcccttgtggtggtgttttctgccatggttgggggtgcgattgcggcttggctgttgggactgcctattcgcaccggcttggcgatgagcagcgcatatggctggtatagcttatctggtattattttgaccgatagcctgggccctgtgctgggttcagccgcgtttttcaatgatctggcacgtgaattagtagcgatcatgttaatcccggcccttgtgcgccgtaacgtgagcagcgcagtaggcatctgcggcgcgactagcatggattttactctgccggttttgcagcgttctggtggactggccatcgtaccggcagccattgtccacggctttattcttagcctgtttgcgccggtactgttggcgttcttctcggctccgtag

SEQ ID NO: 66 - protein, 299 aa, Enterobacter cloacae complex sp.
MFSGLLIILLPLIAGYLIPLRQESALRLINRFLSGIVYLILFFMGISLAFLDNLSANLLSILHYSAVTVTVILLCNIAALLWLERTIPWKNHHHQEKLPSRIAMALESLKLCGVVVLGFLLGLTGWAFLQHATEASEYTLIFLLFLIGIQLRNNGMTLKQIVLNRRGMIIAVMVVASSMVAGVINAFILDLPLKTGLAMASGFGWYSLSGILLTESFGPVIGSAAFFNDLARELIAIMLIPGLVRRSRSTALGLCGATSMDFTLPVLQRSGGLEMVPAAIVHGFILSLLVPILMAFFSA

SEQ ID NO: 67 - DNA, 900 bp, Aliivibrio sp. 15175
atgtttagtggtttattaattatcctggtaccgttaatcgtgggttatttaattccgctgcgccagcaggcagcgctgaaagttattaatcagctgctgagttggatggtgtatctgatcttattctttatgggcatttctctggcgtttctggacaatctggcttcaaaccttcttgcgatcctgcactacagcgcggtttccattacggtgattctcctgtgcaacatcgccgccctgatgtggctggaacgcggcttgccgtggcgtaaccatcatcaacaagaaaaactccctagccgcatcgcaatggcgctggaatcactgaaactctgcggcgttgtcgttattggctttgcgatcggcctttctggccttgcgtttttgcagcacgccaccgaagcctcagaatataccctgattctgttactgttcttggttggcattcaacttcgtaataacggcatgacgctgaagcagatcgtcttaaaccgccgtggaatgatcgtggcggtggtagttgtggtcagttcgctgattgggggtctgatcaacgcctttatcttggatcttccaattaacaccgcgctggcgatggcgagcgggttcggttggtattcattaagcggcattttattaacggaatctttcggtccagtgattggcagcgcggccttctttaatgatctggcgcgtgaactgatcgcgattatgctgatccccgggctgatccgccgctcgcgcagcacggcattgggcctttgtggtgctacgagcatggattttaccctgccggtgttgcaacgcacgggcggcttagacatggtcccggcagcgatcgtgcacggctttatcctcagcctgttggtcccaattcttatcgcgttctttagtgcgtag

SEQ ID NO: 68 - protein, 299 aa, Aliivibrio sp. 15175
MLSGMLFVFLPLLFGYLIPLHSQKPLQHINAATSHLVTIILALMGLSLAALDNLAQNLNQIVTITAVFFISISVCNLSVLPIIDKLFPLNSEGNKNTLPLFKMILESAKLLLAVAAGLAFGLLSGIDLTWVESASEIILLALLFLIGIQLRNSGLSLRQILLNKHGIIIAGVILSTSLIGGLIAALFLDIPLNHGLAMASGFGWYSLAGILMGDALGPVYGGAAFLNELMRELVALLLIPTLIHRYPVTSIGYAGATAMDFTLPVIQNCGGIRCVPIAIVSGFILSLLVPFLMLFFVSL

SEQ ID NO: 69 - DNA, 900 bp, Oerskovia sp. Root918
atgtatagcggcctgctcattatcctgctcccgttaattctgggttacctcatcccactgtctcgcaaaaccctcatccagctgatcaatcgtttactgagctggatggtttatgttattctgtttttcatgggcattagcctggccttcctggaaaatctgtcagcgaatctgctgttgattttccagtatgctggggttttctttttatgcatcttttgtgctaaccttctggcattgttcctgttggaacgtaagaccccatggaaaaacacgcatcgtcaggaagttctgccctctcgtttgcatatggctttggagtcactgaaattatgtggcgttgtcattgtaggtttcttattaggtttaagccaatgggaatggttacaatttgctgccaaagggagtgaactggcacttatctttttacttttccttgttggaattcagctgcgcaatagcggtatgaccctgcgtcagattgtccttaatcgtcgtggggctatcgtcgcgatcgttgtggcgttctcagcgctggcagggggtatgctggcggcggtactgatggacctgccaattaaaacgggactcgccatggcctctggttttggctggtattctctgagcggcattctcctgaccgatagcttcggccctgtaattgggtcggccgcctttttcaatgatttagcgcgcgagctggtggcaatcatgctgattccgacgctcgtgcgttcatcccgctcatcggccctgggcctgtgcggcgcgactagcatggacttcaccctgcccgtactgcagcgtagtggcgggctcgaaatggttcctgccgcaattgtgcatggattcctgctttcgctgttagctccgattttgattgcattctttagttcgtag

SEQ ID NO: 70 - protein, 347 aa, Oerskovia sp. Root918
MNPSISVLFRAIPLAMGAVCLAFGLYVLSGGDDANHFVAGHVNVALTAICIALFTTAATIIRQLVHRYGRVWEIVLPVLGYAVAIATMIWGITIIGRGDEPQFIVAGHVMLGIGFIAGCVSTVATASTKFVLIQKSAALPVGGGAPDGAYSRGAGTVLIAIPALFAVVGLIVAVTLYARGGNAALVAGNVMVGLSLVCSALVALVASIVRQVRNEFGDAERYRWTWWVVAMGTINVALGLVVLFSSDDPSRLAPGTVLIGLGLICFSILSKVLLLALVWRQVFALANRIPIIPVATALACLFFAAFLFEATMTEPGFFVGAHVLVGLGAVCFTLFSIVSILEAGTSK

SEQ ID NO: 71 - DNA, 960 bp, Actinobacteria bacterium
atggatacaaaatcaaacgttacagataaatctagtaaaacacagggccttctgtttggcttcctcggtgtgctcgcgttcagctttacggtgccgcttacacgcgtcgctgtcgctgacttagatccgaccttcgtggggcttggacgtgccttagtggctgcagccctggccgctctgctgctggcatttacgcgtcagcgtttaccagagcgccgccattggccgcgcctggttattgtgggctccggggtaattgtcggctttcccttatttaccagcctggccctgcgtgaactgccagcagcggatgccgcagttattgtgggccttctgccagcagcgactgtggttatggcagtgttacgtgcgggggaacgtccgagccgcgcgttttggggcgcttgtgcgtttgggctgcttgcagttctcttgttcgcagcttcccaaggagctggtcttgtcccggatcgtggacacctgctcgttctgctggccgtcgccctttgttcgttgggctacgcagaaggcggtgccttggcgcaagaaatcggcggctggcgtgttatctcctggtcgttggttgtgtttgcaccgtttctggcgccatttgtcgcctggagcgtcttgggcccaggtggcgcggggcttaccgtggcgggcccagcggcatgggcatgttttgcctatgttagtgtcgtcagtatgtttttgggctttttcgcatggtaccgtgggctcgcggagggaggcgttgcgcgcgtcggacaagtgcaactgagccaaccggttttaacgctgttatggtctgcgctcctgctgggagaacgtgtaggtccgttcacggtgttgtgcgcagtacttgtgctcgcatccgtggcagttggccagcgcactcgtgtgaataaaggtggtgcccgcaccgggaaagccgcagcgaaaaccggccgtgaaggttag

SEQ ID NO: 72 - protein, 319 aa, Actinobacteria bacterium
MDTKSNVTDKSSKTQGLLFGFLGVLAFSFTVPLTRVAVADLDPTFVGLGRALVAAALAALLLAFTRQRLPERRHWPRLVIVGSGVIVGFPLFTSLALRELPAADAAVIVGLLPAATVVMAVLRAGERPSRAFWGACAFGLLAVLLFAASQGAGLVPDRGHLLVLLAVALCSLGYAEGGALAQEIGGWRVISWSLVVFAPFLAPFVAWSVLGPGGAGLTVAGPAAWACFAYVSVVSMFLGFFAWYRGLAEGGVARVGQVQLSQPVLTLLWSALLLGERVGPFTVLCAVLVLASVAVGQRTRVNKGGARTGKAAAKTGREG

SEQ ID NO: 73 - DNA, 909 bp, Vibrio sp. E4404
atgttttcggggatgctgttcatctttagtccactgatcgcaggttactttgtgagcctgagcaatgaatctttactgaacaagattaaccgctatactagcaatatcatttatattattttgagcttaatggggctgagtttagcagcgttagataatttgggctcgaacttgcagagtatcttgatttacacgggctctttcttcgtaatcatcggactgtgcaacctgtcagcgttaccattcatcgataaaattctgccgctcaaaacagaacaaaccgggaagcccatcccgctctcgatgatgcttaaagacagcattacgttgatcatggtcgtgggcagcggtctgatcattgggattttacttccgattaatctttcctgggttgatacggcgattgagtggacgctgtttttactgcttctgtttattggcattcagctgcgtaattctggtctgacgctgcgtcaaatcttgctgaacaaacatggcatgattatcagcgttgtgatcattctgtcgagctgggccggtggcattatcgccgcgttcattctggatctgccaatctatcagtccctggcaatgagtagcgggtttgggtggtatagcctgagcggtatcttaatgggtgatgcgtatggcccagtgtttggcggcgcgagttttatgttagaattattacgcgaactggtggccctggtgtcgattcctctgatggttcgcaatcgcccatgtacggccattggttatgcgggcgccaccgctatggattttacccttccagtgatccagtccagtggcggtgtgcgctgcgtgccagtggctattgtgagtggtttcattctgtccctggtggtgccgttcttcatgctgttctttgtgagcctgggtcctgtttag

SEQ ID NO: 74 - protein, 302 aa, Vibrio sp. E4404
MFSGMLFIFSPLIAGYFVSLSNESLLNKINRYTSNIIYIILSLMGLSLAALDNLGSNLQSILIYTGSFFVIIGLCNLSALPFIDKILPLKTEQTGKPIPLSMMLKDSITLIMVVGSGLIIGILLPINLSWVDTAIEWTLFLLLLFIGIQLRNSGLTLRQILLNKHGMIISVVIILSSWAGGIIAAFILDLPIYQSLAMSSGFGWYSLSGILMGDAYGPVFGGASFMLELLRELVALVSIPLMVRNRPCTAIGYAGATAMDFTLPVIQSSGGVRCVPVAIVSGFILSLVVPFFMLFFVSLGPV

SEQ ID NO: 75 - DNA, 921 bp, Neisseriaceae bacterium
atgtttcaattggtaattcttttatttgcccttgttgtgggttactggattaaacgcctgccgctgtccaacaattcgctgaacaaattgctgttcttcatcgtggtcgccattttgtttgtaatgggctatgacttcggcgttaacttaaaagaactgattgaagaattggtcaacatcgtcctgatggtggtagtctttacctcattgattttcttttgtaattttgtttgcgctagcattgtgtttcgtaaggagaataagcaatgccgcgataacctgaaaaacgggccccaagtctcaacgaattatctgctgtacattttgcagtcagcgaagtacaccggaatcattttcttgggcatcatttgtggtgtaatcctccagaaaccgctgacatacttgggggaaattatcgatgttcttctgattgtacttctgttcgtaattggccaccaaatgcgtattagtggcacaaaactgaaacacgtgtttctcaataaaagtgggatcaagttatcactgtctatcgtgtttagttctctgttagcaggcgtgctggtttccctgatcctgaaattccccctgaaccaggctttagctatgtcgagcggctttggttggtacacgttatcgagtatcatggtcggtagtctgatcaatgaaaactactccgtggtttcattcttcattgattttaaccgtgaactgattgccattatcctgctgccgtcgttaggtcgcctgtttccgtatactatggtcggtgtgtgcggcgcgaccgcgatggacttcagccttccggtgattaaacaaaacctttcgcagcagctggtggtggttgccatctccagcggaatgatcctgagtgtggcaacgccagttctgcttccgctgtttgccaaattgagctacttatag

SEQ ID NO: 76 - protein, 306 aa, Neisseriaceae bacterium
MFQLVILLFALVVGYWIKRLPLSNNSLNKLLFFIVVAILFVMGYDFGVNLKELIEELVNIVLMVVVFTSLIFFCNFVCASIVFRKENKQCRDNLKNGPQVSTNYLLYILQSAKYTGIIFLGIICGVILQKPLTYLGEIIDVLLIVLLFVIGHQMRISGTKLKHVFLNKSGIKLSLSIVFSSLLAGVLVSLILKFPLNQALAMSSGFGWYTLSSIMVGSLINENYSVVSFFIDFNRELIAIILLPSLGRLFPYTMVGVCGATAMDFSLPVIKQNLSQQLVVVAISSGMILSVATPVLLPLFAKLSYL

SEQ ID NO: 77 - DNA, 903 bp, Proteus myofaciens ATCC 19692
atgctgtctggcctgctgatcattctgttaccccttttcgtgggctatcttatcaaactgaacaacctgcccttgcttaagatcgccaaccgcctgttaagcgcgatggtgtacgtgatcctgttcctgatgggtgtgtctctggctatgctggataacatcggtgaaaatttagttagcatcctgttttacgcgtcagtatttttcttctgcacttttggtgcgaacctcttgtgcttatggctgctggataagaaagatccgtggcacgtcccggcacacaaacagagtaaaccgccctcccgcatcaaaatggtcctcgaatctctgcagttatgtggtgtggttgtcattggcttcttcgtcggtctgacgcaatggcctatttttcactacgcgtcacatgcttcacaaggcgcgctgattttcctgctgtggcttgtggggttgcagctccgtaattcagggatgtctccaaaacaaatcttgattaatcgccgcgggacgaccgttgcgatcgtgatgggtgtatcggcgcttgcgggcggtgtgttggccgcgtacattttggggctgccgacgaaaatgggtctggctattgcaagtggttacggttggtattcgctgtccggtatcgtgttgaccgatgccttcggaccggtcatcgggtccacggcgttcttcaatgatctcatgcgtgaactggccgcgatcatgctgatccccattatcgtcaaccgttatcgcaacaccgctctgggtatttgtggttctacttcaatggatttcacgctccctgtgctgcagcgctctggtggtgttgcgattattcctgcggcaattgtgcatggttttgttctgtctttaattacaccaattttgatggcattctttacctcatag

SEQ ID NO: 78 - protein, 300 aa, Proteus myofaciens ATCC 19692
MLSGLLIILLPLFVGYLIKLNNLPLLKIANRLLSAMVYVILFLMGVSLAMLDNIGENLVSILFYASVFFFCTFGANLLCLWLLDKKDPWHVPAHKQSKPPSRIKMVLESLQLCGVVVIGFFVGLTQWPIFHYASHASQGALIFLLWLVGLQLRNSGMSPKQILINRRGTTVAIVMGVSALAGGVLAAYILGLPTKMGLAIASGYGWYSLSGIVLTDAFGPVIGSTAFFNDLMRELAAIMLIPIIVNRYRNTALGICGSTSMDFTLPVLQRSGGVAIIPAAIVHGFVLSLITPILMAFFTS

SEQ ID NO: 79 - DNA, 891 bp, uncultured Bacteroides sp.
atgaagaaactgagcaaaaatgcactgatcggatatccggctggtatcatcacgggcattacctatggcttaaatccactttttgccgtaccgctgatgaaacatggggccgctaccgaaagcatcctgttctttcgttacctgtttgcggtattaattttagccctgttcctgcttgttcgccgccagagctttcgcgtgagcggccaacaggcaggaatcctgctcattctgggactcctgtacacctcgagttcccttttcttatttgatgcgtatgaatatattgcaagtgggcttgctaccaccttggtttttctgtacccggtcctggtcgcaattatcatggtcttcttgaaagttgttccgagctggccagtgtggctggcaattgctgctacctttgggggagtgctcattatgacccaaggtgatggcacccaggcgttaaatccgatcggtgttttgctgtcaatcggtagcgccctggtgtacgccttgttcattgtcattattaaccgttcgaaggcgattgcgtccatttcaaactcactgctgactttttatgcgttgtcggtaggagccgttgttttcctgggcaagattctgatctcggacaccgcactgaccgcaggcctgaatcatgctcacgattggctgaatctggtgggccttgcattccttccgactgttgtctcgacagccacccttgcgattgcgagccgcaacattggcgctaccaaggcgagcgtcctcggcgtattcgagcctattacggcgatcgtcgtcggtaccctggtgttcggtgagccgttgaccacaaacattgtggtcggtatcaccgtagccattgtggccgtgacgtttatgatcaccttgacgaaacgttag

SEQ ID NO: 80 - protein, 296 aa, uncultured Bacteroides sp.
MKKLSKNALIGYPAGIITGITYGLNPLFAVPLMKHGAATESILFFRYLFAVLILALFLLVRRQSFRVSGQQAGILLILGLLYTSSSLFLFDAYEYIASGLATTLVFLYPVLVAIIMVFLKVVPSWPVWLAIAATFGGVLIMTQGDGTQALNPIGVLLSIGSALVYALFIVIINRSKAIASISNSLLTFYALSVGAVVFLGKILISDTALTAGLNHAHDWLNLVGLAFLPTVVSTATLAIASRNIGATKASVLGVFEPITAIVVGTLVFGEPLTTNIVVGITVAIVAVTFMITLTKR

SEQ ID NO: 81 - DNA, 891 bp, Phocaeicola plebeius
atgaaaaagttaagtaaaaatgcaattattggatatcctgctggtattattaccggcattacttatggcctgaatccactgttcgcaatgccattaatgaaaaatggtgctgcgaccgaatctatcctgtttttccgctatgcgtttgccgtgctgttactgggcctgtttctgttgtttcgtaagcagtcattccgcgtatcggggaaacagattggcgtactctttattctgggcctgctttatacttcgtcttcgatctttttgtttgacgcgtatgaatatatcgcctcgggtctggccaccactctggtgtttctgtacccggtgcttgtggcgattatcatggtttttctgaaagtagttcccagctggccggtgtggttagctatcgccgcgactttcggcggcgtgttaatcatgacccagtcagatggctcgcagactattaatcctattggcgtgttgctgagcatcgcttcagctttggtatatgcattatttattgtcatcatcaaccgttctaaagcgattgcgggaattagcaattctctgctgaccttttacgccttgatggtaggcgccatcgtgttcattggtaaaatcctcagcagtgataccgcgattacggcaggcatcaccacaggggcagattggttaaatcttgtaggtctggctctgctgccaacgatcgtttctactgctaccctggcgatcgcgtcccgtaatatcggagcgaccaaagcaagtgttctgggcgtttttgaaccgatcaccgctatcattgttggcaccctgatgttcggtgagcctctgaccaccaacattatcgttggtatctcgattgcgatggttgcggttacttttatgatcactgtgacgaaacgttag

SEQ ID NO: 82 - protein, 296 aa, Phocaeicola plebeius
MKKLSKNAIIGYPAGIITGITYGLNPLFAMPLMKNGAATESILFFRYAFAVLLLGLFLLFRKQSFRVSGKQIGVLFILGLLYTSSSIFLFDAYEYIASGLATTLVFLYPVLVAIIMVFLKVVPSWPVWLAIAATFGGVLIMTQSDGSQTINPIGVLLSIASALVYALFIVIINRSKAIAGISNSLLTFYALMVGAIVFIGKILSSDTAITAGITTGADWLNLVGLALLPTIVSTATLAIASRNIGATKASVLGVFEPITAIIVGTLMFGEPLTTNIIVGISIAMVAVTFMITVTKR

SEQ ID NO: 83 - DNA, 909 bp, Xenorhabdus bovienii
atgtactctggtttattaatcgttctgattccgttgattctgggttatcttatccgtttaaataataaaacagcgctggccacggtgcactacttattaaatatcatgatctatgtgatcctgtttctgatgggtgtccgcttagcgatgctcgagaacttgggaaacaatctcctgagtatcctgctgtatgctatgactttctttctttgcatctttgcaacgaatcttctcgcgctcattctcttggataaacgcgacccgtggatcattcaggttaacaaacaggaaaagtccccatcgcagctgcatattgtgtcagatagtattaaactttgcggagcgttaattcttggttttctgttgggtctgacggactggagcctgttccattttgcttccccggctagtgaaattactttgattctgcttcttctccttgtgggcattcagttgcgtaacaacggcatgtctttaaaacagactctcctgaatcgtcgtggtaccattatcgcgcttgttgtcgcgatttcttcgctgcttggcggtatgatcgcagccttcttactgggattgccaacgaaaacgggtttagccatcgcgagtggttatgggtggtattcgctgagcgggatcctcctgtcggacgcatacgggaccgtaatcggcagcgcagctttctttaatgacctggctcgtgaattggcgagtatcctgctcattcctatgcttattaaccgttaccgctcaaccgccctgggcttgacgggagcggcatctattgacttcaccctgccgattctgcagcgctgcggtggaattagcattgtgcctgccgcgatcgttcatggctttatcttatcactgatgacgccagtgtttattgcgtttttcacccagcaagcgtag

SEQ ID NO: 84 - protein, 302 aa, Xenorhabdus bovienii
MYSGLLIVLIPLILGYLIRLNNKTALATVHYLLNIMIYVILFLMGVRLAMLENLGNNLLSILLYAMTFFLCIFATNLLALILLDKRDPWIIQVNKQEKSPSQLHIVSDSIKLCGALILGFLLGLTDWSLFHFASPASEITLILLLLLVGIQLRNNGMSLKQTLLNRRGTIIALVVAISSLLGGMIAAFLLGLPTKTGLAIASGYGWYSLSGILLSDAYGTVIGSAAFFNDLARELASILLIPMLINRYRSTALGLTGAASIDFTLPILQRCGGISIVPAAIVHGFILSLMTPVFIAFFTQQA

SEQ ID NO: 85 - DNA, 903 bp, Pragia fontium
atgtacagcggcctggccattattctgttacctctgatctttggctatctgattcctatcaagagtttggatctgttgcgtacaattaatcgtctgttgtcaagtatggtttatgtgatcctgttctttatgggaattagtctggcattcctggacaacctgtctgcaaatttacagatgatctttttgtatgcctccgttttctttatttgcatcttctctagtaatatcctggcactgatgcttttggatcgccagtggccgtggcgtaaccgtcagcatcagaagaaacttcccagtcgcctgcacatggtgcttgaatcacttcgcctgtgcggtgtggtggtgatcggcttcctgcttggtttaacccacatttcgtggttggcatacgcaagccaggccagccagttcgcgttaatcttcctcctggtgctggtaggtctgcagttgcgtaactcgggcatgaccttacgtgaaattacactgaacaaacgcggtatggttgtcgcacttattgtcgtcattagcgcatttgccggcggcgctattggtgcgcttttgctgggcctgccagtgaaaactggcctggcgatgagctcggcttatggctggtacagcctttcggggattctcctgactgatgccctgggtccagtgctgggtagtgcagcatttttcaacgatctggctcgtgaattggtggcgattatgctgattccgactctgattcgcagccatgcgagtaccgcgttaggcatttgtggtgcaacttccatggatttcaccctgccagtcttacagcgctcaggcagcgttgaaatcatcccggctgcaattgtgcacgggtttatcttgtctctgctggcaccaattctgatggcgttcttttcggcgccgtag

SEQ ID NO: 86 - protein, 300 aa, Pragia fontium
MYSGLAIILLPLIFGYLIPIKSLDLLRTINRLLSSMVYVILFFMGISLAFLDNLSANLQMIFLYASVFFICIFSSNILALMLLDRQWPWRNRQHQKKLPSRLHMVLESLRLCGVVVIGFLLGLTHISWLAYASQASQFALIFLLVLVGLQLRNSGMTLREITLNKRGMVVALIVVISAFAGGAIGALLLGLPVKTGLAMSSAYGWYSLSGILLTDALGPVLGSAAFFNDLARELVAIMLIPTLIRSHASTALGICGATSMDFTLPVLQRSGSVEIIPAAIVHGFILSLLAPILMAFFSAP

SEQ ID NO: 87 - DNA, 900 bp, Klebsiella quasipneumoniae
atgttcagcggtctgtttatcattctgttaccattggttgtcggctacctgttgccattgcgccatagttcggcgctgaaattgattaatcgtatgttaagttggattgtctatgtgatcctgttcttcatgggcatctcccttgcctttctggataacctggcgagcaatcttctggctatcctgcattatgccgcagtgtcagtagtaattatcctgctgtgcaatattaccgctttgttgtggttagaacgcaagatgccgtggcgtagcctgcatcgccaagaaaaattgccaagccgtttggcgatggcgctggaatctctgcagctttgcggtgtcgtagtccttggattcttgctcggcttgacgggtcttagctttctgcaacacgcgacggaggcgtcggagtataccttgattttcctgctgttcctcgtggggatccagcttcgtaacaacggtatgtctctgcgccagatcgttcttaatcgtcgcgggatgatcgtagcagtcgtggtaactgctagcagcctgctgggtggcattttaaacgcgttcatcttagatctgccgttaaaaaccggtctggccatggcatcgggctttggttggtattcgctcagtggaatccttttgacagaaagctttggtccggtgattggcagtgccgcgttctttaacgacctgtgccgtgaactgctggctattatgctgattccagggttaattcgccgctctcgtagtacggccttaggtttatgtggcgctacgagcatggattttacactgccagtcttgcagcgtagcgggggtgtcgagattgtgcccgccgccattgttcatggctttctgttgagcctcctggtgccgatcctcatcgccttcttcgccgcctag

SEQ ID NO: 88 - protein, 299 aa, Klebsiella quasipneumoniae
MFSGLFIILLPLVVGYLLPLRHSSALKLINRMLSWIVYVILFFMGISLAFLDNLASNLLAILHYAAVSVVIILLCNITALLWLERKMPWRSLHRQEKLPSRLAMALESLQLCGVVVLGFLLGLTGLSFLQHATEASEYTLIFLLFLVGIQLRNNGMSLRQIVLNRRGMIVAVVVTASSLLGGILNAFILDLPLKTGLAMASGFGWYSLSGILLTESFGPVIGSAAFFNDLCRELLAIMLIPGLIRRSRSTALGLCGATSMDFTLPVLQRSGGVEIVPAAIVHGFLLSLLVPILIAFFAA

SEQ ID NO: 89 - DNA, 900 bp, Bosea sp. WAO
atgaccagtgcttgttcaaccaccccagcacgccctgcctccgcaattgacccaattcttttatttacggttccgtttaccgtagttgcatgggcgagtagctttcctgcaatccgtgcagggctgaccggtttcggtccacttgagatggcagcgctgcgctttgcactggctggggtccccgctgccctgtttctcgtgctgacgcgcgccgcgctccccgcgggcaccgacatttggcgttttctcaccggtggcatcgtcttcattgccctgtatgccatcctgctgaatttaggtcaacgcgtggttccagcaggcgcggcgagttttattatcaacacaaatccaattatgaccgcggcactggcaatgctgatcttgaacgagcgtttttcgctggtggcctggcttgggactactctgtctttcgccggtattggtgttatcgcgctgggcaatggcctggatctgaatatcggcatcagtgtgctgctgattctcggcgcggccttttgcaatgcaatttccaccgtggtccaaaaaccattattcgctcgccacaaaccgctgagcgtcgcggcttggaacatggcaattggcggactggcgttgagcccctttctgccgtcagcgctggaacaaattccaacggctccgagccaggctctgtggtcagttgtctacctggcggtggtgccgagtctgattgcatacggcacgtgggcgatcactctgagccgtctgccagcggcacgtgcgtcaaattttcaatacggtgtgccgccgactgccatgctgatttcctttatttggttaggagaaatcccgaccgcgctcgggatcctgggtggtgccatggctctggcgggtgtggtgattgtgaatctcaaacgttag

SEQ ID NO: 90 - protein, 299 aa, Bosea sp. WAO
MTSACSTTPARPASAIDPILLFTVPFTVVAWASSFPAIRAGLTGFGPLEMAALRFALAGVPAALFLVLTRAALPAGTDIWRFLTGGIVFIALYAILLNLGQRVVPAGAASFIINTNPIMTAALAMLILNERFSLVAWLGTTLSFAGIGVIALGNGLDLNIGISVLLILGAAFCNAISTVVQKPLFARHKPLSVAAWNMAIGGLALSPFLPSALEQIPTAPSQALWSVVYLAVVPSLIAYGTWAITLSRLPAARASNFQYGVPPTAMLISFIWLGEIPTALGILGGAMALAGVVIVNLKR

SEQ ID NO: 91 - DNA, 873 bp, Gammaproteobacteria bacterium
atgcatcctggccgcggtctgttaaaacgtcactacgtgtttgctatcacggcgccggtcttttggtcactggccggtgtggtcgtcaagagtctggaacaggccacggaatggcagattaatttttaccgttgcttgagtctgactctgtttgtggcggcagttaccctgattcgctaccgccgttccacgctggctgtcctgcgcatgggcggtctgaaaaccatcatttctggcgcgctgcttagcggcgcaatgttatgtaatgtggtggccctgaaatacaccacagtggcagtagcagtctttgtaatggcggcagccccgattctcgcagctctgttgggtcgtctgttcttaggcgaagaggtgcgtgcgcgcgtgtgggtttccattgtgttggccattctggggattgggattatggttggaggtcgcctgcaaatcggtgataccctcggcgttgcagtagccatcctgggcatcgtgttctttggtatctacgctgtctctcttcgtgtaggcaaaaatgtggacatgaccccggcggtatttttcggcggcgccatcggcacggcagtctcatttagcgtttctgtgggtactggtgtgggcctggtgattccgtggatcgaagcaatgtggtgcaccctgctgggtgtagtgcagttggggttaggctcggtcctgttcgctttggcagcccagggcgtgccagcggtacagttaaccctgtttgcgctgggtgagccactgctggcgccgttatgggtatggcttatgcttgacgatttgccaaccatgaccaccttgatcggcggcgccgtgctgtttgcggcgctggccctgcaagtctctgcaagccgcaaatag

SEQ ID NO: 92 - protein, 290 aa, Gammaproteobacteria bacterium
MHPGRGLLKRHYVFAITAPVFWSLAGVVVKSLEQATEWQINFYRCLSLTLFVAAVTLIRYRRSTLAVLRMGGLKTIISGALLSGAMLCNVVALKYTTVAVAVFVMAAAPILAALLGRLFLGEEVRARVWVSIVLAILGIGIMVGGRLQIGDTLGVAVAILGIVFFGIYAVSLRVGKNVDMTPAVFFGGAIGTAVSFSVSVGTGVGLVIPWIEAMWCTLLGVVQLGLGSVLFALAAQGVPAVQLTLFALGEPLLAPLWVWLMLDDLPTMTTLIGGAVLFAALALQVSASRK

SEQ ID NO: 93 - DNA, 900 bp, Photohabdus khanii NC19
atgtattcaggtctgttaatcattttacttccgcttaccctgggctatttaattcgtcttaataatcagactggcttgaacgcagtacatcgcctgctcaacgcgatggtttacgtgattttattcctgatgggtatctcactggcgctgcttgaagacctcagccgcaacttattctcaatctttcagtatgccaccgtgttcttcttatgcatttttgcagcgaacatgctggcactgtttttgcttgaaaagcgtgacccgtggatcacgaatagccataaacaagaaaaaccgccctcccgccttcacatgattttcgaatctctgaaattatgtggcgttcttattttgggcttcttgctggggctgaccggctggtcatggcttcattttgcctcactggccagcgagatcgcattagttgtgctcttgttactcgtcggcatccagctgcgcaatggcggcatgaaccttaaacaaattctgctgaaccgtcgcggtacaactattgccctcgttgtagccgtgtcagccttggcaggcggggtagtggccgcgctgattctcgggcttccggttaagactggactggctctggcttcgggctacggttggtatagtctgtctggtattttgctgtccgacgcgtatggacctgtcatcggaagtgccgcgttcttcaatgacctcgcgcgtgagctggcggctattatgctcatccctgtcctggtaaaccgttaccgcagctcagccatcggcctgagcggcgctacgtccatggattttaccctgccaatcctgcaacgttgtggcggtgtatcaatcgtgcctgcggcgatcgtgcatggtttcttgctgagcctgttggctccgctgttaattgccttcttcacgtag

SEQ ID NO: 94 - protein, 299 aa, Photohabdus khanii NC19
MYSGLLIILLPLTLGYLIRLNNQTGLNAVHRLLNAMVYVILFLMGISLALLEDLSRNLFSIFQYATVFFLCIFAANMLALFLLEKRDPWITNSHKQEKPPSRLHMIFESLKLCGVLILGFLLGLTGWSWLHFASLASEIALVVLLLLVGIQLRNGGMNLKQILLNRRGTTIALVVAVSALAGGVVAALILGLPVKTGLALASGYGWYSLSGILLSDAYGPVIGSAAFFNDLARELAAIMLIPVLVNRYRSSAIGLSGATSMDFTLPILQRCGGVSIVPAAIVHGFLLSLLAPLLIAFFT